(19) United States
(12) Patent Application Publication
(10) Pub. No.: US 2023/0024461 A1
(43) Pub. Date: Jan. 26, 2023

(54) METHODS FOR DUAL DNA/PROTEIN TAGGING OF OPEN CHROMATIN

(71) Applicant: Beth Israel Deaconess Medical Center, Inc., Boston, MA (US)

(72) Inventors: Jonathan LEE, Boston, MA (US); Sean CLOHESSY, Boston, MA (US); Pier Paolo PANDOLFI, Boston, MA (US)

(21) Appl. No.: 17/781,989

(22) PCT Filed: Dec. 2, 2020
(86) PCT No.: PCT/US2020/062878

§ 371 Date: Jun. 2, 2022

Related U.S. Application Data
(60) Provisional application No. 62/942,522, filed on Dec. 2, 2019.

(51) Int. Cl. G01N 33/68; C12N 9/22; C12N 9/08; C12Q 1/28

(52) U.S. Cl. CPC G01N 33/6875; C12N 9/22; C12N 9/0065; C12Q 1/28

(57) ABSTRACT

The invention provides methods, compositions, and kits for characterizing open chromatin by dual DNA/protein tagging.

### SEQUENCE LISTING

The instant application contains a Sequence Listing which has been submitted electronically in ASCII format and is hereby incorporated by reference in its entirety. Said ASCII copy, created on Dec. 20, 2020, is named “01948-268WO2_Sequence_Listing_12_2_20_ST25” and is 73,214 bytes in size.

### FIELD OF THE INVENTION

This invention is in the field of epigenomic analysis.

### BACKGROUND

In the eukaryotic cell, DNA and protein intertwine as chromatin, forming a dynamic epigenomic landscape comprising of genes, their regulatory sequence elements, and the transcription factor complexes modulating their expression at these regulatory sequences (Kornberg et al., Annu. Rev. Cell Dev. Biol. 8:563-587, 1992; Gerstein et al., Nature 489:91-100, 2012; Lambert et al., Cell 172:650-665, 2018). A prerequisite for the function of the regulatory elements is the ability of transcription factor components to access the encoded DNA elements, otherwise impinged by nucleosomal occupancy or higher-order steric hindrance (Dann et al., Nature 548:607-611, 2017; Allis et al., Nat. Rev. Genet. 17:487-500, 2016). Regions of open chromatin constitute approximately 2-3% of the genome and are continuously remodeled to control access of transcriptional machinery and to modulate gene expression (Klemm et al., Nat. Rev. Genet. 20:207-220, 2019; Thurman et al., Nature 489:75-82, 2012). Thus, a comprehensive profile of accessible genomic regions and their associated proteomes would provide a framework to understand genome-wide transcriptional regulation, especially as it applies to cellular identity or disease.

While sequence-based profiling methods of open chromatin, such as DNase hypersensitivity (Thurman et al., Nature 489:75-82, 2012; Boyle et al., Cell 132:311-322, 2008) and the assay for transposase-accessible chromatin using sequencing (ATAC-seq) (Buenrostro et al., Nat. Methods 10:1213-1218, 2013), have expanded our understanding of the regulation of chromatin states and transcription, global profiling of transcription factor substrates associated with accessible chromatin regions still remains inferential from these data sets (Sung et al., Nat. Methods 13:222-228, 2016). Specifically, successful identification of transcription factor binding via bioinformatic “footprinting” approaches is mostly limited to those sequence-specific transcription factors with long residence times on chromatin, despite known binding and activity of a number of transcription factors with undetectable footprints (Sung et al., Nat. Methods 13:222-228, 2016; Baek et al., Cell Rep. 19:1710-1722, 2017). On the other hand, mass spectrometry-based methods have emerged to characterize the protein components associated with open chromatin directly such as through differential chromatin fragmentation (Wierer et al., Hum. Mol. Genet. 25:R106-R114, 2016; Torrente et al., PLoS One 6:e24747, 2011; Alajem et al., Cell Rep. 10:2019-2031, 2015; Dutta et al., Mol. Cell. Proteomics 13:2183-2197, 2014; Kulej et al., Mol. Cell. Proteomics 16:S92-S107, 2017), and yet these approaches do not readily specify the differentially bound genomic loci.

Methods are needed for comprehensive characterization of genomic, proteomic, and transcriptomic features of open chromatin.

### SUMMARY

The invention provides methods for analyzing open chromatin, the methods including: (a) fragmenting and tagging accessible genomic DNA of the open chromatin, and (b) labeling molecules proximal to the accessible genomic DNA.

In some embodiments, the fragmenting, tagging, and labeling is carried out by treating the open chromatin with a fusion protein including (a) a first enzyme that fragments and tags the accessible genomic DNA of the open chromatin, and (b) a second enzyme that labels molecules proximal to the accessible genomic DNA.

In some embodiments, the molecules proximal to the accessible genomic DNA are proteins, peptides, or RNA molecules.

In some embodiments, the methods further include the step of characterizing one or both of (a) genomic DNA fragments tagged by the first enzyme, and (b) proteins or peptides labeled with the second enzyme.

In some embodiments, the first enzyme is selected from the group consisting of a transposase, a retroviral integrase, a DNA-binding enzyme, or a variant thereof.

In some embodiments, the transposase is selected from the group consisting of a Tn transposase, a hAT transposase, a DD[E/D] transposase, and variants thereof.

In some embodiments, the Tn transposase is selected from the group consisting of Tn3, Tn5, Tn7, Tn10, Tn552, Tn903, Tn/O, TnA, and variants thereof.

In some embodiments, the Tn transposase is Tn5 or a variant thereof, such as Tn5-059.

In some embodiments, the DNA-binding enzyme is selected from the group consisting of a DNase, an MNase, a restriction enzyme, and variants thereof.

In some embodiments, the second enzyme is selected from the group consisting of a peroxidase, a biotin ligase, a catalase-peroxidase, and an oxidase.

In some embodiments, the peroxidase is selected from the group consisting of ascorbate peroxidase (APX), horseradish peroxidase (HRP), soybean ascorbate peroxidase, pea ascorbate peroxidase, Arabidopsis ascorbate peroxidase, maize ascorbate peroxidase, cytochrome c peroxidase, laccase, tyrosinase, and variants thereof.

In some embodiments, the second enzyme includes an ascorbate peroxidase selected from APEX2, APEX, and variants thereof.

In some embodiments, the first enzyme includes Tn5, or a variant thereof, and the second enzyme includes APEX2, or a variant thereof.

In some embodiments, the fusion protein includes a linker between the first and second enzymes.

In some embodiments, the fusion protein includes a tag.

In some embodiments, the first enzyme tags genomic DNA fragments generated by the first enzyme with sequencing adaptors, and/or the second enzyme labels molecules proximal to the accessible genomic DNA with biotin.

In some embodiments, the methods include the use of two fusion proteins, wherein the first fusion protein includes the first enzyme fused to a portion of the second enzyme, and the second fusion protein includes the first enzyme fused to a second portion of the second enzyme.

In some embodiments, the first and second fusion proteins are used together or are used sequentially.

In some embodiments, the characterization of the tagged genomic DNA fragments includes sequencing.

In some embodiments, the characterization of the labeled proteins or peptides includes mass spectrometry analysis.

In some embodiments, the methods further include cross-linking of RNA molecules proximal to accessible genomic DNA to proximal peptides and proteins, and analyzing the cross-linked RNA molecules by RNAseq.

In some embodiments, the open chromatin is obtained from cells of a subject or from cultured cells.

In some embodiments, the cells of a subject are included within a tissue biopsy or a blood sample.

In some embodiments, the tissue biopsy is a tumor biopsy.

In some embodiments, the methods further include the step of characterizing (a) genomic DNA fragments tagged by the first enzyme, and (b) proteins or peptides labeled with the second enzyme.

In some embodiments, the methods further include the preparation of an epigenetic map of a region of the genome of a cell based on the characterization of tagged genomic DNA fragments, labeled RNA, labeled proteins, or labeled peptides.

In some embodiments, the methods further include preparing an epigenetic profile associated with a disease or condition, the method including carrying out a method as described above or elsewhere herein on a sample including cells of a subject having the disease or condition, or a model thereof.

The invention further includes methods for determining whether a subject has a disease or condition associated with an epigenetic profile, the methods including carrying out a method as described above or elsewhere herein on a sample from the subject.

The invention additionally provides methods for monitoring the progress of treatment a disease or condition associated with an epigenetic profile, the methods including carrying out a method as described above or elsewhere herein on a sample from the subject (i) before and (ii) during or after treatment of the disease or condition.

Further, the invention provides methods for determining the effects of exposure of a subject to a biological or chemical stimulus, the methods including carrying out a method as described above or elsewhere herein on a sample from the subject after exposure to the biological or chemical stimulus.

The invention additionally provides methods for identifying the components of a cis-regulatory transcription factor network, the methods including carrying out a method as described above or elsewhere herein on a sample including cells of interest.

The invention further provides methods for identifying a target for drug development against a disease, the methods including carrying out a method as described above or elsewhere herein on a sample including cells characteristic of the disease and identifying one or more molecules, the presence or abundance of which is changed in the cells characteristic of the disease, relative to a control.

The invention also further provides fusion proteins including (a) a first enzyme that fragments and tags accessible genomic DNA of open chromatin, and (b) a second enzyme that labels molecules proximal to the accessible genomic DNA, or a portion thereof.

In some embodiments, the first enzyme includes a transposase, a retroviral integrase, a DNA-binding enzyme, or a variant thereof.

In some embodiments, the transposase is selected from the group consisting of Tn transposases, hAT transposases, DD[E/D] transposases, and variants thereof.

In some embodiments, the Tn transposase is selected from the group consisting of Tn3, Tn5, Tn7, Tn10, Tn552, Tn903, Tn/O, and TnA, and variants thereof.

In some embodiments, the Tn transposase is Tn5 or a variant thereof, such as Tn5-059.

In some embodiments, the DNA-binding enzyme is selected from DNase, MNase, restriction enzymes, and variants thereof.

In some embodiments, the Tn transposase includes the sequence of SEQ ID NO: 2, or a variant thereof.

In some embodiments, the second enzyme is selected from the group consisting of a peroxidase, a biotin ligase, a catalase-peroxidase, and an oxidase, or a portion thereof.

In some embodiments, the peroxidase is selected from the group consisting of ascorbate peroxidase (APX), horseradish peroxidase (HRP), soybean ascorbate peroxidase, pea ascorbate peroxidase, Arabidopsis ascorbate peroxidase, maize ascorbate peroxidase, cytochrome c peroxidase, laccase, tyrosinase, and variants thereof.

In some embodiments, the second enzyme includes an ascorbate peroxidase selected from APEX2, APEX, and variants thereof.

In some embodiments, the APEX2 includes the sequence of SEQ ID NO 4, or a variant thereof.

In some embodiments, the first enzyme includes Tn5, or a variant thereof, and the second enzyme includes APEX2, or a variant thereof.

In some embodiments, the first enzyme is N-terminal to the second enzyme.

In some embodiments, the second enzyme is N-terminal to the first enzyme.

In some embodiments, the fusion protein includes a linker between the first enzyme and the second enzyme.

In some embodiments, the linker includes a sequence selected from SEQ ID NOs: 7, 9, 11, and 13.

In some embodiments, the fusion protein further includes a tag.

In some embodiments, the tag includes a Flag tag.

In some embodiments, the Flag tag includes the sequence of SEQ ID NO: 15 or 16.

The invention also provides nucleic acid molecules encoding a fusion protein as described above or elsewhere herein.

In some embodiments, the nucleic acid molecule includes the sequence of SEQ ID NO: 1 or SEQ ID NO: 3.

The invention additionally provides cells including a nucleic acid molecule as described above or elsewhere herein or expression a fusion protein described above or elsewhere herein.

The invention further provides vectors including a nucleic acid molecule described above or elsewhere herein.

Also, the invention provides kits including (a) a fusion protein, a nucleic acid molecule, a cell, or a vector as described above or elsewhere herein, and/or (b) one or more reagents for carrying out a method described above or elsewhere herein.

Furthermore, the invention includes kits including (i) (a) a first fusion protein including a first enzyme that fragments and tags accessible genomic DNA of open chromatin, and (b) a first portion of a second enzyme, and (ii) a second fusion protein including the first enzyme and a second portion of the second enzyme, wherein the first and second portions of the second enzyme together label molecules proximal to the accessible genomic DNA.

The invention also provides methods for characterizing changes in open chromatin, the methods including carrying out a method described herein, involving fragmenting, tagging, and labeling, as described herein, with chromatin from or present in cells subject to different conditions or at different times, and classifying transcription factors identified as being associated with the open chromatin with respect to abundance or activity under the different conditions or at the different times.

In some embodiments, the abundance of identified transcription factors is characterized as being decreased, unchanged, or increased.

In some embodiments, the activity of identified transcription factors is characterized as being closed, unchanged, or open.

In some embodiments, both abundance and activity of identified transcription factors is classified.

In some embodiments, the different conditions are selected from exposure to drug treatment or a physiological change.

In some embodiments, the different times are different stages of development or different times before, during, or after therapeutic intervention.

In some embodiments, the methods further include determining relationships between transcription factors, determining their functions, identifying them as therapeutic targets, identifying them as transcriptional activators, or identifying them as transcriptional repressors.

In some embodiments, the methods further include identification of transcription factor networks as related to one another and cis-acting sequences.

In some embodiments, the methods further include identification of protein complex dynamics.

Unless defined otherwise herein, all technical and scientific terms used herein have the same meaning as commonly understood by those of ordinary skill in the art to which this invention belongs. All patents and publications referred to herein are expressly incorporated herein by reference. Numeric ranges are inclusive of the numbers defining the ranges. Unless otherwise indicated, nucleic acid molecules are written left to right in 5′ to 3′ orientation and amino acid sequences are written left to right in amino to carboxyl orientation. The term “a” includes one or more unless context indicates otherwise.

The term “sample” as used herein refers to material or a mixture of materials that may contain one or more analytes of interest (e.g., open chromatin). In some examples, the term refers to any animal (e.g., human), plant, or microbial material or mixtures thereof containing any one or more of the following types of molecules: DNA, RNA, proteins, peptides, carbohydrates, lipids, fats, and/or other organic molecules. Such samples include, for example, tissue, cells, or fluid isolated from a subject (e.g., a mammal, such as a human). Specific examples of materials or mixtures thereof which form the basis of a “sample” include blood (e.g., whole blood and peripheral blood samples), biopsy material (e.g., tumor or tissue samples), cerebrospinal fluid, and tissue sections. Samples can be obtained from a “subject,” e.g., a mammal such as a patient (e.g., a human patient).

The terms “determining,” “measuring,” “assessing,” “assaying,” and “analyzing” can be used interchangeably herein to refer to any form of measurement. These terms include quantitative and/or qualitative determinations, and further include determining whether an element is present or not. The determinations can be relative to a control or absolute.

The term “chromatin,” as used herein, refers to a complex including molecules such as proteins and polynucleotides (e.g., DNA and/or RNA) and can be found, e.g., in the nucleus of a eukaryotic cell or isolated therefrom. Chromatin can include histone proteins that form nucleosomes, genomic DNA, RNA, and DNA binding proteins (e.g., transcription factors) that are generally associated with (e.g., bound to) the genomic DNA. “Chromatin” also refers to complexes of DNA, protein, and/or RNA that are extracted from eukaryotic cells. “Open chromatin” refers to a region of chromatin in which DNA is accessible by, e.g., proteins (e.g., transcription factors and/or the fusion proteins as described herein).

The term “region,” as used herein, can refer to a contiguous length of nucleotides in the genome of a cell or organism. A chromosomal region can be in the range of, e.g., 1 base pair to the length of an entire chromosome. In some examples, a region can have a length of at least 200 bp, at least 500 bp, at least 1 kb, at least 10 kb or at least 100 kb or more (e.g., up to 1 Mb or 10 Mb or more). The genome can be from any eukaryotic organism, e.g., an animal or plant genome, such as the genome of a human or other animal.

The term “proximal” as used herein is not to be limited by any particular distance. Rather, the term is used to refer to molecules that are close enough to open chromatin as described herein, such that they are labeled when the open chromatin is fragmented and tagged using a fusion protein as described herein.

The term “epigenetic map,” as used herein, refers to any representation of epigenetic features, e.g., sites of nucleosomes, nucleosome-free regions, binding sites for transcription factors, etc.

The terms “polypeptide” and “peptide” and “protein” are used interchangeably herein and refer to polymers of amino acids of any length. The polymer can be linear or branched, it can include one or more modified amino acids or analogs, and/or it can be interrupted by non-amino acids. The terms also include amino acid polymers that have been modified naturally or by intervention, e.g., by disulfide bond formation, glycosylation, lipidation, acetylation, phosphorylation, and/or any other manipulation or modification, such as labeling.

A “conservative amino acid substitution” is one in which one amino acid residue is replaced with another amino acid residue having a similar side chain with respect to, e.g., length, charge, and other molecular features. Families of amino acid residues having similar side chains are generally defined in the art to include those with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine), and aromatic side chains (e.g., tyrosine, phenylalanine, tryptophan, and histidine). Generally, conservative substitutions in the sequences of the polypeptides (e.g., the fusion proteins) of the invention do not disrupt the activities thereof.

The term “fusion protein” or “fusion polypeptide” as used herein refers to a protein or polypeptide including sequences from two or more proteins or peptides that do not naturally occur together within the same molecule (e.g., they are not naturally produced together). Fusion proteins can be encoded by a nucleic acid molecule including two or more coding sequences. Optionally, the components of a fusion protein are fused directly to one another. In other examples, the components of a fusion protein are connected to one another by a linker sequence. The term “linker” as used herein refers to a linker inserted between a first polypeptide and a second polypeptide (e.g., a first and second polypeptide of a fusion protein as described herein). In some examples, the linker is a peptide linker (e.g., a flexible linker including glycine residues).

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. In some examples, a “polynucleotide” or “nucleic acid” is a nucleotide-containing polymer of any length (e.g., at least 2, 10, 100, 500, 1000, 5,000, 10,000, 100,000, 1,000,000 bases or more). The terms includes single- and double-stranded molecules, which can include deoxyribonucleotides, ribonucleotides, modified versions thereof, and/or mixtures thereof. Naturally-occurring nucleotides include guanine, cytosine, adenine, thymine, uracil (G, C, A, T, and U, respectively). DNA and RNA have deoxyribose and ribose sugar backbones, respectively. Modified nucleic acid molecules and nucleic acid analogs, which can include, e.g., modified bases and/or sugar backbones, are included in the invention. The term “oligonucleotide” as used herein typically refers to a single-stranded polynucleotide of, e.g., from about 2 to 300 nucleotides (e.g., 10 to 20, 21 to 30, 31 to 40, 41 to 50, 51 to 60, 61 to 70, 71 to 80, 80 to 100, 100 to 150, 150 to 200, 200 to 250, or 250 to 300), up to 500 to 1000 nucleotides in length. Oligonucleotides can contain ribonucleotide monomers, deoxyribonucleotide monomers, both ribonucleotide monomers and deoxyribonucleotide monomers, and/or modified versions thereof.

The term “barcode label,” as used herein, refers to a sequence of nucleotides that can be used to identify and/or track the source of a polynucleotide in a reaction, and/or count how many times an initial molecule is sequenced. A barcode label can be at the 5′-end, the 3′-end, or in the middle of nucleic acid molecule such as an oligonucleotide, and can have a length of, e.g., from 4 to 40, 6 to 30, or 8 to 20 nucleotides.

The term “vector” as used herein is a construct that is capable of delivering, and usually expressing, one or more gene(s) or sequence(s) of interest in a host cell. “Expression vectors” are vectors including regulatory sequences (e.g., a promoter), and into which heterologous nucleotide sequences to be expressed are inserted in operable linkage with the regulatory sequences. Expression vectors include, e.g., cosmids, plasmids (e.g., naked or contained in liposomes), and viruses (e.g., lentivirus, retroviruses, adenoviruses, and adeno-associated viruses), and modified versions thereof. The term “operably linked” refers to functional linkage between regulatory sequences (e.g., promoters) and heterologous nucleic acid sequences, which results in expression of the latter. As used herein, a “promoter” is nucleic acid sequence that directs transcription of a polynucleotide sequence.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, e.g., BLAST, ALIGN, Megalign, BestFit, GCG Wisconsin Package, and variants thereof. In some embodiments, two nucleic acids or polypeptides of the invention are substantially identical, meaning that they have at least 70%, at least 75%, at least 80%, at least 85%, at least 90%, or in some examples at least 95%, 96%, 97%, 98%, 99% nucleotide or amino acid residue identity, when compared and aligned for maximum correspondence, as measured using a sequence comparison algorithm or by visual inspection. In some examples, identity exists over a region of the amino acid sequences that is at least about 10 residues, at least about 20 residues, at least about 40-60 residues, at least about 60-80 residues in length, or any integral value there between. In some embodiments, identity exists over a longer region than 60-80 residues, such as at least about 80-100 residues, and in some embodiments the sequences are substantially identical over the full length of the sequences being compared. In some embodiments, identity exists over a region of the nucleotide sequences that is at least about 10 bases, at least about 20 bases, at least about 40-60 bases, at least about 60-80 bases in length, or any integral value there between. In some embodiments, identity exists over a longer region than 60-80 bases, such as at least about 80-1000 bases or more, and in some embodiments the sequences are substantially identical over the full length of the sequences being compared.

A polypeptide, polynucleotide, vector, cell, or other composition that is “isolated” is a polypeptide, polynucleotide, vector, cell, or other composition that is in a form not found in nature. Isolated polypeptides, polynucleotides, vectors, cells, or compositions include, e.g., those that have been purified to a degree that they are no longer in a form in which they are found in nature. In some examples, a polypeptide, polynucleotide, vector, cell, or composition that is isolated is substantially pure. The term “substantially pure,” as used herein, refers to material that is at least 50% pure (e.g., free from contaminants), at least 90% pure, at least 95% pure, at least 98% pure, or at least 99% pure.

Other features and advantages of the invention will be apparent from the following detailed description, the drawings, and the claims.

### BRIEF DESCRIPTION OF THE DRAWINGS

FIG. 1|Transposase/peroxidase fusion probes tag DNA at regions of open chromatin. a, Schematic of integrative DNA And Protein Tagging (iDAPT). TP, transposase/peroxidase fusion protein. b, Integrative Genomics Viewer (IGV) genome track view of ATAC-seq (Nextera Tn5, Tn5-F) and iDAPT-seq (TP3, TP5) libraries at a ubiquitously accessible control region. Libraries were generated from the GM12878 cell line. c, Scatterplots comparing genome-wide transposon insertion frequencies of Nextera Tn5 (ATAC-seq) with in-house Tn5-F (ATAC-seq) and of Nextera Tn5 (ATAC-seq) with the transposase/peroxidase fusion TP3 (iDAPT-seq) in the GM12878 cell line. Pearson correlation coefficients are displayed inline. d, Representative images of co-immunofluorescence staining of markers of active transcription (RNA Pol II S2P, H3K27Ac) and repressed transcription (H3K9me3) with ATAC-see using TP3 in the HT1080 cell line. Scale bars, 5 μm. e, Distribution of Pearson correlation coefficients between TP3 ATAC-see and immunostaining of transcription activity markers per nucleus as shown in (d). Numbers of nuclei assessed per marker are displayed inline. Center line, median value; box limits, upper and lower quartiles; whiskers, 1.5× interquartile range; points, outliers.

FIG. 2|Optimization of transposase/peroxidase fusion probes for transposase activity. a, Schematic of recombinant fusion protein linear sequence. PT, peroxidase/transposase; TP, transposase/peroxidase; F, FLAG; L, linker. b, Sequences of protein linkers tested for fusion protein activity. c, Quantitative PCR assessment of pre-amplified GM12878 ATAC-seq libraries generated with the corresponding enzymes (n=1). d, TapeStation DNA HS 5000 assessment of fragment size distributions of GM12878 ATAC-seq libraries. Nucelosomal fragmentation is marked inline. MEDS, Mosaic End double-stranded transposon. e, Gel shift assay of tagmentation reactions of linearized pSMART plasmid with the corresponding enzymes. Gel shift was measured on a 1% agarose gel. f, DNA fragment distributions of (e) assessed on a 1% agarose gel.

FIG. 3|Assessment of transposase activity on native chromatin. a, Ratio of transposon insertions at Ensembl v94 transcription start sites (TSS) relative to background from in-house ATAC-seq/iDAPT-seq and published ATAC-seq libraries (SRR5427884, SRR5427885, SRR5427886, SRR5427887 from Corces et al., Nat. Methods 14:959-962, 2017) generated from the GM12878 cell line (n=1). b, Proportion of non-mitochondrial reads from GM12878 ATAC-seq/iDAPT-seq libraries. c, Heatmap of pairwise Pearson correlation coefficients of genome-wide transposon insertion frequencies for the indicated ATAC-seq/iDAPT-seq libraries. d, Enrichment of ATAC-seq/iDAPT-seq transposon insertions within Ensembl v94 genic features by annotatePeaks.pl from Homer. e, Genome-wide ATAC-seq/iDAPT-seq transposon insertion distributions about CTCF consensus sequences within peaks. f, Fragment size distributions of ATAC-seq/iDAPT-seq libraries. g, Distribution of Pearson correlation coefficients between Tn5-F ATAC-see and immunostaining of transcription activity markers per nucleus. Numbers of nuclei assessed per marker are displayed inline. Center line, median value; box limits, upper and lower quartiles; whiskers, 1.5× interquartile range; points, outliers.

FIG. 4|Assessment of peroxidase activity of transposase/peroxidase (TP) fusion probes. a, Peroxidase activity assessment of purified recombinant enzymes measured by Amplex UltraRed fluorescence in the presence of 1 mM hydrogen peroxide (mean±s.d.; n=5 distinct samples for each condition, single protein purification batch per enzyme). Pairwise two-tailed t-tests with pooled variance were performed, using Holm p-value adjustment to control for family-wise error rate. b, Western blot of relative purified enzyme inputs (FLAG M2). c, Western blot of enzyme retention (FLAG M2) and peroxidase-mediated biotinylation (Streptavidin) in GM12878 nuclei. Ponceau S staining is shown as loading control. d, Quantification of streptavidin-HRP chemiluminescence per lane in (c).

FIG. 5|iDAPT-MS facilitates identification of proteins associated with open chromatin. a, Schematic of iDAPT-MS experimental design and SL-TMT sample labeling for HEK293T profiling. Cells were processed in bulk up to the DNA tagmentation step. b, Volcano plot of proteins enriched by either TP3 or APEX2-F in HEK293T nuclei. Blue points, log 2 fold change >0 and false discovery rate (FDR)<5%; black points, candidate markers of open chromatin (see d); red points, sequence-specific transcription factors. c, ReactomeDB pathways overrepresented in the TP3-labeled nuclear proteome. d, Distribution of eigenvector centrality measures of proteins labeled by TP3 and without non-nuclear subcellular localization annotation. Eigenvector centrality was determined for proteins within the largest connected component of the BioPlex 2.0 network induced by the TP3-labeled nuclear proteome. Red, labeled points, high priority candidate markers of open chromatin. e, Representative images of co-immunofluorescence staining of markers of candidate open chromatin markers CCDCl2 and SNRPA with ATAC-see using TP3 in HT1080 cells. Scale bars, 5 pm. f, Distribution of Pearson correlation coefficients between TP3 ATAC-see and immunostaining of candidate open chromatin markers per nucleus as shown in (e) and in FIG. 7d-f. Numbers of nuclei assessed per marker are displayed inline. Center line, median value; box limits, upper and lower quartiles; whiskers, 1.5× interquartile range; points, outliers.

FIG. 6|iDAPT-MS proteomic enrichment assessment of transposase/peroxidase (TP) fusion probes in HEK293T cells. a, Principal component analysis of proteome profiles from APEX2-F, TP3, and TP5 labeling. b, Volcano plot of proteins enriched by either TP5 or APEX2-F in HEK293T nuclei. Blue points, log 2 fold change >0 and false discovery rate (FDR) <5%; black points, candidate markers of open chromatin; red points, sequence-specific transcription factors. c, Overlap of significant TP3- and TP5-labeled proteomes (limma FDR <5%). d, ReactomeDB pathways overrepresented in the APEX2-F-labeled nuclear proteome. e, Gene Ontology subcellular localization enrichment pattern of the TP3-labeled nuclear proteome. f, Gene Ontology subcellular localization enrichment pattern of the APEX2-F-labeled nuclear proteome. g, Gene Ontology subcellular localization enrichment patterns of published open chromatin proteome profiles.

FIG. 7|Open chromatin marker discovery and validation. a, Prioritization strategy for open chromatin marker curation. b, Largest connected component of the BioPlex 2.0 subgraph induced by enriched TP3 proteins (log 2 fold change >0, FDR <5%) with non-mitochondrial localization annotation. The Fruchterman-Reingold layout algorithm was used for visualization. Red vertices, eigenvector centrality >0.2. c, Coefficient of variance of transcripts per million gene expression levels of candidate open chromatin markers across ˜1,100 cancer cell lines profiled by the Cancer Cell Line Encyclopedia. d-f, Representative images of co-immunofluorescence staining of markers of candidate open chromatin markers CCDCl2 and SNRPA with ATAC-see using TP3 in MDA-MB-231 (d), GM12878 (e), or DU145 (f) cells. Scale bars, 5 pm.

FIG. 8|iDAPT-seq analysis of HEK293T native chromatin versus naked genomic DNA. a, Fragment size distributions of iDAPT-seq libraries generated from the HEK293T cell line or corresponding naked genomic DNA. b, Ratio of transposon insertions at Ensembl v94 transcription start sites (TSS) relative to background from iDAPT-seq libraries (n=1). c, Enrichment of iDAPT-seq transposon insertions within Ensembl v94 genic features by annotatePeaks.pl from Homer. d, Proportion of non-mitochondrial reads from HEK293T iDAPT-seq libraries. e, Principal component analysis of genome-wide transposon insertion frequencies for the indicated iDAPT-seq libraries. f, Volcano plot of iDAPT-seq profiles analyzed with DESeq2. Peak statistics are listed below.

FIG. 9|Integrative analysis of iDAPT-MS and iDAPT-seq enables inference of active sequence-specific transcription factors, their genomic localization patterns, and their protein complex components. a, Schematic of bivariate footprinting analysis of iDAPT-seq data. FPD, footprint depth; FA, flanking accessibility. b, Enrichment of sequence-specific transcription factors from CisBP by iDAPT-seq footprinting analysis and TP3 iDAPT-MS enrichment in HEK293T cells. c, Genome-wide footprint of CTCF in native chromatin (red) and naked DNA (black). The CisBP CTCF motif logo is displayed below. d, Enrichment of ENCODE CTCF ChIP-seq peaks (ENCFF285QVL) among native chromatin iDAPT-seq peaks (DESeq2 log 2 fold change >0, FDR <5%) as compared to naked DNA (DESeq2 log 2 fold change <0). Chi-squared test p-value is reported inline. e, Genome-wide footprint of ZIC2 in native chromatin (red) and naked DNA (black). The CisBP ZIC2 motif logo is displayed below. f, Enrichment of ENCODE ZIC2 ChIP-seq peaks (ENCFF187CEY) among native chromatin iDAPT-seq peaks (DESeq2 log 2 fold change >0, FDR <5%) as compared to naked DNA (DESeq2 log 2 fold change <0). Chi-squared test p-value is reported inline. g, Hierarchical clustering of 79 sequence-specific transcription factors from TP3 iDAPT-MS using motif presence within peaks as binary features. Outer bar chart represents relative number of native chromatin peaks per motif. h, Network view of inferred sequence-specific transcription factor complexes in HEK293T cells, with first order protein interactors from the overlap of BioPlex 2.0 and enriched proteins in TP3 IDAPT-MS. Enriched CORUM complexes are labeled. Red points, sequence-specific transcription factors; black points, associated CORUM complex proteins.

FIG. 10|Comparison of iDAPT-seq and iDAPT-MS enrichment of sequence-specific transcription factors. a, Bivariate footprinting analysis of native chromatin versus naked genomic DNA from HEK293T cells. Red, enriched cluster; blue, non-enriched cluster. b, Two-state Gaussian mixture model using footprint projection along a −45° line for modeling. A probability threshold of 0.5 was used to classify footprints by enrichment. Red, enriched cluster; blue, non-enriched cluster. c, Comparison of enriched sequence-specific transcription factors between iDAPT-seq bivariate footprint analysis and TP3 iDAPT-MS. Overlapping transcription factors are listed below. d, Principal component analysis of ChromVAR enrichment analysis of iDAPT-seq profiles. e, Volcano plot of ChromVAR analysis, using loadings of the first principal component for effect size and FDR-adjusted p-values computed by ChromVAR. FDR threshold <5%. f, Comparison of enriched sequence-specific transcription factors between iDAPT-seq ChromVAR analysis and TP3 iDAPT-MS. Overlapping transcription factors are listed below. g, Genome-wide footprint of YY1 in native chromatin (red) and naked DNA (black). The CisBP YY1 motif logo is displayed below. h, Enrichment of ENCODE YY1 ChIP-seq peaks (ENCFF437JVZ) among native chromatin iDAPT-seq peaks (DESeq2 log 2 fold change >0, FDR <5%) as compared to naked DNA (DESeq2 log 2 fold change <0). Chi-squared test p-value is reported inline. i, Genome-wide footprint of ATF2 in native chromatin (red) and naked DNA (black). The CisBP ATF2 motif logo is displayed below. j, Enrichment of ENCODE ATF2 ChIP-seq peaks (ENCFF225VCG) among native chromatin iDAPT-seq peaks (DESeq2 log 2 fold change >0, FDR <5%) as compared to naked DNA (DESeq2 log 2 fold change <0). Chi-squared test p-value is reported inline. k, Genome-wide footprint of KLF113 in native chromatin (red) and naked DNA (black). The CisBP KLF13 motif logo is displayed below. l, Enrichment of ENCODE KLF13 ChIP-seq peaks (ENCFF880YRF) among native chromatin iDAPT-seq peaks (DESeq2 log 2 fold change >0, FDR <5%) as compared to naked DNA (DESeq2 log 2 fold change <0). Chi-squared test p-value is reported inline.

FIG. 11|iDAPT profiling of mIDH2 AML unravels consequences of R-2HG-mediated epigenomic dysfunction. a, Schematic of iDAPT-MS experimental design and SL-TMT sample labeling for TF1 erythroleukemia cell line profiling. Cell line replicates were taken from the same passage and processed separately. b, Western blot of TF1 cell lines transduced with the indicated pLVX constructs. The IDH2 gene is detected by MYC tag. α-Tubulin is used as loading control. c, LC-MS/MS metabolite profiling of intracellular 2HG levels (mean±s.d.; n=3 repeatedly measured samples for each cell line). Pairwise two-tailed t-tests with pooled variance were performed, using Holm p-value adjustment to control for family-wise error rate. d, Volcano plot of proteins enriched by TP3 in TF1 nuclei transduced with either mutant or wild type IDH2 constructs. Significance is denoted by FDR <5%. Blue points, log 2 fold change >0 and false discovery rate (FDR)<5%; red points, log 2 fold change <0 and FDR <5%; black points, significant proteins of interest. e, ReactomeDB pathway differentially enriched in mutant versus wild type IDH2 cells by gene set enrichment analysis. f, Footprint of GATA1 motifs within differentially closed chromatin peaks (DESeq2 log 2 fold change <0 and p-value <0.05). Insertion rates are smoothed with a 5 bp arithmetic mean window. The CisBP GATA1 motif logo is displayed below. Black, TF1 pLVX-IDH2 (WT); red, TF1 pLVX-IDH2^{R172K} (R172K). g, Gene set enrichment analysis of ENCODE GATA1 ChIP-seq peaks (ENCFF148JKK) from the K562 erythroleukemia cell line. iDAPT-seq peaks are ranked by signed −log 10 p-value by DESeq2. ChIP-seq peaks were downsampled to 2,000 peaks for improved visualization. h, Footprint of TAL1 motifs within differentially closed chromatin peaks (DESeq2 log 2 fold change <0 and p-value <0.05). Insertion rates are smoothed with a 5 bp arithmetic mean window. The CisBP TAL1 motif logo is displayed below. Black, TF1 pLVX-IDH2 (WT); red, TF1 pLVX-IDH2^{R172K} (R172K). i, Gene set enrichment analysis of ENCODE TAL1 ChIP-seq peaks (ENCFF078OUD) from the K562 erythroleukemia cell line. iDAPT-seq peaks are ranked by signed −log 10 p-value by DESeq2. ChIP-seq peaks were downsampled to 2,000 peaks for improved visualization. j, TAL1/GATA1 protein interaction network from BioGrid. Vertex legend is as displayed below. k, Representative flow cytometry plots of TF1 IDH2^{R140Q} knock-in cells (R140Q KI) transduced with either pSIN4 empty vector (EV) or pSIN4-TAL1 open reading frame (TAL1), cultured either with erythropoietin and hemin chloride or normally with GM-CSF (n=1). l, Proposed model of GATA1/TAL1 complex dynamics and disruption due to mIDH1/2. Complex association may either be stepwise as shown or in concert.

FIG. 12|Assessment of TF1 pLVX cell lines for iDAPT-MS proteomic analysis. a, GM-CSF-independent TF1 proliferation assessment (mean±s.d.; n=4 repeatedly measured samples for each cell line). Linear regression of normalized luminescence values was performed using sample type and day as categorical variables with interaction between the two variables (luminescence˜sample+day+sample:day). Reported p-values were from the interaction of sample type with day 13, with WT as baseline. b, Principal component analysis of TF1 LC-MS/MS metabolomic profiles. c, LC-MS/MS metabolite profiling of intracellular 2-oxoglutarate (2OG) and glutamate levels (mean±s.d.; n=3 repeatedly measured samples for each cell line). Pairwise two-tailed t-tests with pooled variance were performed, using Holm p-value adjustment to control for family-wise error rate. d, Principal component analysis of TF1 iDAPT-MS proteomic profiles. e, Gene Ontology subcellular localization enrichment pattern of all detected proteins in TF1 iDAPT-MS. f, Gene set enrichment analysis of annotated R-2HG targets from Losman et al., Genes Dev. 27:836-852, 2013. Detected proteins from iDAPT-MS (mutant versus wild type IDH2 TF1) are ranked by signed −log 10 p-value by limma.

FIG. 13|Assessment of iDAPT-seq from wild type versus mutant IDH2 TF1 cell lines. a, Fragment size distributions of iDAPT-seq libraries generated from the TF1 pLVX cell lines. b, Ratio of transposon insertions at Ensembl v94 transcription start sites (TSS) relative to background from iDAPT-seq libraries (n=1). c, Proportion of non-mitochondrial reads from TF1 iDAPT-seq libraries. d, Enrichment of iDAPT-seq transposon insertions within Ensembl v94 genic features by annotatePeaks.pl from Homer. e, Genome-wide iDAPT-seq transposon insertion distributions about CTCF consensus sequences within peaks. f, Principal component analysis of genome-wide transposon insertion frequencies for the indicated iDAPT-seq libraries. g, Volcano plot of iDAPT-seq profiles using DESeq2. Peak statistics are listed below. h, Bivariate footprinting analysis of mutant versus wild type IDH2 TF1 iDAPT-seq profiles. Red, enriched cluster; blue, non-enriched cluster. i, Two-state Gaussian mixture model using footprint projection along a −45° line for modeling. A probability threshold of 0.5 was used to classify footprints by enrichment. Red, enriched cluster; blue, non-enriched cluster.

FIG. 14|Identification of TAL1/GATA1 complex dysregulation in mIDH2 AML. a, Comparison of enriched sequence-specific transcription factors between iDAPT-seq bivariate footprint analysis and iDAPT-MS. b, Comparison of enriched chromatin-associated proteins with K562 ChIP-seq profiles from ENCODE in iDAPT-seq by gene set enrichment analysis and iDAPT-MS. c, Comparison of BioGrid protein interaction network enrichment in iDAPT-MS by gene set enrichment analysis and iDAPT-MS. d, Western blot of TAL1 across TF1 pLVX cell lines. HSP90 is used as loading control. e, Enrichment analysis of TAL1 ENCODE K562 ChIP-seq peaks within both GATA1 ENCODE K562 ChIP-seq peaks and either differentially inaccessible (log 2 fold change <0 and FDR >5%) or accessible (log 2 fold change >0) iDAPT-seq peaks in the mIDH2 setting. Chi-squared test p-value is reported inline. f, Gene set enrichment analysis of genes proximal to closed GATA1/TAL1 binding sites. Genes from transcriptome profiles of TCGA AML patient samples (mIDH1/2 versus wild type IDH1/2) are ranked by signed −log 10 p-value by DESeq2. g, Western blot of corresponding TF1 cell lines. HSP90 is used as loading control. h, LC-MS/MS metabolite profiling of intracellular 2HG levels (mean±s.d.; n=3 repeatedly measured samples for each cell line). Two-tailed t-test was performed. i, GM-CSF-independent TF1 proliferation assessment (mean±s.d.; n=4 repeatedly measured samples for each cell line). Linear regression of normalized luminescence values was performed using sample type and day as categorical variables with interaction between the two variables (luminescence˜sample+day+sample:day). Reported p-values were from the interaction of sample type with day 13, with TF1 parental cell line as baseline. j, Representative gating strategy for flow cytometry analyses. k, Representative flow cytometry plots of TF1 parental or IDH2^{R140Q} knock-in cells, cultured either with erythropoietin and hemin chloride or normally with GM-CSF (n=1). l, Western blot of TAL1 across TF1 IDH2^{R140Q} knock-in cell lines transduced with pSIN4 constructs. HSP90 is used as loading control. m, LC-MS/MS metabolite profiling of intracellular 2HG levels (mean±s.d.; n=3 repeatedly measured samples for each cell line). Two-tailed t-test was performed. n, GM-CSF-independent TF1 IDH2^{R140Q} knock-in cell proliferation assessment (mean±s.d.; n=4 repeatedly measured samples for each cell line). Linear regression of normalized luminescence values was performed using sample type and day as categorical variables with interaction between the two variables (luminescence˜sample+day+sample:day). Reported p-values were from the interaction of sample type with day 13, with TF1 IDH2^{R140Q} knock-in transduced with pSIN4 empty vector (EV) as baseline.

FIG. 15|(a) Fragment size distributions of GM12878 ATACseq/iDAPT-seq libraries. (b) Ratio of transposon insertions at Ensembl v94 transcription start sites (TSS) relative to background from in-house ATAC-seq/iDAPTseq and published ATAC-seq libraries generated from the GM12878 cell line (n=1). (c) Proportion of non-mitochondrial reads from GM12878 ATAC-seq/iDAPT-seq libraries. (d) Heatmap of pairwise Pearson correlation coefficients of genome-wide transposon insertion frequencies for the indicated GM12878 ATAC-seq/iDAPT-seq libraries

FIG. 16|Assessment of peroxidase activity of transposase/peroxidase (TP) fusion probes. (a) Western blot of relative purified enzyme inputs (FLAG M2). The image is representative of two independent experiments. (b) Peroxidase activity assessment of purified recombinant enzymes measured by Amplex UltraRed fluorescence in the presence of 1 mM hydrogen peroxide for one minute (mean±s.e.m.; n=5 distinct samples for each condition, single protein purification batch per enzyme). Pairwise two-tailed ttests with pooled variance were performed, using Holm p-value adjustment to control for family-wise error rate. (c) Crystal structure of dimeric Tn5 transposase from ref. 23 (PDB: 1MUH). Visualization was performed using Mol.

FIG. 17|Optimization of iDAPT protein labeling in the HEK293T cell line. (a) Schematic of iDAPT protein labeling, with points of protocol optimization demarcated. (b and c) Western blot of labeled nuclear lysates with varying numbers of post-transposition washes (b) and buffer adjustments (c).

Images are representative of two independent experiments. Ratios, relative total streptavidin intensities normalized by corresponding PCNA intensities. T, Tn5-F; A, APEX2-F. LT, lysis and transposition.

FIG. 18|(a) Western blot of labeled nuclear lysates with negative (Tn5-F, APEX2-F) and fusion (TP1-5) probes. Images are representative of two independent experiments. Ratios, relative total streptavidin intensities normalized by corresponding PCNA intensities. (b) Western blot of labeled nuclear lysates with either single enzymatic domains (T, Tn5-F; A, APEX2-F) or the TP3 fusion probe with or without either biotin-phenol or hydrogen peroxide (H2O2). Images are representative of two independent experiments. Ratios, relative total streptavidin intensities normalized by corresponding PCNA intensities. (c) Heatmap of pairwise Pearson correlation coefficients of K562 iDAPT-MS profiles for the indicated probes. (d) Venn diagram of significant proteins (log 2 fold change >0 and false discovery rate <5%) identified by TP5 or TP3 versus negative control probes by iDAPT-MS

FIG. 19|iDAPT-MS reveals the open chromatin-associated proteome. (a) Schematic of iDAPT-MS experimental design and SL-TMT sample labeling for K562 profiling. (b) Volcano plot of proteins enriched by fusion (TP3 and TP5) versus negative control (Tn5-F and APEX2-F) probes in K562 nuclei. Blue points, log 2 fold change >0 and false discovery rate (FDR)<5%; red points, CisBP sequence-specific transcription factors; black points, points with corresponding gene symbol labels. (c) IGV genome track view of iDAPT-seq (TP3) libraries generated from either intact nuclei or genomic DNA from K562 cells and CUT&RUN libraries from K562 nuclei using ERH, WBP11, or normal rabbit IgG antibodies. (d) Representative images of co-immunofluorescence staining of the SC35 nuclear speckle marker with Tn5-F ATAC-see in the HT1080 cell line. Similar results were visually confirmed for more than ten nuclei for each chromatin marker and are quantified in FIG. 22c. Scale bars, 5 pm. (e and 0 Mediator (e) and BAF (f) CORUM complex enrichment by iDAPT-MS with fusion probes in both K562 and NB4 cell lines. NES (normalized enrichment score) and p-value, gene set enrichment analysis. Legend, individual protein-level iDAPT-MS enrichment. (g) MAX BioGrid first-order protein interaction network enrichment by iDAPT-MS with fusion probes in the K562 cell line. NES (normalized enrichment score) and p-value, gene set enrichment analysis. Legend, individual protein-level iDAPT-MS enrichment. (h) Distribution of Jaccard indices between MAX ChIP-seq peaks and ChIP-seq peaks of first-order protein interactors within regions of open chromatin in the K562 cell line. MAX ChIP 1, ENCFF618VMC. MAX ChIP 2, ENCFF900NVQ. BG, background ChIP-seq epitopes, collated from ENCODE K562 ChIP-seq datasets of proteins not annotated to interact with MAX by BioGrid. Center line, median value; box limits, upper and lower quartiles; whiskers, 1.5× interquartile range; black points, outliers. Red point, replicate MAX ChIP-seq epitope. p-values, two-sided Wilcoxon rank-sum test. n, number of represented ChIP-seq epitopes.

FIG. 20|(a) Western blot of labeled nuclear lysates with Tn5-F or TP3 probes and with or without pre-transposition blocking of endogenous peroxidase activity with 0.1% sodium azide and 0.03% hydrogen peroxide. Images are of a single experiment. Ratios, relative total streptavidin intensities normalized by corresponding PCNA intensities. (b) Schematic of iDAPT-MS experimental design and SL-TMT sample labeling for NB4 cell line profiling. (c) Volcano plot of proteins enriched by fusion (TP3) versus negative control (Tn5-F and APEX2-F) probes in NB4 nuclei. Blue points, log 2 fold change >0 and false discovery rate (FDR)<5%; red points, CisBP sequence-specific transcription factors; black points, points with corresponding gene symbol labels. (d) Heatmap of pairwise Pearson correlation coefficients of NB4 iDAPT-MS profiles for the indicated probes and treatment conditions.

FIG. 21|(a) Scatterplot of protein enrichment profiles by iDAPTMS from both K562 and NB4 cell lines. (b and c) CUT&RUN (top) and immunoprecipitation (bottom) enrichment of ERH (b) and WBP11 (c) in K562 cells relative to normal rabbit IgG antibody. Western blotting images are of a single experiment. Red lines, CUT&RUN enrichment of target epitopes across K562 iDAPT-seq peaks. Black lines, CUT&RUN enrichment of normal rabbit IgG antibody across K562 iDAPT-seq peaks. Solid and dashed lines, duplicate CUT&RUN analyses. (d) Distribution of CUT&RUN peaks overlapping K562 iDAPT-seq peaks. CUT&RUN peaks were determined using a 1% false discovery rate cut-off from MACS2. (e) Number of iDAPT-seq peaks overlapping ChIP-seq peaks in K562 cells. Listed proteins are profiled in K562 cells by the ENCODE consortium and are enriched by K562 iDAPT-MS (5% FDR).

FIG. 22|(a and b) Subcellular enrichment of K562 (a) and NB4 (b) iDAPT-MS profiles, using annotations from the Human Protein Atlas. NES (normalized enrichment score) and FDR (false discovery rate), gene set enrichment analysis. (c) Distribution of Pearson correlation coefficients between Tn5-F ATAC-see and co-immunostaining of the SC35 nuclear speckle marker or chromatin state markers (RNA Pol II S2P, H3K27Ac) per nucleus in three cancer cell lines. Numbers of nuclei assessed per marker are displayed inline, with images drawn from two independent experiments. Center line, median value; box limits, upper and lower quartiles; whiskers, 1.5× interquartile range; points, outliers. (d and e) Representative images of co-immunofluorescence staining of the SC35 nuclear speckle marker with Tn5-F ATAC-see in the MDA-MB-231 (d) and the DU145 (e) cancer cell lines. Similar results were visually confirmed for more than ten nuclei for each cell line and are quantified in (c). Scale bars, 5 pm. (0 Proportion of annotated proteins detected and significantly enriched (log 2 fold change >0 and FDR <0.05) by iDAPT-MS for the given protein families. n, total number of proteins annotated in each protein family. (g) Distribution of iDAPT-MS log 2 fold changes of detected histone and non-histone proteins. Center line, median value; box limits, upper and lower quartiles; whiskers, 1.5× interquartile range; black points, outliers. n, number of quantified proteins by iDAPT-MS per group. p-value, twosided Wilcoxon rank-sum test with Bonferroni correction.

FIG. 23|Binary comparison of K562 iDAPT-MS profiles enriched via recombinant fusion and negative control probes. (a-f) Volcano plots of pairwise comparisons of K562 iDAPT-MS profiles from recombinant fusion and negative control probes. Red points, CisBP sequence-specific transcription factors. (g) Volcano plots of K562 iDAPT-MS profiles from fusion probes versus APEX2-F, with profiles subjected to either bait (streptavidin/trypsin) peptide normalization or quantile normalization. Red points, CisBP sequencespecific transcription factors. (h) Subcellular enrichment of quantile-normalized K562 iDAPT-MS profiles as in (g), using annotations from the Human Protein Atlas. NES (normalized enrichment score) and FDR (false discovery rate), gene set enrichment analysis.

FIG. 24|Analysis of published open chromatin proteome enrichment by iDAPT-MS. (a and b) Fraction of proteins detected or enriched (a) and differences in proportions relative to RNA-seq (b) of K562 iDAPT-MS, nuclear proteome, whole cell proteome, or RNA-seq datasets among annotated proteins by the Human Protein Atlas. (c and d) Fraction of proteins detected or enriched (c) and principal component analysis (d) of K562 iDAPT-MS and K562 differential salt extraction proteomic datasets among annotated proteins by the Human Protein Atlas. (e and f) Fraction of proteins detected or enriched (e) and principal component analysis (f) of iDAPT-MS and published differential MNase digestion or salt extraction proteomic datasets among annotated proteins by the Human Protein Atlas.

FIG. 25|Integrative analysis of iDAPT-MS and iDAPT-seq classifies transcription factor activities on open chromatin at steady state. (a) Enrichment of CisBP sequence-specific transcription factors via K562 iDAPT-MS. Normalized enrichment score (NES) and p-value, gene set enrichment analysis. (b) Schematic of bivariate footprinting analysis of iDAPT-seq data. FPD, footprint depth. FA, flanking accessibility. (c) Bivariate footprinting analysis of native chromatin versus naked genomic DNA from the K562 cell line. Red, class A transcription factors; blue, class B transcription factors; gray, class C transcription factors. (d-f) K562 genome-wide footprint of CTCF (d, class A), RELA/p65 (e, class B), and IKZF1 (f, class C) from native chromatin (red) and naked DNA (black). Corresponding iDAPT-MS and ENCODE ChIP-seq enrichment metrics are listed below. iDAPT-MS LFC, log 2 fold change; FDR, limma false discovery rate. ChIP-seq NES, normalized enrichment score; p, gene set enrichment analysis p-value. (g) Comparison of CisBP sequence-specific transcription factors enriched by iDAPT-MS versus iDAPT-seq footprinting analysis in the K562 cell line. (h) Number of significant CisBP transcription factors in each footprinting class as determined by iDAPT-MS or ENCODE ChIP-seq, with corresponding numbers of associated transcription factor motifs per class as determined by iDAPT-seq.

FIG. 26|(a) Enrichment of CisBP sequence-specific transcription factors via NB4 iDAPT-MS. Normalized enrichment score (NES) and p-value, gene set enrichment analysis. (b) Fragment size distributions of iDAPT-seq libraries generated from K562 and NB4 native chromatin and naked genomic DNA. (c and d) Ratio of transposon insertions at Ensembl v94 transcription start sites (TSS) relative to background from K562 (c) and NB4 (d) iDAPTseq datasets. (e and f) Principal component analysis of genome-wide transposon insertion frequencies from K562 (e) and NB4 (f) iDAPT-seq libraries. (g and h) Volcano plot of K562 (g) and NB4 (h) iDAPT-seq profiles analyzed with DESeq2. Peak statistics are listed below. FDR, false discovery rate; LFC, log 2 fold change.

FIG. 27|(a and b), Classification scheme of transcription factor motifs by composite footprinting score from K562 (a) or NB4 (b) iDAPT-seq datasets. Separation of class A and B motifs was determined by a two-state Gaussian mixture model; separation of class B and C motifs was demarcated by either a false discovery rate >5% or footprinting score <0. (c) Bivariate footprinting analysis of native chromatin versus naked genomic DNA from the NB4 cell line. Red, class A transcription factors; blue, class B transcription factors; gray, class C transcription factors. (d) Tabulation of transcription factor footprinting classifications for those transcription factors significantly enriched by both K562 and NB4 iDAPT-MS. (e) Comparison of CisBP sequence-specific transcription factors enriched by fusion probe iDAPT-MS versus iDAPT-seq footprinting analysis in the NB4 cell line.

FIG. 28|iDAPT profiling of the NB4 acute promyelocytic leukemia cell line upon all-trans retinoic acid (ATRA) treatment reveals dynamics of transcription factor activity. (a) Schematic of the consequences of PML-RARA fusion oncogene on hematopoiesis and relief of its differentiation blockade by ATRA treatment. (b) Representative flow cytometry plots of NB4 cells treated with or without ATRA after 48 hours. (c) Comparison of CisBP sequence-specific transcription factor enrichment by TP3 iDAPT-MS (log 2 fold change) versus iDAPT-seq footprinting analysis (composite footprinting score) in the NB4 cell line upon treatment with either ATRA or DMSO. Roman numerals, transcription factor classification as described in FIG. 33a. (d-g) PU.1/SPI1 and BCL11A BioGrid first-order protein interaction networks (d and f) and corresponding genome-wide motif footprints (e and g) upon treatment with either ATRA (red) or DMSO (black) in the NB4 cell line. NES (normalized enrichment score) and p-value, gene set enrichment analysis. Legend, individual protein-level iDAPT-MS enrichment. (h) Assessment of NB4 cell line-specific genetic dependencies versus NB4 iDAPT-MS negative enrichment upon ATRA treatment. Dependency scores are as reported from the CRISPR (Avana) 19Q3 dataset.

FIG. 29|(a) Representative gating strategy for flow cytometry analyses as in FIG. 28b. (b) Western blotting analysis of the PML epitope from the NB4 cell line upon 48 hours ATRA treatment versus DMSO vehicle treatment (0.01%). Images are representative of two independent experiments. PCNA, loading control. (c) NB4 cell counts after 48 hours of treatment with either 1 μM ATRA or vehicle (0.01% DMSO), as measured by CellTiter-Glo (n=5 independent wells). p-value, Welch two-tailed t-test. (d) Volcano plot of proteins enriched by the TP3 fusion probe in NB4 nuclei treated with either ATRA or DMSO. Blue points, log 2 fold change >0 and false discovery rate (FDR)<5%; red points, log 2 fold change <0 and false discovery rate (FDR)<5%; black points, points with corresponding gene symbol labels. (e) ReactomeDB pathway enrichment analysis from iDAPT-MS of NB4 ATRA versus DMSO treatment. FDR, gene set enrichment analysis false discovery rate.

FIG. 30|Analysis of NB4 iDAPT-seq profiles upon treatment with ATRA. (a) Volcano plot of NB4 iDAPT-seq profiles upon either ATRA or DMSO treatment as analyzed with DESeq2. Peak statistics are listed below. FDR, false discovery rate; LFC, log 2 fold change. (b) Bivariate footprinting analysis of iDAPT-seq from the NB4 cell line treated with ATRA versus DMSO. R, Pearson correlation coefficient. (c) Distribution of composite footprinting scores from NB4 ATRA versus DMSO iDAPT-seq datasets. Thresholds were assigned based on false discovery rate <5%. (d-e) Scatterplots of flanking accessibility (d) and footprint depth (e) versus composite footprinting score. R, Pearson correlation coefficient.

FIG. 31|Assessment of iDAPT-seq footprinting versus motif enrichment analyses upon NB4 treatment with ATRA. (a) Principal component analysis of ChromVAR motif enrichment scores from iDAPT-seq profiles of ATRA- and DMSO-treated NB4 cells (b) Scatterplot of signed −log 10 false discovery rates (FDR) of ChromVAR motif enrichment versus composite footprinting scores from iDAPT-seq upon ATRA treatment in the NB4 cell line. R, Pearson correlation coefficient. (c) Comparison of CisBP sequence specific transcription factor enrichment by iDAPT-MS (log 2 fold change) versus ChromVAR motif enrichment (signed −log 10 FDR) in the NB4 cell line upon treatment with either ATRA or DMSO.

FIG. 32|Assessment of iDAPT-MS versus RNA-seq datasets upon NB4 treatment with ATRA. (a) Principal component analysis of publicly available RNA-seq profiles of ATRA- and DMSO-treated NB4 cells (GSM1288651, GSM1288652, GSM1288653, GSM1288654, GSM1288659, GSM1288660, GSM1288661, GSM1288662, GSM2464389, GSM2464392). (b) Scatterplot of log 2 fold changes of protein abundances versus transcript abundances from iDAPT-MS and RNA-seq, respectively, upon ATRA treatment in the NB4 cell line. R, Pearson correlation coefficient. (c) Comparison of CisBP sequence-specific transcription factor enrichment by RNA-seq (log 2 fold change) versus iDAPT-seq footprinting analysis (composite footprinting score) in the NB4 cell line upon treatment with either ATRA or DMSO.

FIG. 33|(a) Schematic outlining the nine classes emerging from the changes in transcription factor abundances and activities on open chromatin upon ATRA treatment. Concordant or discordant changes in abundance and activities suggest activating or repressive activities on chromatin, respectively. (b) Distribution of log 2 fold changes of transcription factor abundances as enriched by TP3 versus negative control iDAPT-MS profiles from untreated NB4 cells, separated by repressive (class I, increasing chromatin accessibility, decreasing protein abundance) or activating (class VII, decreasing chromatin accessibility, decreasing protein abundance) transcription factors as classified upon NB4 treatment with ATRA (mean±s.e.m.). n, number of represented proteins from NB4 iDAPT-MS. p-value, two-sided Wilcoxon rank-sum test.

FIG. 34|Integrative analysis of representative transcription factor abundances, activities, and protein complex dynamics. (a-c) Inference of transcription factor complex dynamics (top) and footprinting activities (bottom) of representative class I (a), class VII (b), and class IX (c) transcription factors upon treatment with either ATRA (red line) or DMSO (black line) in the NB4 cell line. Legend, individual protein-level iDAPT-MS enrichment.

FIG. 35|Integration of genetic dependency maps and iDAPT datasets. (a) Distribution of genetic dependency scores across all hematopoietic cancer cell lines assayed in the CRISPR (Avana) 19Q3 dataset. The DepMap score threshold for hematopoietic cell line dependency was determined by a two-state Gaussian mixture model. (b) Distribution of the number of cancer cell lines dependent on a given gene as determined in (a). Genes classified as dependencies in at least half of all hematopoietic cell lines were demarcated as essential genes. (c-d) Inference of transcription factor complex dynamics (top) and footprinting activities (bottom) of ZEB2 (c) and EBF3 (d) upon treatment with either ATRA (red line) or DMSO (black line) in the NB4 cell line. Cognate sequence motifs are displayed above the corresponding footprinting profiles. Legend, individual protein-level iDAPT-MS enrichment.

FIG. 36|Analysis of PU.1/SPI1 transcription factor complex dynamics inferred by iDAPT-MS versus RNA-seq. PU.1/SPI1 BioGrid first-order protein interaction network enrichment by iDAPTMS (left) or RNA-seq (right) in the NB4 cell line upon treatment with ATRA. NES (normalized enrichment score) and p-value, gene set enrichment analysis. Legend, individual protein-level iDAPT-MS or transcript level RNA-seq enrichment.

### DETAILED DESCRIPTION

The invention provides compositions and methods for facilitating direct, unbiased identification of genomic sequences and corresponding proteome and/or transcriptome components at sites of open chromatin. As explained further below, the methods of the invention employ fusion proteins that include a first enzyme that fragments and tags accessible genomic DNA and a second enzyme that labels molecules (e.g., proteins, peptides, and/or RNA) that are proximal to the accessible genomic DNA. The tagged and labeled molecules can then be identified in order to generate a profile characteristic of the region of open chromatin and the cell from which they were obtained.

The invention can be used in a wide range of contexts. For example, interrogation of open chromatin according to the invention can be used to characterize and identify chromatin features associated with disease states, responses to biological or chemical treatment or other stimuli, as well as different stages development. Through the methods of the invention, a user is able to identify genomic regulatory positions, sequence-specific transcription factors with long and short retention times on DNA, and additional associated proteins and other molecules across accessible chromatin. Furthermore, transcription factor gene targets and their protein complex components can be inferred in order to obtain a complete portrait of cis-regulation within a cell. The methods do not require genetic manipulation of biological samples of interest, and thus may be readily applied to numerous biological materials, including patient samples, to uncover molecular pathologies underpinning disease states. The invention can thus be used to unravel epigenomic landscapes underpinning normal development and disease states in both model systems and in patient-derived samples.

The compositions and methods of the invention are described further, as follows.

### Fusion Proteins

As noted above, the fusion proteins of the invention include a first enzyme that fragments and tags accessible genomic DNA and a second enzyme that labels molecules (e.g., proteins, peptides, RNA, or carbohydrates) that are proximal to the accessible genomic DNA. The enzyme components of the fusion proteins can be present in the molecules in either order. Thus, for example, the first enzyme can be located in the amino terminal end of the fusion protein, while the second enzyme is located in the carboxyl terminal end of the fusion protein. Alternatively, the second enzyme can be located in the amino terminal end of the fusion protein, while the first enzyme is located in the carboxyl terminal end. Furthermore, the first and second enzymes of the fusion proteins can optionally be separated from one another by a linker sequence. Optionally, the fusion proteins can also include additional sequences. For example, the fusion proteins can optionally include tags that can be used, e.g., in purification or identification of the fusion proteins.

The first enzyme of the fusion proteins of the invention can be any enzyme that is capable of fragmenting and tagging a polynucleotide, such as genomic DNA. The first enzyme typically acts with minimal or no sequence specificity, thus fragmenting and tagging a polynucleotide, such as genomic DNA, based only on accessibility of the polynucleotide to the first enzyme. However, enzymes with sequence specificity, such as restriction enzymes, can also be used as first enzymes according to the invention.

Examples of enzymes that can be used as first enzymes, according to the invention, include transposases (e.g., Tn transposases, hAT transposases (e.g., Hermes transposase), and DD[E/D] transposases (e.g., SB transposase)), retroviral integrases (e.g., HIV integrase), and other DNA-binding enzymes, such as, e.g., DNase, MNase, and restriction enzymes. Specific, non-limiting examples of first enzymes include Tn transposases (e.g., Tn3, Tn5, Tn7, Tn10, Tn552, Tn903, Tn/O, and TnA), MuA transposases, Vibhar transposases (e.g., from Vibrio harveyi), Ac-Ds, Ascot-1, Bsl, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tc1, Tc3, Tel, THE-1, Toll, To 12, Ty1, and fragments, analogs, or variants thereof. Tn5 transposase (see, e.g., Picelli et al., Genome Res. 24:2033-2040, 2014; SEQ ID NOs: 1 and 2) is used in certain fusion proteins described further herein. Variants of Tn5 transposase can also be used in the invention. For example, engineered Tn5 super-mutants (e.g., TN5-059) can be used (see, e.g., Sos et al., Genome Biol. 17:20, 2016; Kia et al., BMC Biotech. 17:6, 2017).

In addition to the above-noted enzymes, fragments, analogs, and variants of the enzymes, and other enzymes having the requisite activity (i.e., fragmenting and tagging of DNA), can be used in the invention, provided that they maintain sufficient activity (i.e., fragmenting and tagging of DNA). Thus, for example, enzyme variants that maintain fragmenting and tagging activity, and have at least about 70%, 75%, 80%, 85%, 90%, 92%, 94%, 95%, 97%, 98%, or 99% amino acid sequence identity to a transposase, integrase, or other DNA-binding enzyme, e.g., an exemplary first enzyme listed above, or a fragment thereof (e.g., a fragment of at least about 15, 20, 30, 40, 50, 60, 75, 100, 150, 200, 250, 300, 350, 400, 450, 500, 550, or 600 amino acids in length), can be used. Also included are variants having one or more (e.g., 1, 2, 3, 4, 5, 6, 7, 8, 9, 10, 11, 12, 13, 14, 15, 16, 17, 18, 19, 20, or more amino acid substitutions or deletions, provided that they maintain sufficient activity. Moreover, the variant sequences can be present in the enzymes or in tag and/or linker sequences, as described herein.

The second enzyme of the fusion proteins of the invention can be any enzyme that is capable of labeling molecules (e.g., proteins, peptides, RNAs, or carbohydrates) that are proximal to a polynucleotide, such as genomic DNA. Although the second enzymes may, in some instances, react with some molecules or portions thereof preferentially as compared to others, due to for example the chemical make-up of the molecules (e.g., electron richness of a particular amino acid component), in general, the second enzymes are non-specific and label most molecules to which they are proximal, for example, when activated in the presence of a tagging substrate.

Examples of enzymes that can be used as a second enzyme, according to the invention, include peroxidases, biotin ligases, catalase-peroxidase enzymes (e.g., KatG), and oxidases (e.g., CueO and bilirubin oxidase). In addition to wild type versions of these enzymes, certain mutant forms of the enzymes can be used due to advantageous features of the mutants. For example, mutant forms of certain enzymes have increased activity or decreased specificity.

Examples of peroxidases that can be used in the invention include ascorbate peroxidase (APX), horseradish peroxidase (HRP; see, e.g., Bar et al., Nat. Methods 15(2):127-133, 2018), soybean ascorbate peroxidase, pea ascorbate peroxidase, Arabidopsis ascorbate peroxidase, maize ascorbate peroxidase, cytochrome c peroxidase, laccase, tyrosinase, and mutant forms thereof. Specific examples of ascorbate peroxidases (APXs) that can be used in the invention include APEX (see, e.g., Rhee et al., Science 339(6125):1328-1331, 2013; SEQ ID NO: 5) and APEX2 (see, e.g., Lam et al., Nature Methods 12:51-54, 2015; SEQ ID NOs: 3 and 4), the latter of which includes an A134P mutation relative to APEX.

Examples of biotin ligases that can be used in the invention include BirA and mutant forms thereof. For example, E. coli BirA can be used, which optionally includes a mutation in its active site (e.g., R118G; BiolD; Choi-Rhee et al., Protein Sci. 13:3043-3050, 2004) to facilitate non-specific labeling. As another example, a modified form of BirA from Aquifex aeolicus can be used, which optionally includes a mutation in its active site (e.g., R40G) (BiolD2; Choi-Rhee et al., supra; Kim et al., Mol. Biol. Cell 27:1188-1196, 2016; also see, e.g., Chen et al., Wiley Interdiscip. Rev. Dev. Biol. 6(4) 2017). Additional mutants of biotin ligase that can be used as second enzymes in the invention are TurboID and miniTurbo (Branon et al., Nat. Biotechnol. 36(9):880-887, 2018).

In addition to the above-noted enzymes, fragments, analogs, and variants of the enzymes, and other enzymes having the requisite activity (i.e., proximity labeling of molecules such as proteins, peptides, RNA, and/or carbohydrates), can be used in the invention, provided that they maintain sufficient activity. Thus, for example, enzyme variants that maintain proximity labeling activity, and have at least about 70%, 75%, 80%, 85%, 90%, 92%, 94%, 95%, 97%, 98%, or 99% amino acid sequence identity to a second enzyme, e.g., an exemplary second enzyme listed above, or a fragment thereof (e.g., a fragment of at least about 15, 20, 30, 40, 50, 60, 75, 100, 150, 200, 250, 300, 350, 400, 450, 500, 550, or 600 amino acids in length), can be used. Also included are variants having one or more (e.g., 1, 2, 3, 4, 5, 6, 7, 8, 9, 10, 11, 12, 13, 14, 15, 16, 17, 18, 19, 20, or more amino acid substitutions or deletions, provided that they maintain sufficient activity.

As noted above, the first and second enzymes of the fusion proteins of the invention can optionally be separated from one another by a linker. Approaches for selection of linkers for fusion proteins are known in the art (see, e.g., Chen et al., Adv. Drug Deliv. Rev 65(1):1357-1369, 2013). The structure of a linker that can be used in the invention is not particularly limited and can be, for example, a short or long peptide (e.g., 3-100, 5-75, 10-50, or 15-25 amino acids). The linker can optionally be rigid. For example, a helical peptide linker including one or more EAAAK (SEQ ID NO: 32) motif (e.g., AEAAAKEAAAKA (SEQ ID NO: 33)), or a proline-rich linker (e.g., PAPAP or (XP)n, where X is Ala, Lys, or Glu, and n is, e.g., 1, 2, 3, 4, 5, 6, 7, 8, 9, 10, 11, 12, 13, 14, 0115), can be used. Alternatively, a flexible linker can be used. Flexible linkers typically include small, non-polar (e.g., Gly) or polar (e.g., Ser or Thr) amino acids. Examples of such linkers include GS linkers, e.g., linkers of the structure (GGGGS)n, where n is, e.g., 1, 2, 3, 4, 5, 6, 7, 8, 9, 10, 11, 12, 13, 14, or 15 (SEQ ID NO: 34). In one example, n is 4. Additional examples are Gly8 and Gly6 linkers. Specific examples of linkers include the following: PAPAP (SEQ ID NO: 7), AEAAAKEAAAKA (SEQ ID NO: 9), (GGGGS)₄ (SEQ ID NO: 11), and GSGAGA (SEQ ID NO: 13). Variants of linker sequences can also be used, which include one or more (e.g., 1, 2, 3, 4, 5, 6, 7, 8, 9, 10, 11, 12, 13, 14, 15, 16, 17, 18, 19, 20, or more amino acid substitutions or deletions). Preferably, such changes do not substantially reduce (e.g., reduce by 20%, 30%, 40%, 50%, 60%, 70%, 80% or more) activity of the fusion protein, as compared to a corresponding non-variant sequence.

The invention also includes split enzymes and their use in the methods described herein. In one example of such split molecules, a first enzyme (e.g., a transposase, such as Tn5 transposase; also see other examples herein) is in its full-length form, but a second enzyme (e.g., a peroxidase, such as APEX or APEX2; also see other examples herein) to which the first enzyme is fused (e.g., as described herein) is split. Accordingly, the first enzyme is fused with a first portion (e.g., about half) of the second enzyme and, in a separate molecule, the first enzyme is fused with a second portion (e.g., the remaining half) of the second enzyme. In one example, a first molecule [transposase]-[peroxidase half #1] is used with a second molecule [transposase]-[peroxidase half #2] to form dimers, as Tn5 transposase normally does (form a 1:1 mixture of these two proteins). In another example, the first fusion is added first and then the second fusion is added after washing in order to initiate labeling.

In addition to the components noted above, the fusion proteins of the invention can also optionally include a tag or label that can be used, e.g., to facilitate purification of the proteins. Thus, for example, the fusion proteins can optionally include one or more peptide or protein tags. As one example, the proteins can optionally include a FLAG tag (e.g., DYKDDDDK; SEQ ID NO: 15), or a variant thereof (e.g., DYKDHD-G-DYKDHD-I-DYKDDDDK; SEQ ID NO: 16). In another example, a human influenza hemagglutinin or HA tag may be used (e.g., YPYDVPDYA; SEQ ID NO: 17). In other examples, an epitope tag (e.g., V5-tag, Myc-tag, HA-tag, Spot-tag, or NE-tag) or an affinity tag (e.g., chitin binding protein (CBP), maltose binding protein (MBP), Strep-tag, glutathione-S-transferase (GST), or poly(His) tag) can be used. The tags are typically located at the C-terminal end or the N-terminal end of the fusion proteins, but can be located anywhere within the proteins (e.g., between the enzymes or elsewhere within the fusion protein), provided that the desired activities of the proteins (fragmenting, tagging, or labeling) are maintained.

Several examples of fusion proteins of the invention include Tn5 (SEQ ID NO: 2) and APEX2 (SEQ ID NO: 4) sequences. The Tn5 and APEX2 components can be in either order and can optionally be separated from one another by a linker sequence. Further, the fusion proteins can optionally include a tag (e.g., a Flag tag). Thus, specific examples of fusion proteins of the invention include the following:

1. N-Tn5 (SEQ ID NO: 2)—APEX2 (SEQ ID NO: 4)—C

2. N-APEX2 (SEQ ID NO: 4)—Tn5 (SEQ ID NO: 2)—C

3. N-Tn5 (SEQ ID NO: 2)—Linker-APEX2 (SEQ ID NO: 4)—C, wherein the linker is selected from SEQ ID NOs: 7, 9, 11, and 13.

4. N-APEX2 (SEQ ID NO: 4)—Linker-Tn5 (SEQ ID NO: 2)—C, wherein the linker is selected from SEQ ID NOs: 7, 9, 11, and 13.

5. N-Tn5 (SEQ ID NO: 2)—Linker-APEX2 (SEQ ID NO: 4)—Tag-C, wherein the linker is selected from SEQ ID NOs: 7, 9, 11, and 13, and the Tag is selected from SEQ ID NOs: 15, 16, and 17.

6. N-APEX2 (SEQ ID NO: 4)—Linker-Tn5 (SEQ ID NO: 2)—Tag-C, wherein the linker is selected from SEQ ID NOs: 7, 9, 11, and 13, and the Tag is selected from SEQ ID NOs: 15, 16, and 17.

7. N-Tag-Tn5 (SEQ ID NO: 2)—Linker-APEX2 (SEQ ID NO: 4)—C, wherein the linker is selected from SEQ ID NOs: 7, 9, 11, and 13, and the Tag is selected from SEQ ID NOs: 15, 16, and 17.

8. N-Tag-APEX2 (SEQ ID NO: 4)—Linker-Tn5 (SEQ ID NO: 2)—C, wherein the linker is selected from SEQ ID NOs: 7, 9, 11, and 13 (or includes a motif of SEQ ID NO: 32, 33, or 34), and the Tag is selected from SEQ ID NOs: 15, 16, and 17.

In any of the above-listed examples, the first enzyme (Tn5) can be replaced with another first enzyme, such as one of the first enzyme examples described herein or another sequence known in the art; the second enzyme (APEX2) can be replaced with another second enzyme, such as one of the second enzymes described herein or another sequence known in the art; the linker can be present or absent and, if present, can be replaced with a different sequence, such as a different linker sequence described herein or known in the art; and the tag sequence can be present or absent and, if present, can be replaced with a different sequence, such as a different linker sequence described herein or known in the art.

Fusion proteins can be made using any of a number of standard methods that are known in the art. For example, the fusion proteins can be expressed in and purified from cells (e.g., bacterial cells, such as E. coli) that have been engineered to stably or transiently express the fusion proteins (see, e.g., Picelli et al., Genome Res. 24:2033-2040, 2014). Alternatively, the fusion proteins can be generated by standard peptide synthesis methods.

### Methods

The methods of the invention include contacting a polynucleotide, such as genomic DNA, with a fusion protein as described herein under conditions in which the first enzyme of the fusion protein fragments and tags accessible DNA in regions of open chromatin, and under conditions in which the second enzyme of the fusion protein labels molecules (e.g., proteins, peptides, RNA, or carbohydrates) that are proximal to the open chromatin. Then the tagged polynucleotide fragments and the labeled proximal molecules are characterized and identified in order to provide information regarding molecules that are present at the sites of open chromatin.

Chromatin that can be subject to analysis using the methods of the invention can be present in or isolated from cells including, for example, cells characteristic of a disease, condition, or developmental state of interest, or cells that have been treated with a particular molecule (e.g., a candidate therapeutic agent) or genetically modified (e.g., to create a disease model). The cells can be obtained from a patient having or suspected of having a disease or condition of interest, for use in diagnosis or monitoring effects of treatment. For example, the cells can be obtained from a tissue (e.g., a tumor) biopsy or from a blood sample. Alternatively, the cells can be cultured cell lines. The cells can optionally be modified to express a transgene or altered so that expression of an endogenous gene of interest is modified (e.g., increased, decreased, or knocked-out). The cells can further optionally be cultured under conditions that are associated with a particular phenotype with respect to which it is of interest to characterize changes in open chromatin. Thus, for example, the cells can be cultured in the presence of an additive (e.g., a drug, a nutrient, a receptor ligand, or another cell) or under varying conditions (e.g., temperature, medium components, etc.). Furthermore, the cells can optionally be selected for use in the methods of the invention by, e.g., phenotypic analysis. For example, the cells can be analyzed using fluorescence activated cell sorting (FACS) and/or laser capture microdissection (LCM). Additional information and examples of cells that can be used in the methods of the invention are provided below.

In the case of isolated chromatin, the chromatin used in the methods of the invention can be obtained using any suitable method. For example, cells can be lysed and nuclei isolated from the resulting lysate by, e.g., pelleting. Chromatin can further optionally be purified away from any remaining nuclear envelope. In some examples, chromatin is isolated by contacting isolated nuclei with a reaction buffer, which can include a fusion polypeptide as described herein, together with any required reagents (e.g., tags or labels). Also see, e.g., the methods described in the examples set forth below, as well as, e.g., Kuznetsov et al., J. Biol. Chem. 293:12271-12282, 2018; and Arrigoni et al., Nucl. Acids Res. 44(7):e67, 2016. In addition, kits that are commercially available for isolating chromatin (e.g., Chromatin Extraction Kit (ab117152, Abcam) or ChromaFlash Chromatin Extraction Kit, EpiGentek) can be used.

The number of cells needed as a source of chromatin used in the methods of the invention can be small, which can be particularly advantageous when the methods are used, for example, in characterizing open chromatin obtained from cells from patient samples or engineered cells. Thus, the number of cells used to obtain chromatin for use in the methods of the invention can be, e.g., about 100 to about 10⁶ or more cells, about 500 to about 100,000 cells, about 500 to about 50,000 cells, about 500 to about 10,000 cells, about 50 to 1000 cells.

Once a chromatin sample is obtained for use in the methods of the invention, it is incubated with a fusion protein as described herein under conditions appropriate for fragmenting and tagging of accessible genomic DNA by the first enzyme of the fusion protein, and labeling of proximal molecules (e.g., proteins, peptides, RNA, and carbohydrates) by the second enzyme of the fusion protein. These processes (fragmenting/tagging and labeling) can take place in either order or at the same time. In one example, fragmenting and tagging takes place first, and then after a sample of the reaction mixture is removed for analysis of fragmented and tagged DNA, labeling of proximal molecules takes place. The reactions can be carried out in, for example, standard micro-centrifuge tubes, the wells of a multi-well plate, or channels of, e.g., microfluidic cell culture systems.

The conditions used for the two reactions (fragmenting/tagging and labeling) can be selected by those of skill in the art depending upon, for example, the particular enzymes that make up a fusion protein that is being used. Thus, for example, if the first enzyme of the fusion protein is a Tn transposase (e.g., Tn5 transposase or a related enzyme), then methods such as those described in the following documents can be used or adapted for use in the invention: Corces et al., Nat. Methods 14:959-962, 2017; Picelli et al., Genome Res. 24:2033-2040, 2014; WO 2014/189957; Caruccio Methods Mol. Biol. 733:241-255, 2011; Kaper et al., Proc. Natl. Acad. Sci. U.S.A. 110:5552-5557, 2013; Marine et al., Appl. Environ. Microbiol. 77:8071-8079, 2011; US 2010/0120098; WO 2017/156336. In addition to the chromatin and fusion proteins (as well as standard buffers (e.g., DMF, e.g., 16% DMF, salts etc.), the reaction mixtures can also include tags for labeling fragmented genomic DNA. These tags are optionally adaptor molecules that can be used to facilitate sequencing, amplification, and/or library preparation. As an example, Tn5 can be assembled into a transposome with pre-annealed Mosaic End double-stranded oligonucleotides (MEDS-A/B), for use in a fragmenting/tagging reaction (see, e.g., Picelli et al., supra; Corces et al., supra; and WO 2012/103545). Sequences of oligonucleotides for use with particular sequencing platforms (e.g., Illumina) are known in the art and can be adapted for use in the invention (see, e.g., Picelli et al., supra; Corces et al., supra; and WO 2012/103545). Commercially available kits can optionally be used or adapted for use in the invention (e.g., Nextera™ or Nextera XT DNA sample preparation kits; Illumina). Additional tags that can be used in the invention include, e.g., polynucleotide tags (e.g., sequencing adaptors, locked nucleic acids (LNAs), zip nucleic acids (ZNAs), or RNAs), affinity reactive molecules (e.g., biotin), click chemistry handles, azides, alkynes, and phosphines (e.g., azide or alkene groups). Furthermore, the tags can also optionally include barcode labels for use in, e.g., facilitating multiplex sequencing and the identification of individual insertion events. Additionally, the tags can optionally be labeled for detection, e.g., by including fluorescent tags. Optionally, a portion of the reaction mixture, designated for DNA or RNA sequence analysis, can be treated with a protease prior to further processing.

After open chromatin has been fragmented and tagged to produce tagged fragments of genomic DNA, then a DNA library can be extracted from the reaction mixture (or a portion thereof) and amplified by PCR (e.g., quantitative PCR; see, e.g., Buenrostro et al., Nat. Methods 10:1213-1218, 2013). Optionally, sequencing primer sites for next generation sequencing can be added to the fragments during amplification. Libraries can then be sequenced for identification of the genomic DNA at the sites of open chromatin using any of a number of methods known in the art. The fragments can be sequenced using any of a number of different methods that are known in the art. For example, the fragments can be sequenced using the reversible terminator method (Illumina), pyrosequencing (Roche), the sequencing by ligation platform (the SOLID platform; Life Technologies), or the Ion Torrent platform (Life Technologies). (Also see Margulies et al., Nature 437:376-380, 2005; Ronaghi et al., Analytical Biochemistry 242:84-89, 1996; Shendure et al., Science 309:1728-1732, 2005; Imelfort et al., Brief Bioinform. 10:609-618, 2009; Fox et al., Methods Mol. Biol. 553:79-108, 2009; Appleby et al., Methods Mol. Biol. 513:19-39, 2009; and Morozova et al., Genomics 92:255-264, 2008). The identified sequences can then be analyzed in comparison to sequence and motif databases, with filters (e.g., filters removing mitochondrial DNA sequences) optionally applied, as is known in the art.

As is the case with respect to the first enzyme of the fusion proteins of the invention, selection of conditions for activity of the second enzyme of the fusion proteins can be carried out by those of skill in the art, depending upon the nature of the second enzyme. In general, the second enzymes catalyze reactions in which a substrate is converted to a reactive form that labels nearby molecules, e.g., by the formation of a covalent bond. Thus, for example, if the second enzyme is, e.g., a peroxidase (e.g., APEX, APEX2, or HRP; also see above), then the labeling reaction can include the use of, e.g., hydrogen peroxide and a labeling molecule (e.g., biotin-tyramide/biotin-phenol, or biotin arylazide). In particular, peroxidases convert a substrate (e.g., biotin-tyramide/biotin-phenol, or biotin arylazide) to a short-lived, highly reactive radical under oxidizing conditions (e.g., exposure to H₂O₂). The radical then covalently attaches to electron-rich amino acids in nearby proteins. The labelling reaction can be stopped by removing H₂O₂ and quenching, and then the biotinylated proteins can be isolated using, e.g., streptavidin beads. Additional details regarding methods for tagging proximal molecules with, e.g., peroxidases are known in the art (see, e.g., U.S. Pat. No. 9,624,524) and can be used or adapted for use in the methods of the present invention.

In a variation of the above-described methods, RNA molecules are chemically cross-linked to proximal proteins and peptides using, e.g., formaldehyde (see, e.g., Kaewsapsak et al., eLIFE 6:229224, 2017). This can take place before, at the same time as, or after the labeling reaction of the second enzyme. Cross-linked RNA molecules are then optionally sheared and RNA libraries are analyzed by RNAseq. The identified sequences are then processed by, e.g., comparison to transcriptome databases, with filters optionally applied, leading to the generation of information regarding RNA molecules associated with open chromatin.

Isolated, labeled proteins and peptides are optionally fragmented (e.g., by trypsin digestion) and then are analyzed using techniques that are known in the art. These methods can include one or more of the following steps: labeling, fractionation, spectrometric detection (e.g., by mass spectroscopy (MS), e.g., LC-MS/MS; also see, e.g., Chen et al., Wiley Interdiscip. Rev. Dev. Biol. 6(4), 2017), and analysis in the context of sequence databases (e.g., proteomic or transcriptomic databases), with filters optionally applied. In one example, peptides are labeled by tandem mass tag (TMT) labeling using, e.g., the SL-TMT method (Navarette-Perea et al., J. Proteome Res. 17:226-2236, 2018). The TMT-labeled peptides are then pooled, and pooled samples are then fractionated using HPLC methods (e.g., off-line basic pH reversed-phase (BPRP) HPLC; Wang et al., Proteomics 11:2019-2026, 2011). Samples are then subject to synchronous precursor selection mass spectroscopy (SPS-MS) for peptide identification and quantitation. The resulting data can be processed in the context of available databases. For example, the data may be filtered so that, e.g., proteins from subcellular locations outside the nucleus are excluded. In addition, the data may be processed in connection with, e.g., transcription factor databases (e.g., CisBP; Weirauch et al., Cell 158:1431-1443, 2014).

A final data set of transcription factors and associated molecules (e.g., RNA molecules) that are identified can then be analyzed in the context of each other and the fragmented genomic sequence information, in order to capture interactions between various transcription factor components, and facilitating the inference of cis-regulatory transcription factor networks and their corresponding protein and RNA interactors. This analysis can be carried out in order to obtain a systemic overview of the epigenomic landscape. Thus, an epigenetic map of the open chromatin can be prepared (see, e.g., WO 2014/189957), and then integrated with information concerning proximal molecules, as described above.

### Use

As noted above, the compositions and methods of the invention can be used in a wide range of contexts. In particular, the methods can be used in any instances in which it is useful to obtain information as to the status of the composition of open chromatin of a cell. For example, the methods can be used to characterize and identify chromatin features associated with disease states, responses to biological or chemical treatment or other stimuli, physiological changes, as well as different periods of time (e.g., different stages development). The methods can thus be used to determine whether a subject has or is at risk of developing a disease or condition associated with an epigenomic change. The methods can further be used to determine a proper course of treatment for a patient, to track the course of treatment, to obtain guidance as to possible treatment changes, or to monitor a treated patient for possible relapse and/or to obtain guidance as to possible treatment changes. Additionally, the methods can be used to identify targets for drug development. For example, transcription factors can be identified that are associated with open chromatin including sequences regulating a gene that is active during a disease process. Such transcription factors can then serve as targets in drug (e.g., small molecule, antibody, dominant-negative, antisense, or RNAi) screens. The methods of the invention can be used to compare the cells of two or more different samples. This can be done, for example, with cells of a diseased tissue as compared to a corresponding healthy tissue. This also can be done with cells of a subject obtained from the same tissue at different times (e.g., before, during, or after treatment) or after exposure to different treatments (e.g., treatment with a drug). The methods can further be used to characterize, classify, grade, stage, diagnose, prognose, or assess risk of a disease or condition of a subject. Further, the methods of the invention can be used to gain insight into basic cellular processes in normal or diseased states. Additionally, the methods can be used to identify and characterize multiple transcription factors associated with open chromatin and, in monitoring how the composition of such a group of transcription factors changes in the context of open chromatin, in response to a stimulus (e.g., therapeutic treatment), physiological change, or over time, insight can be gained as to how the transcription factors function together. Thus, for example, abundance and/or activities of the transcription factors can be analyzed and the results integrated to obtain information as to how multiple transcription factors function in complex processes. Insight gained from such analyses can be used, for example, to identify targets, e.g., for therapeutic intervention, or to test candidate therapies. Furthermore, transcription factor networks can be identified and characterized with respect to the transcription factors and corresponding cis-acting sequences, and complex protein dynamics can be discerned.

Examples of diseases and conditions that can be subject to analysis using the methods of the invention include cancer, metastasis or recurrence of cancer, and other cell proliferative disorders, as well as diseases and conditions of metabolism, the immune system, the central nervous system (e.g., dementia, Parkinson's disease, Lewy body disease, and other neurodegenerative diseases and conditions), the cardiovascular system, the gastrointestinal tract, the respiratory system, the skin, the musculoskeletal system, connection tissues, endocrine system. The methods of the invention can further be used in the context of inflammation, autoimmunity, infectious disease, developmental disorders, trauma, and exposure to environmental hazards (e.g., toxins). The methods of the invention also can be used to identify open chromatin-associated molecules that are associated with resistance to treatment, thus providing targets for the development or use of different therapies.

The chromatin subject to analysis according to the methods of the invention can be obtained from any types of cells including, for example, cells that are characteristic of a disease, condition, or developmental state of interest (e.g., one or more of the diseases or conditions listed above). In some examples, the cells are obtained from a subject (e.g., a human subject) having or suspected of having a disease or condition of interest. The cells can be obtained from fresh, frozen, or fixed tissue samples, as well as from tissue explants or biopsies (e.g., tumor biopsies or biopsies of tissues infected with a pathogen). Examples of tissues from which cells can be obtained include soft tissues (e.g., brain, adrenal gland, skin, lung, spleen, kidney, liver, spleen, lymph node, bone marrow, bladder, stomach, small intestine, large intestine, or muscle). In some examples, the cells are obtained from a tumor or a tissue suspected of including cancerous cells (e.g., colon, breast, prostate, lung, or skin tissues). In addition to soft tissues, e.g., the soft tissues listed above, the cells can be obtained from body fluids including, e.g., blood, plasma, saliva, mucous, phlegm, cerebral spinal fluid, pleural fluid, tears, lactal duct fluid, lymph, sputum, cerebrospinal fluid, synovial fluid, urine, amniotic fluid, and semen. In regard to blood cells, the cells can be obtained from a sample of whole blood (e.g., peripheral blood) or a blood fraction. Examples of blood and related cells that can be subject to the methods of the invention include platelets, red blood cells, white blood cells (including, e.g., peripheral blood leukocytes, such as neutrophils, lymphocytes (e.g., T cells, B cells, and NK cells), eosinophils, basophils, and monocytes.

In addition to patient-derived cells, e.g., of the types described above, cell lines (e.g., immortalized cell lines) or other cultured cells can be the source of chromatin to be analyzed according to the methods of the invention. Thus, for example, cells that are induced to express a gene of interest can be used. The cells can be artificially induced to have a phenotype of interest by, e.g., altering gene expression in the cell. For example, a cell can be modified by to express a transgene of interest or may be knocked out or edited to remove a gene. Furthermore, the cells can be infected with a pathogen, or treated (e.g., with environmental or chemical agents, such as peptides, hormones, altered temperature, growth conditions, physical stress, pathogens, or drugs). The methods of the invention can be carried out using cells from, e.g., humans, non-human mammals (e.g., animal models, such as mice, rats, and non-human primates, as well as livestock animals), or cultured derivatives of these cells.

In certain cases, the cells that are analyzed according to the methods of the invention are also analyzed using different methods, before or after characterization according to the methods of the present invention. Thus, for example, the cells (or other cells of from the same source) can also be analyzed using fluorescence activated cell sorting (FACS), laser capture microdissection (LCM), or immunohistochemical methods.

### Kits

The invention also provides kits that can be used in carrying out the methods of the invention. The kits can include, for example, a fusion protein of the invention, such as one or more of the fusion proteins described above (e.g., a fusion protein containing Tn5 and APEX2, as described herein) or a nucleic acid molecule encoding such a fusion protein. The kits can also optionally include tags to label fragmented DNA (e.g., sequencing adaptors) and/or labels for proximity labeling of proteomic and/or transcriptomic components associated with open chromatin (e.g., biotin-phenol; also see above). The kits can further optionally include buffers (e.g., cell lysis buffers or reaction buffers). The different components of the kits can be present in separate containers within the kits, or certain compatible components can be pre-combined into single containers. In addition to above-mentioned components, the subject kits can also include instructions for using the components of the kits to practice the methods described herein.

The invention is illustrated in the following, non-limiting examples.

### EXAMPLES

### Example 1

### Introduction and Results

The architecture of chromatin accessibility regulates eukaryotic cell identity by controlling transcription factor access to regulatory sites and is frequently disrupted in disease (Kornberg et al., Annu. Rev. Cell Dev. Biol. 8:563-587, 1992; Gerstein et al., Nature 489:91-100, 2012; Lambert et al., Cell 172:650-665, 2018; Dann et al., Nature 548:607-611, 2017; Allis et al., Nat. Rev. Genet. 17:487-500, 2016; Klemm et al., Nat. Rev. Genet. 20:207-220, 2019; Thurman et al., Nature 489:75-82, 2012; Denny et al., Cell 166:328-342, 2016; Corces et al., Science 362 (6413), 2018). However, prior to the present invention, no biochemical approach could facilitate direct, unbiased identification of both genomic sequence and the corresponding proteome at these sites of open chromatin. The present invention provides a dual transposase/peroxidase approach, which we call integrative DNA And Protein Tagging (iDAPT), to tag and enrich both DNA sequence (iDAPT-seq) and protein content (iDAPT-MS) associated with regions of open chromatin, attainable from a single nuclear preparation. This technology captures genomic profiles of open chromatin, while facilitating the discovery of additional open chromatin protein markers, including, e.g., CCDCl2 and SNRPA. iDAPT expands the repertoire of active sequence-specific transcription factors detectable by sequencing-based modalities and enables the inference of gene regulatory networks and transcription factor complexes. To demonstrate the power of this dual tagging approach, we applied iDAPT to profile changes to the epigenomic landscape induced by mutant isocitrate dehydrogenase 2 (mIDH2) in acute myeloid leukemia (AML), driven in part by the neomorphic production of the oncometabolite (R)-2-hydroxyglutarate (R-2HG) (Dang et al., Nature 462:739-744, 2009; Mardis et al., N. Engl. J. Med. 361:1058-1066, 2009; Losman et al., Science 339:1621-1625, 2013; Kats et al., Cell Stem Cell 14:329-341, 2014; Quek et al., Nat. Med. 24:1167-1177, 2018). Integration of iDAPT-MS and iDAPT-seq implicates the dissociation of TAL1 from the GATA1 pioneer transcription factor at the core of the block of terminal erythroid differentiation in mIDH2 AML. Our findings demonstrate the power of iDAPT as a discovery platform for both the dynamic epigenomic landscapes and their active transcription factor components associated with biological phenomena and disease.

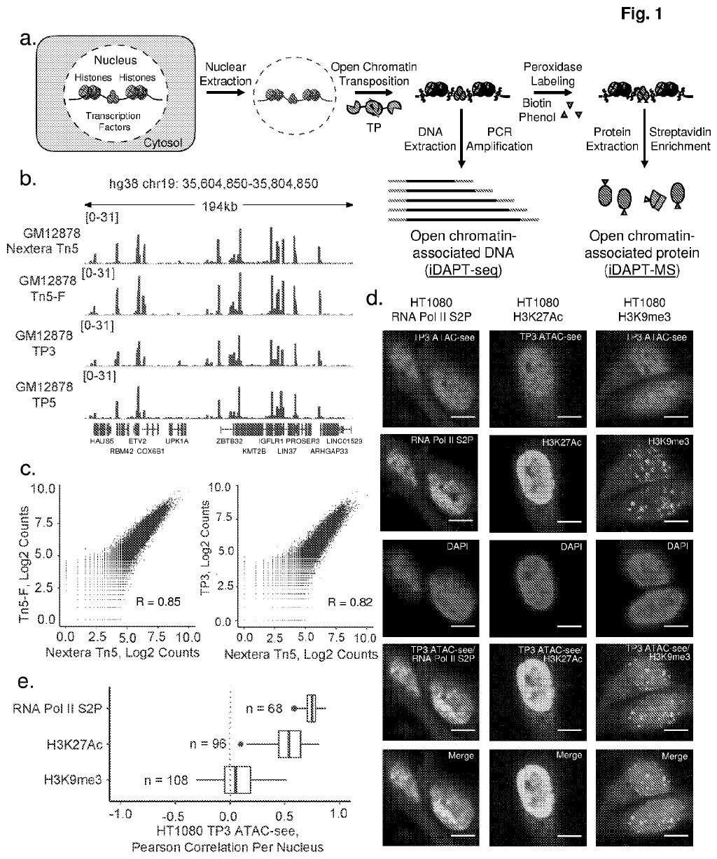

We thus developed the iDAPT platform to profile the genomic and proteomic components of open chromatin from a single lysate via a recombinant bifunctional transposase/peroxidase probe (FIG. 1a). We used the Tn5 transposase for this purpose, which tags and fragments (tagments) DNA and remains physically bound to its DNA substrate after insertion of its transposon payload (Reznikoff, Annu. Rev. Genet. 42:269-286, 2008). Because Tn5 transposase preferentially tagments sterically accessible DNA in native chromatin, we considered that Tn5 transposase may also serve as a biochemical anchor to facilitate proximal labeling of proteins associated with open chromatin (FIG. 1a). The APEX2 peroxidase was selected for use due to, e.g., its short labeling timeframe of one minute and its peroxidase activity as a purified protein (Lam et al., Nat. Methods 12:51-54, 2014; Paek et al., Cell 169:338-349.e11, 2017). Accordingly, we fused APEX2 with Tn5 transposase for concomitant transposition and peroxidase-mediated biotin labeling.

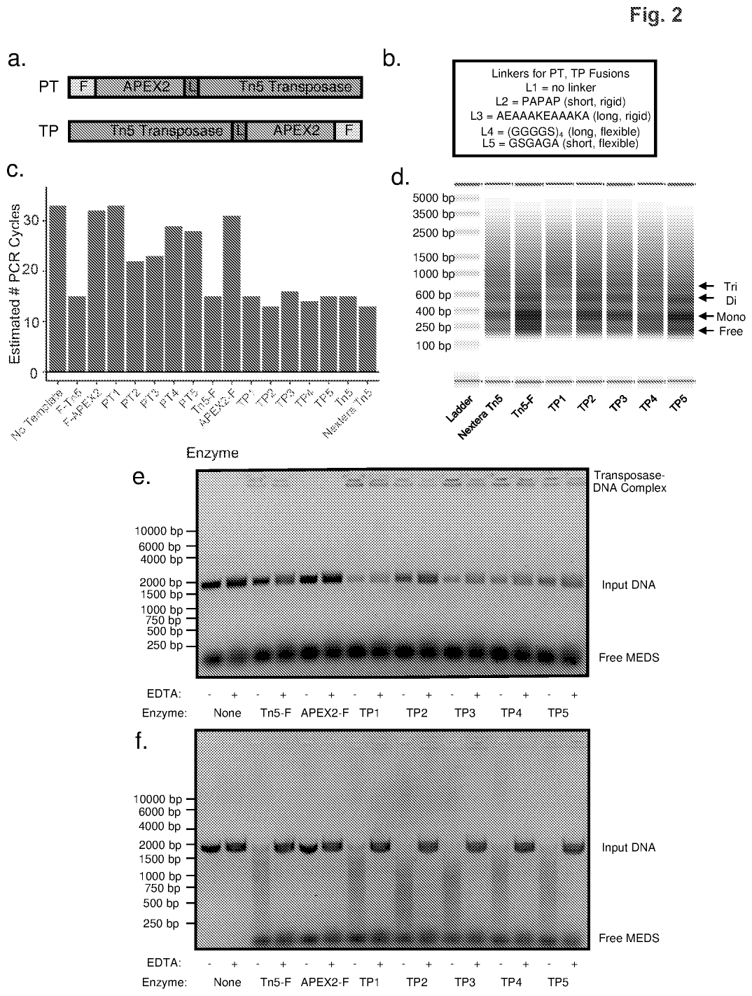

We cloned and purified recombinant APEX2 peroxidase fused both N- and C-terminally to Tn5 transposase (peroxidase/transposase or PT; transposase/peroxidase or TP) adjoined via several linkers (L1-L5) to identify a fusion enzyme with robust ability to label both DNA and protein associated with open chromatin (FIG. 2a-b). We first tested our fusion enzymes for transposase domain activity via qPCR quantification of pre-amplified ATAC-seq libraries generated from GM12878 cells. N-terminal transposase (TP1-TP5) fusions yielded sequencing library abundances similar to Tn5 transposase alone, whereas C-terminal transposase (PT1-PT5) fusions broadly exhibited decreased transposase activity (FIG. 2c). DNA fragment size analysis of ATAC-seq libraries generated from all TP fusions yielded a fragment size distribution corresponding to ˜200 base pair-wide nucleosomal periods typically observed with open chromatin enrichment (Buenrostro et al., Nat. Methods 10:1213-1218, 2013) (FIG. 2d), suggesting that the peroxidase domain abutting Tn5 transposase in our TP fusion probes does not broadly affect transposase activity. In agreement with previous reports of stable Tn5 transposase-DNA complex formation after tagmentation (Reznikoff, Annu. Rev. Genet. 42:269-286, 2008), we observed a gel shift of linearized plasmid in the presence of transposase domain-containing enzymes but not in the presence of the APEX2 domain alone, with corresponding DNA fragmentation profiles dependent on both transposase-DNA association and absence of the divalent cation chelator EDTA (Chen et al., Nat. Methods 13:1013-1020, 2016; Buenrostro et al., Nature 523:486-490, 2015) (FIG. 2e-f).

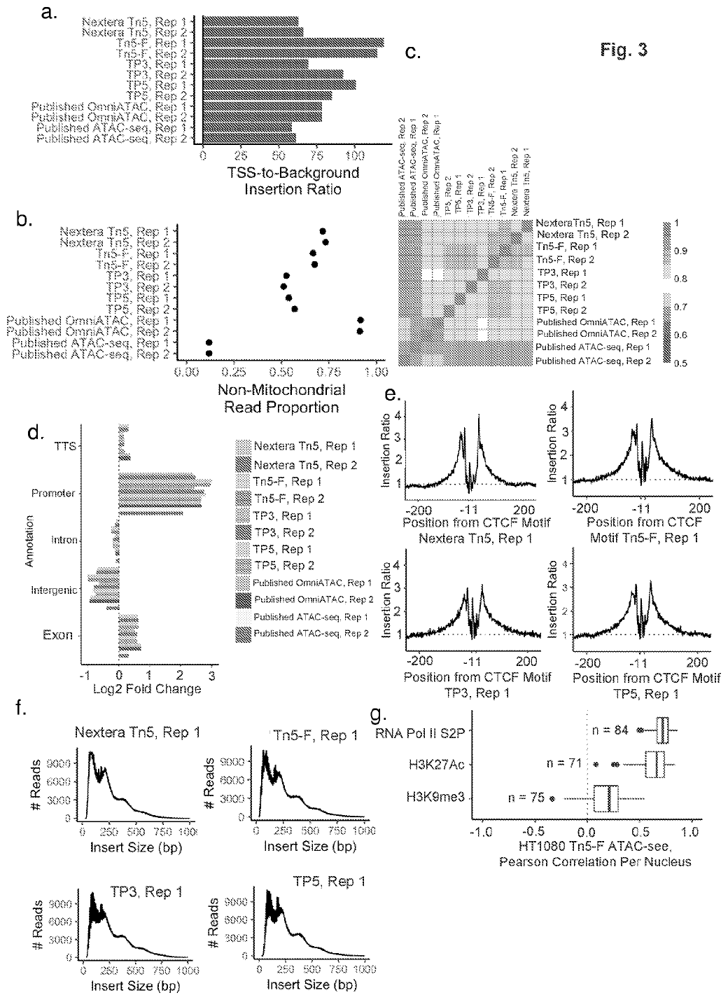

To ensure further that transposase preference for open chromatin is not altered by the C-terminal APEX2 peroxidase, we generated ATAC-seq/iDAPT-seq libraries of GM12878 cells with the fusion probes TP3 and TP5 and subjected them to next generation sequencing using a recently optimized ATAC-seq protocol (Corces et al., Nat. Methods 14:959-962, 2017). Distinct from current transposase-based accessibility profiles such as ATAC-seq, iDAPT-seq uses TP fusion enzymes for tagmentation, allowing for concomitant heme-based peroxidation for proteome labeling (FIG. 1a). iDAPT-seq libraries from TP3 and TP5 exhibited high signal-to-noise ratios, akin to ATAC-seq libraries from Nextera Tn5 transposase alone or FLAG-tagged Tn5 transposase (Tn5-F; purified in-house) (FIG. 1b, FIG. 3a). We observed a similar proportion of reads aligning to the mitochondria genome independent of enzyme, in line with known mitochondrial enrichment (Buenrostro et al., Nat. Methods 10:1213-1218, 2013; Corces et al., Nat. Methods 14:959-962, 2017) (FIG. 3b). Correlation analyses confirmed no substantial differences in transposase insertion preferences across the open chromatin landscape, despite the presence of the peroxidase domain (FIG. 1c, FIG. 3c). In addition, enriched genic features, insertion preferences, and fragment size distributions are all similar, with no significant differences (FIG. 3d-f). To further confirm that TP fusions behave as Tn5 transposase alone, we performed ATAC-see in HT1080 cells (Chen et al., Nat. Methods 13:1013-1020, 2016). Tagmentation activity via the TP3 probe was found to mimic Tn5 transposase activity, strongly correlating with histone H3 lysine 27 acetylation (H3K27Ac) and RNA Polymerase II serine 2 phosphorylation (RNAPII S2P) immunofluorescence signal, markers of transcriptionally active chromatin, and poorly correlating with H3K9me3, a marker of transcriptionally inactive chromatin (FIG. 1d-e, FIG. 3g). Taken together, these data indicate that our TP fusion probes retain native Tn5 transposase activity and preferentially tag genomic regions of open chromatin.

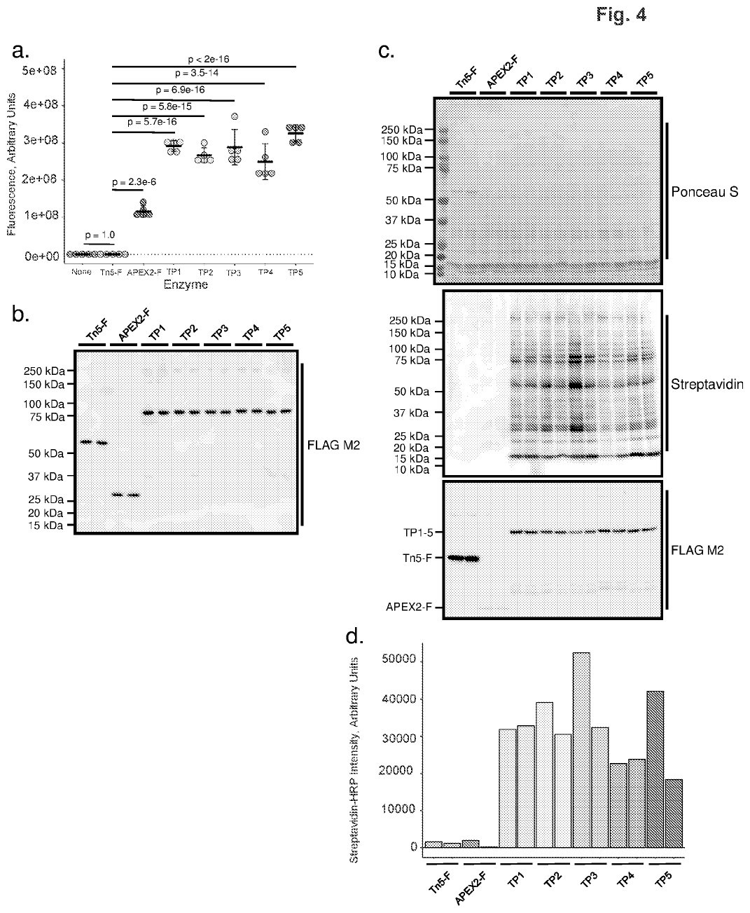

Having confirmed TP fusion tagging of and localization to open chromatin, we assessed recombinant APEX2 peroxidase functionality when fused with Tn5 transposase. Peroxidase activity was detected via resorufin fluorescence in the presence of an APEX2 peroxidase domain-only enzyme and all fusion proteins except for a Tn5 transposase domain-only enzyme, confirming peroxidase-dependent enzymatic activity (FIG. 4a). To determine the potential for proteomic labeling with our purified TP fusion enzymes, we performed peroxidase-mediated biotin labeling in GM12878 nuclei after transposition of native chromatin, using the anchoring of the transposase domain to its DNA substrate for targeted proximity labeling. Transposase domain-containing enzymes are detectable in labeled nuclei by western blotting, whereas APEX2-only enzyme is nearly undetectable after washing and peroxidase-mediated biotin labeling (FIG. 4b-c). Accordingly, robust biotinylation is observed only when both enzymatic domains are present on the biochemical probe, with the highest signals arising from the TP3 and TP5 fusion proteins (FIG. 4b-d). Our findings validate the requirement for both transposase and peroxidase enzymatic domains to label proteins with biotin in nuclear extracts.

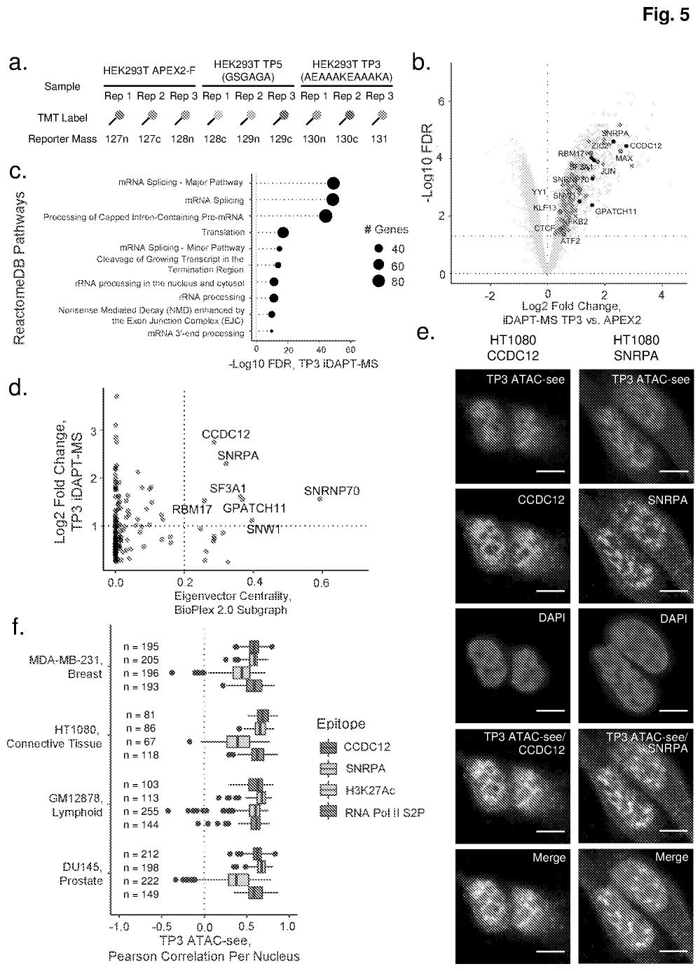

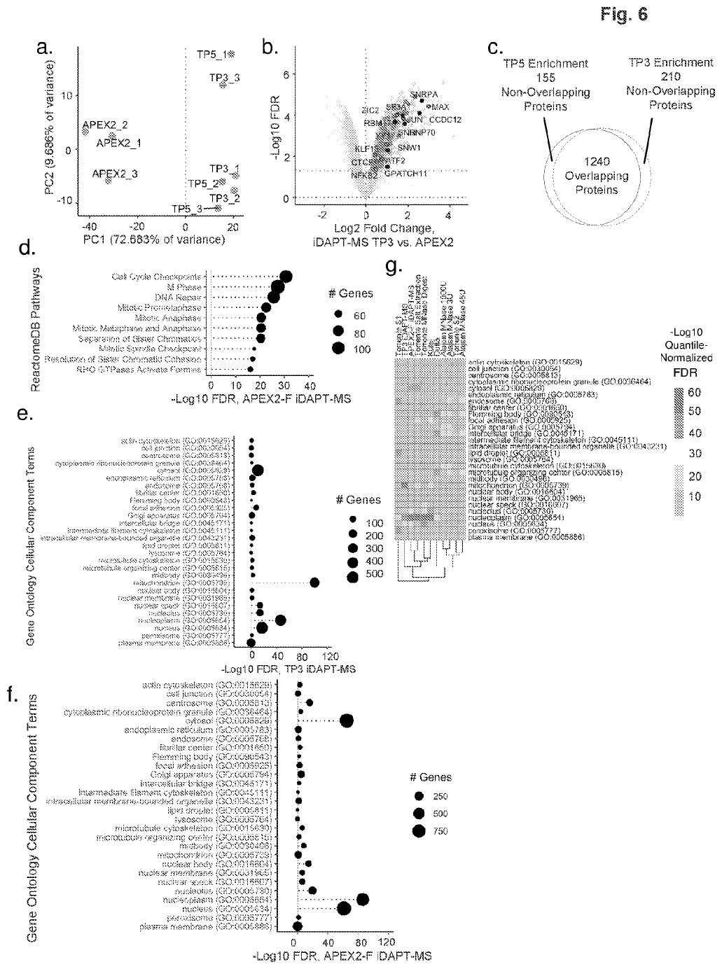

With the components of iDAPT in hand, we characterized the extent of open chromatin proteomic enrichment by quantitative mass spectrometry (iDAPT-MS). We compared proteomic labeling and quantitative enrichment via transposase-directed APEX2 labeling with TP3 and TP5 versus enrichment with free APEX2 alone in HEK293T nuclei, using streamlined tandem mass tagging (SL-TMT)(Navarrete-Perea et al., J. Proteome Res. 17:2226-2236, 2018) of peptides for sample multiplexing and synchronous precursor selection mass spectrometry (SPS-MS3)(Ting et al., Nat. Methods 8:937-940, 2011; McAlister et al., Anal. Chem. 86:7150-7158, 2014) for downstream peptide identification and quantitation (FIG. 5a). With iDAPT-MS, we identified a total of 20,184 peptides and 6,245 proteins across nine TMT channels (FIG. 5b). We observed a similar separation of both TP3 and TP5 from APEX2 enrichment along the first principal component, confirming a similar degree of specificity between the two probes (FIG. 6a). Of significant proteins enriched by TP3 and TP5 at an FDR threshold of 5%, the vast majority of proteins identified were shared between both fusions (1,240 proteins) (FIG. 6b). Reflective of our previous observation of increased biotin labeling of TP3 over TP5, we found that TP3 enriches for slightly more proteins (1,450) than TP5 (1,395) (FIG. 5b, FIG. 6b-c). Numerous sequence-specific transcription factors such as MAX and JUN are detectable in the TP3- and TP5-enriched proteomes (FIG. 5c). Additionally, TP3 labels RNA processing and splicing components among ReactomeDB pathways (Fabregat et al., Nucleic Acids Res. 46:D649-D655, 2018), whereas APEX2 alone labels components associated with mitosis (FIG. 5c, FIG. 6d). We detected enrichment of both nuclear and mitochondrial proteins from subcellular enrichment analysis of TP3-labeled nuclear proteomes; on the other hand, mitochondrial enrichment is substantially lost among non-fusion APEX2-labeled proteins, validating the preferential labeling of proteins in the vicinity of known Tn5 transposase localization to the nucleus and mitochondria (Buenrostro et al., Nat. Methods 10:1213-1218, 2013; Corces et al., Nat. Methods 14:959-962, 2017) (FIG. 6e-f). Furthermore, iDAPT-MS yields similar or increased enrichment of nuclear proteins over non-nuclear proteins when compared to other biochemical enrichment methods for open chromatin-associated proteins (Torrente et al., PLoS One 6:e24747, 2011; Alajem et al., Cell Rep. 10:2019-2031, 2015; Dutta et al., Mol. Cell. Proteomics 13:2183-2197, 2014; Kulej et al., Mol. Cell. Proteomics 16:S92-S107, 2017) (FIG. 6g). These results confirm the ability of iDAPT-MS to elucidate the transposase-accessible proteome.

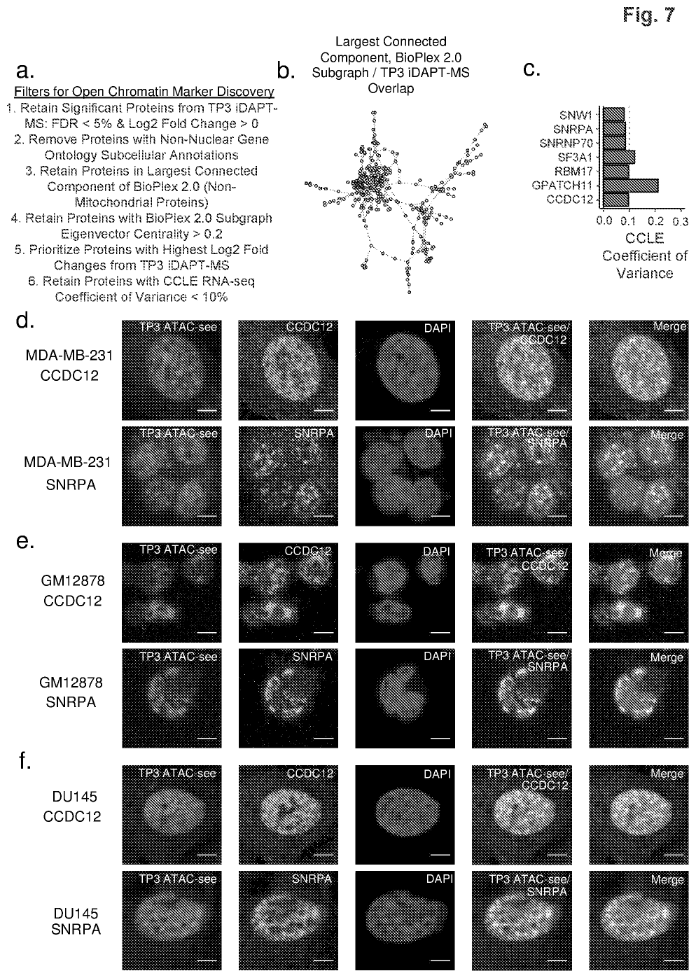

As TP3 tagmentation activity positively correlates with known markers of open chromatin including H3K27Ac and RNAPII S2P, we evaluated iDAPT-MS for its ability to identify additional protein markers associated with open chromatin. Starting from our set of significantly enriched proteins from iDAPT-MS, we excluded proteins with annotated Gene Ontology subcellular localization outside of the nucleus (The Gene Ontology Consortium, Nucleic Acids Res. 47:D330-D338, 2019) (FIG. 7a). We also posited that putative biomarkers should exhibit broad connectivity within the open chromatin-enriched proteome. To do this we integrated the set of non-mitochondrial proteins enriched via iDAPT-MS with protein-protein interaction information from the BioPlex 2.0 network (Huttlin et al., Nature 545:505-509, 2017) and filtered by eigenvector centrality (FIG. 5d, FIG. 7b). Finally, we removed proteins with a high coefficient of variance (>10%) in gene expression across the ˜1,000 cancer cell lines from the Cancer Cell Line Encyclopedia (Ghandi et al., Nature doi:10.1038/s41586-019-1186-3, 2019) (FIG. 7c). We identified CCDCl2 and SNRPA, the most enriched proteins from iDAPT-MS that also passed our filtering strategy, in addition to proteins associated with splicing (FIG. 5d). We confirmed by co-immunofluorescence staining with TP3 ATAC-see that CCDCl2 and SNRPA colocalize with open chromatin to a similar degree as the euchromatin markers H3K27Ac and RNAPII S2P in multiple cell lines (FIG. 5e-f, FIG. 7d-f). In this manner, iDAPT-MS facilitates the identification of novel protein associations with open chromatin and points to components of the spliceosome machinery as an integral component of open chromatin architecture.

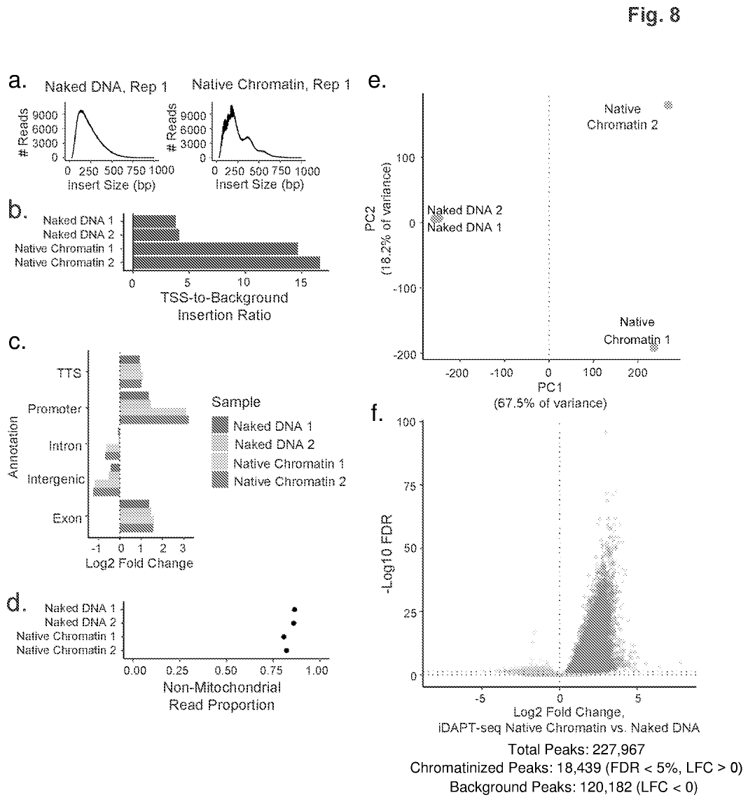

Through integration of both iDAPT-MS and iDAPT-seq, we hypothesized that our approach may enable identification of the sequence-specific transcription factors active in transcriptional regulation in the cell. To determine the degree of concordance between genomic and proteomic enrichment of sequence-specific transcription factors by iDAPT, we carried out iDAPT-seq analysis with both HEK293T cells and their “naked” genomic DNA. Insertion size analysis reveals nucleosomal positioning in native chromatin that is lost in naked DNA (FIG. 8a). This chromatin architecture is also apparent in the native chromatin setting by the relative increase in transposon insertions at transcription start sites and promoter regions and a decrease in insertions within intronic and intergenic regions across the genome (FIG. 8b-c). In line with our observed mitochondrial enrichment by iDAPT-MS, a proportion of sequencing reads (˜15-20%) maps to the mitochondrial genome, with a slightly increased proportion from native chromatin (FIG. 6e, 8d). Across peaks of transposition enrichment, iDAPT-seq profiles of native chromatin and naked DNA segregate along the first principal component (FIG. 8e); furthermore, peaks enriched in native chromatin broadly exhibit stronger statistical significance as compared with peaks enriched in naked DNA (FIG. 8f). These findings led us to conclude that iDAPT-seq reveals a pattern of well-positioned regions of chromatin accessibility, largely at gene regulatory regions, that is dependent on native chromatin architecture.

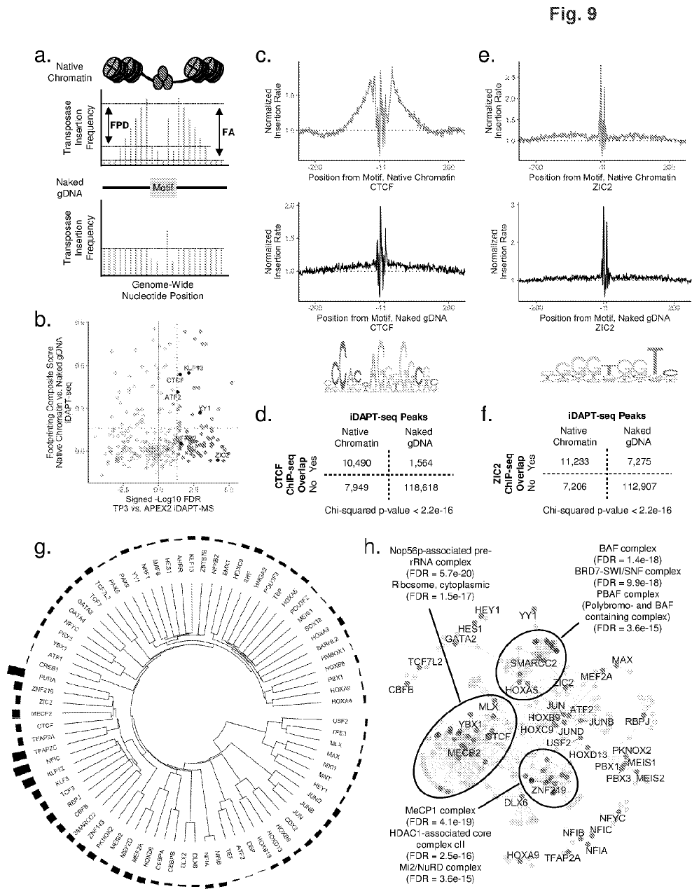

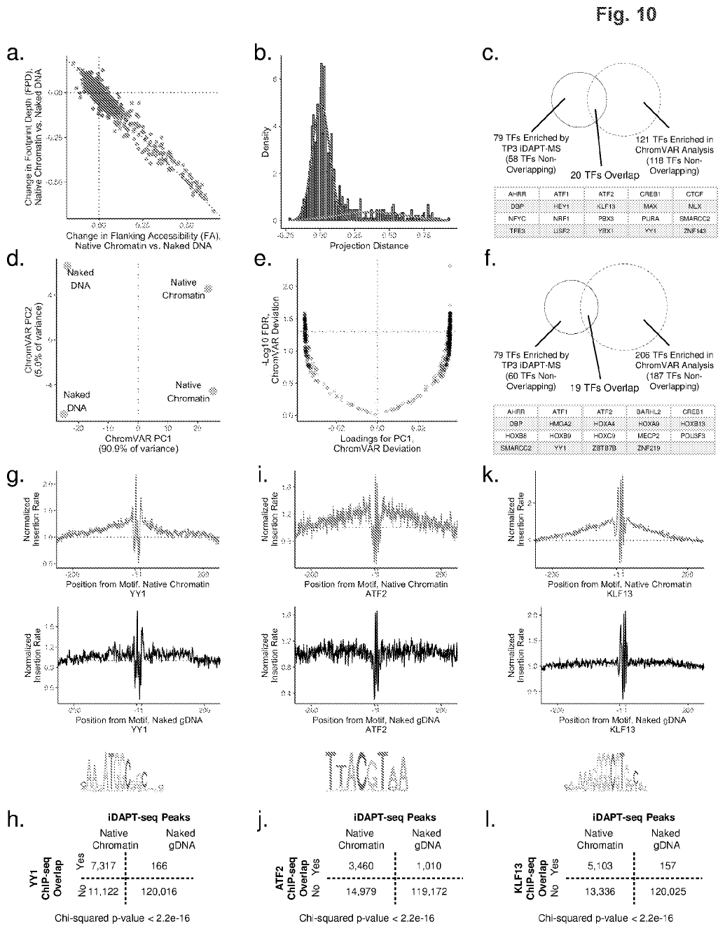

We next determined the repertoire of sequence-specific transcription factors from CisBP (Weirauch et al., Cell 158:1431-1443, 2014) enriched on open chromatin using both a bivariate footprinting approach (Corces et al., Science 362 (6413), 2018; Baek et al., Cell Rep. 19:1710-1722, 2017), accounting for both the depth of a transcription factor footprint and flanking chromatin accessibility about the transcription factor motif, and a motif enrichment approach via ChromVAR (Schep et al., Nat. Methods 14:975-978, 2017) (FIG. 9a, FIG. 10a-f). After filtering by detectable gene expression in HEK293T cells from published mRNA-seq datasets, we identified 139 transcription factors enriched by bivariate footprinting analysis and 206 transcription factors enriched by ChromVAR (FIG. 10c, f). Of the 79 CisBP transcription factors significantly enriched by TP3 from iDAPT-MS, 21 and 19 transcription factors are concordant with bivariate footprinting and ChromVAR analyses of iDAPT-seq profiles, respectively, with 7 transcription factors being concordant by all three methods (FIG. 9b, FIG. 10c, f). CTCF, an insulator protein with a long retention time on DNA (Nakahashi et al., Cell Rep. 3:1678-1689, 2013), exhibits a strong footprint and is detected by both iDAPT-MS and ChIP-seq (FIG. 9c-d). Other transcription factors with detectable footprints are also detected by both iDAPT-MS and ChIP-seq (FIG. 10g-1). Accordingly, transcription factors identified by both iDAPT-seq and iDAPT-MS enrichment analyses represent high-confidence transcription factors for a particular cellular state. At the same time, our analysis also highlights transcription factors that are clearly enriched by iDAPT-MS, yet exhibit weak footprinting profiles, including NFKB2 and ZIC2-NF-κB complexes, which have short DNA residence times and thus weak footprinting potential (Bosisio et al., EMBO J. 25:798-810, 2006), and ZIC2 ChIP-seq peaks are enriched across open chromatin (FIG. 9b, e-f). Thus, iDAPT-MS and iDAPT-seq together capture an expanded compendium of transcription factors associated with transcriptional regulation in the cell.

Using the set of 79 significant iDAPT-MS transcription factors, we sought to identify associations between the various transcription factors as detectable via iDAPT-seq and iDAPT-MS. We matched iDAPT-seq peaks with transcription factor motifs to infer binding positions of each transcription factor across the open chromatin landscape (FIG. 9g). Hierarchical clustering broadly reveals clustering of transcription factor families, likely a consequence of consensus motif similarity. For instance, MNT, MXI1, MAX, MLX, TFE3, USF2, and HEY1 all share a 5′-CACGTG-3′ consensus motif annotated by CisBP. Accordingly, these seven transcription factors cluster closely with each other. This clustering similarity may be a consequence of transcriptional cooperativity, as MAX, MNT, MXI1, and MLX form transcription factor heterodimers with each other (Conacci-Sorrell et al., Cold Spring Harb. Perspect. Med. 4:a014357, 2014), or possible competition for these motif regions. In parallel, we assembled a transcription factor complex network using these transcription factors and collating their first order protein interactors from the BioPlex network with overlap of our iDAPT-MS data (FIG. 9h). We observed a large connected component encompassing many transcription factors, including CTCF, SMARCC2, and the JUN/JUNB/JUND transcription factor complex, and smaller subgraphs associated with lower vertex count. Within the largest connected component, we identified enrichment of ribosome, chromatin remodeling, and histone deacetylase CORUM complexes (Ruepp et al., Nucleic Acids Res. 36:D646-50, 2008), suggestive of coordination between these different components on open chromatin through these sequence-specific transcription factors (FIG. 9h). Both iDAPT-MS and iDAPT-seq are able to capture interactions between various transcription factor components, facilitating the inference of cis-regulatory transcription factor networks and their corresponding protein interactors with increased confidence.

To demonstrate the power and versatility of our iDAPT approach to inform the dynamic nature of open chromatin, we next examined the changes to the epigenomic landscape induced by mutations in the IDH2 enzyme in AML. Recurrent point mutations in the isocitrate dehydrogenase enzymes IDH1 and IDH2 are observed in 10-20% of patients with AML as well as gliomas and other cancers, directly linking aberrations in cellular metabolism with dysregulation of chromatin architecture through production of R-2HG from its canonical metabolic product, 2-oxoglutarate (2OG) (Dang et al., Nature 462:739-744, 2009; Mardis et al., N. Engl. J. Med. 361:1058-1066, 2009; Losman et al., Science 339:1621-1625, 2013; Losman et al., Genes Dev. 27:836-852, 2013). R-2HG inhibits numerous 2OG-dependent enzymes, including the JmjC histone lysine demethylase (KDM) and TET 5-methylcytosine DNA hydroxylase epigenetic modifier families, to promote neoplastic transformation and a block in differentiation (Losman et al., Science 339:1621-1625, 2013; Kats et al., Cell Stem Cell 14:329-341, 2014; Quek et al., Nat. Med. 24:1167-1177, 2018). While the proto-oncogenic consequences associated with mutant IDH1/2 status and R-2HG production in AML are well-defined, including erythroid differentiation blockade (Losman et al., Science 339:1621-1625, 2013; Kats et al., Cell Stem Cell 14:329-341, 2014; Quek et al., Nat. Med. 24:1167-1177, 2018), the specific epigenetic mechanisms underpinning their ability to enhance leukemic progression largely remain uncharacterized. More urgently, the emergence of resistance to targeted therapies against mutant IDH1/2 enzymes suggests a critical need to understand the downstream consequences of R-2HG perturbation (Quek et al., Nat. Med. 24:1167-1177, 2018; Intlekofer et al., Nature 559:125-129, 2018; Harding et al., Cancer Discov. 8:1540-1547, 2018).

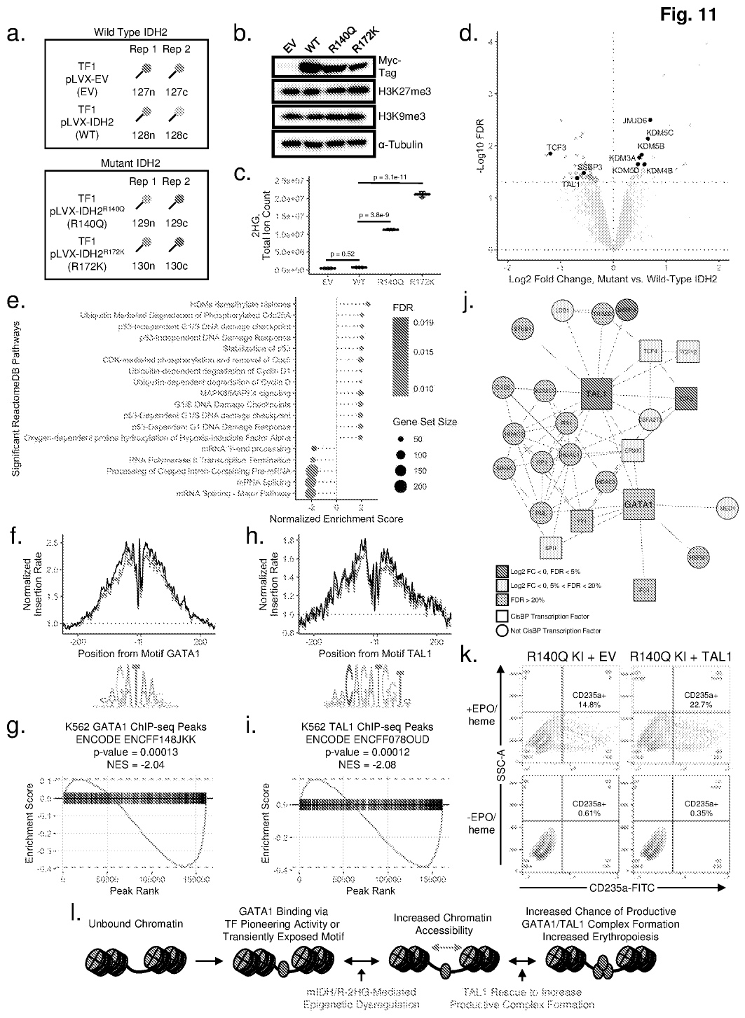

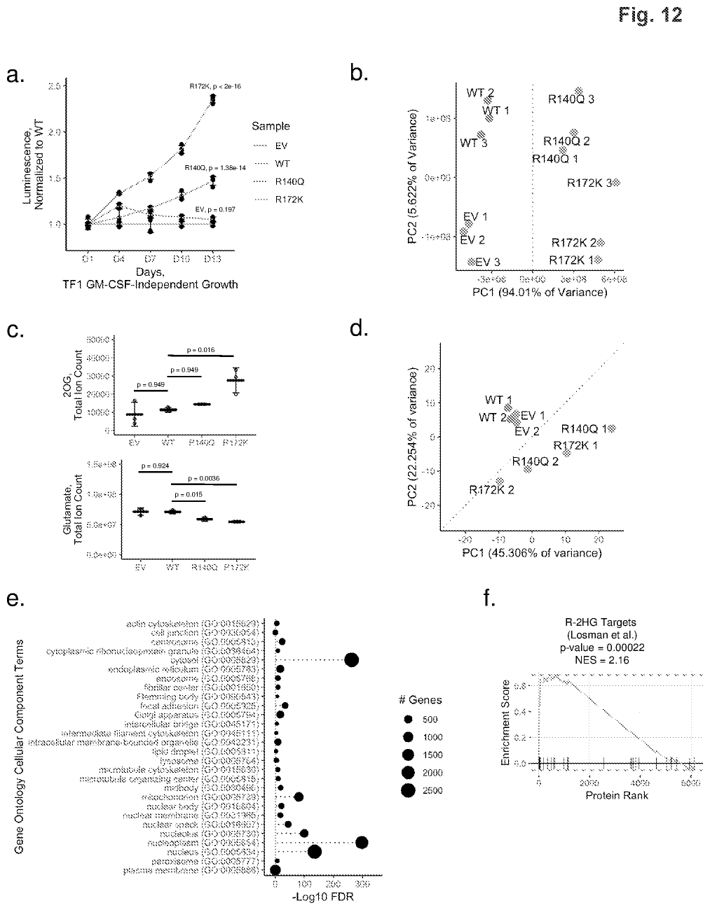

To elucidate the epigenomic landscape induced by mIDH2, we used a well-characterized cancer cell line model of mIDH2 AML, comprising of the TF1 erythroleukemia cell line transduced with the R140Q or R172K point mutants of IDH2 or wild-type controls (Losman et al., Science 339:1621-1625, 2013) (FIG. 11a-b). TF1 cells transduced with mIDH2 constructs exhibit increased histone methylation, R-2HG metabolite levels determined by 2HG total ion counts from mass spectrometry, and cytokine-independent proliferation relative to cells transduced with wild-type constructs (FIG. 11b-c, FIG. 12a). Metabolite profiling of these cells reveals a clear separation between mutant and wild type IDH2-transduced cells along the first principal component—in addition to increased R-2HG levels, our mIDH2 cells are marked by decreased glutamate levels and a nonsignificant increase in 2OG levels (FIG. 12b-c). These results confirmed that our cells are representative of previously reported mIDH1/2-associated molecular phenotypes (Losman et al., Science 339:1621-1625, 2013; Losman et al., Genes Dev. 27:836-852, 2013; Mugoni et al., Cell Res. doi:10.1038/s41422-019-0162-7, 2019). We next performed iDAPT on these cells, with each sample processed in duplicate (FIG. 11a). From iDAPT-MS analysis, we identified 33,040 peptides and 6,479 proteins, with proteomic profiles linearly separating by IDH2 mutant status via principal component analysis (FIG. 11d, FIG. 12d). Proteins detected by iDAPT-MS are predominantly enriched for nuclear, cytosolic, and mitochondrial localization patterns and include both CCDCl2 and SNRPA, in line with our findings above (FIG. 12e). We surprisingly observed multiple JmjC-class histone lysine demethylases (e.g., JMJD6, KDM4B, and KDMSC), which use 2OG as a cofactor and are inhibited by R-2HG, to be significantly enriched on open chromatin in the mutant IDH2 setting, a pattern corroborated by gene set enrichment analysis using ReactomeDB pathway annotations as well as previously reported enzymatic targets of R-2HG (Losman et al., Genes Dev. 27:836-852, 2013) (FIG. 11d-e, FIG. 12f). Additional significantly enriched ReactomeDB pathways include DNA repair, consistent with double-stranded DNA repair dysfunction as a consequence of KDM4A/B inhibition by R-2HG (Sulkowski et al., Sci. Transl. Med. 9 (375), 2017; Inoue et al., Cancer Cell 30:337-348, 2016), and mRNA splicing, recently implicated in mIDH1/2 pathophysiology due to somatic mutations in splicing components arising as a consequence of resistance to mutant IDH2-targeted therapy (Quek et al., Nat. Med. 24:1167-1177, 2018) (FIG. 11e). Thus, iDAPT-MS, as applied to our model of mIDH in the TF1 cell line, not only corroborates previously reported mechanistic associations with mIDH status, but also highlights previously unappreciated epigenetic consequences of this genetic perturbation.

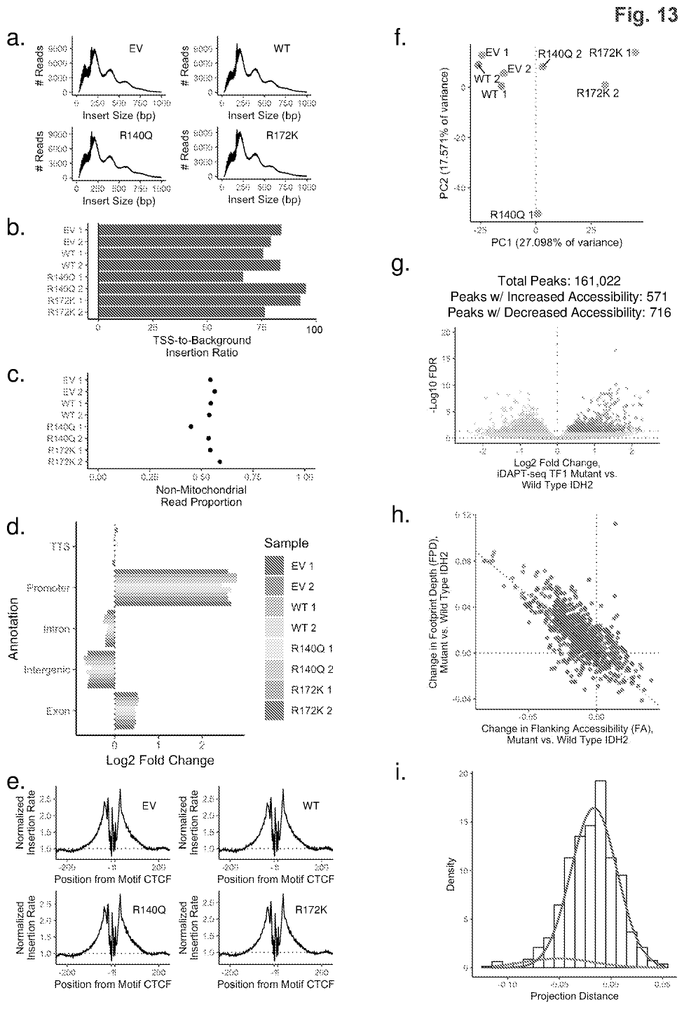

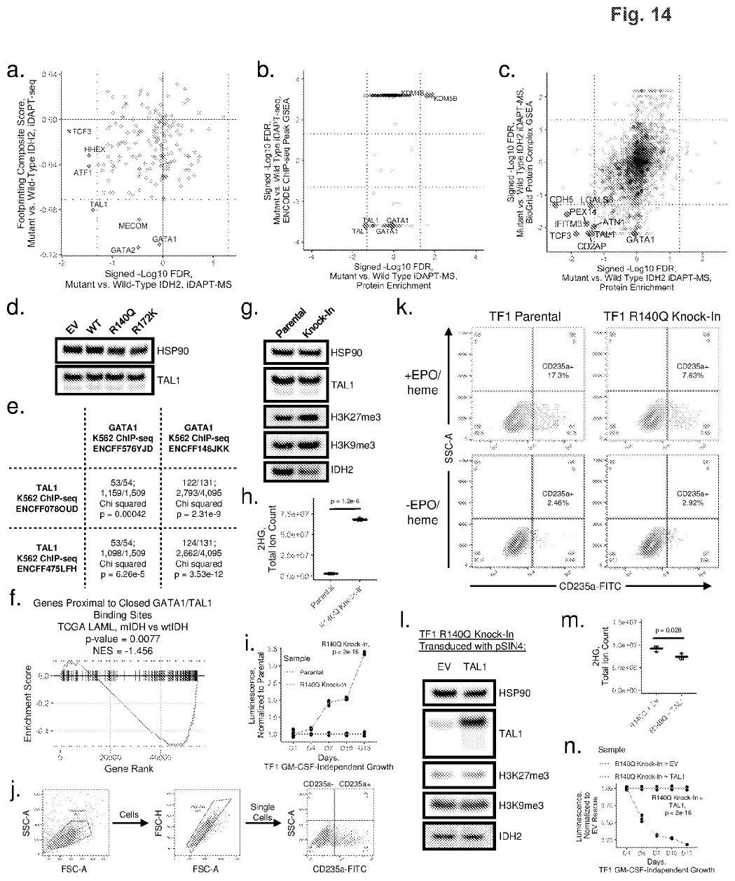

As excess production of R-2HG leads to abrogated erythropoiesis in AML (Losman et al., Science 339:1621-1625, 2013; Kats et al., Cell Stem Cell 14:329-341, 2014; Quek et al., Nat. Med. 24:1167-1177, 2018), we assessed for detectable changes in chromatin accessibility patterns via iDAPT-seq. We did not observe any overt biological differences between wild type and mutant IDH2 contexts by insert size distribution, genic enrichment, mitochondrial contamination, or insertion preference (FIG. 13a-e). On the other hand, chromatin accessibility profiles at the level of peaks separated by mutation along the first principal component, was suggestive of chromatin context-specific epigenetic changes (FIG. 13f). Of 161,022 total peaks, 571 and 716 peaks are associated with significantly increased and decreased accessibility, respectively, as a consequence of mIDH2 perturbation (FIG. 13g). Bivariate footprinting and K562 erythroleukemia ChIP-seq enrichment analyses of our iDAPT-seq data implicate mIDH2-induced perturbations of transcription factor activity of GATA1, previously inferred to be dysregulated in mIDH1/2 AML (Kats et al., Cell Stem Cell 14:329-341, 2014; Figueroa et al., Cancer Cell 18:553-567, 2010), and TAL1 (FIG. 11f-i, FIG. 13h-l, 14a-b). GATA1 and TAL1 are master regulators of erythroid differentiation that together form a protein complex (Porcher et al., Blood 129:2051-2060, 2017), and loss of these erythroid transcription factors in the mIDH1/2 setting may explain the observed block in terminal erythroid differentiation. Unexpectedly, while both GATA1—(EP300, MED1, SPI1) and TAL1-centric (SSBP3, TCF3, TCF4, TCF12, CBFA2T3, EP300, LDB1) protein complex components also exhibit decreased association with open chromatin in the mIDH2 context, GATA1 protein itself is detected but not significantly perturbed by mIDH2 status as measured by iDAPT-MS, despite concordance with TAL1 loss (FIG. 11j, FIG. 14c). This discordance may be explained by the transcription factor pioneering activity of GATA1, binding to DNA independent of chromatin accessibility status (Kadauke et al., Cell 150:725-737, 2012). While GATA1 binding to DNA leads to increased proximal chromatin accessibility to unveil nearby TAL1 binding motifs (Hu et al., Genome Res. 21:1650-1658, 2011; Wu et al., Genome Res. 24:1945-1962, 2014; Wakabayashi et al., Proc. Natl. Acad. Sci. U.S.A. 113:4434-4439, 2016), GATA1-mediated chromatin remodeling activity may be diminished due to proximal dysregulated DNA and histone methylation states induced by R-2HG (Dann et al., Nature 548:607-611, 2017), thereby attenuating TAL1 localization and concomitant erythroid differentiation. Accordingly, we observed no significant changes in TAL1 global protein levels across our TF1 cell lines, ruling out changes in steady state levels of TAL1 protein (FIG. 14d). Among peaks with significantly decreased chromatin accessibility in the mIDH2 setting, almost every overlapping GATA1 ChIP-seq peak also contains a TAL1 ChIP-seq peak, whereas among peaks with increased chromatin accessibility, GATA1 ChIP-seq peaks contain fewer TAL1 ChIP-seq peaks (93-98% vs. 65-77% of GATA1 peaks contain TAL1 peaks; FIG. 14e). Furthermore, we found that the expression levels of genes proximal to inaccessible TAL1/GATA1 sites are negatively enriched in transcriptome profiles from AML samples with mutations in IDH1/2 versus those with wild type IDH1/2 across the TCGA AML patient cohort (Cancer Genome Atlas Research Network et al., N. Engl. J. Med. 368:2059-2074, 2013) (FIG. 14f). Taken together, iDAPT-seq and iDAPT-MS point to TAL1 loss of function as a consequence of mIDH1/2 genetic perturbation, prohibiting remodeling of chromatin proximal to a subset of GATA1-bound genetic loci to effect erythroid differentiation.

Finally, we assessed whether increased TAL1 expression may rescue attenuation of erythroid differentiation in the mIDH2 context. We hypothesized that increased steady state levels of TAL1 may overcome mIDH2-induced chromatin inaccessibility at GATA1-bound loci by increasing the likelihood of formation of productive GATA1/TAL1 complexes to promote erythroid differentiation. We confirmed increased histone methylation, increased R-2HG levels, increased cytokine-independent proliferation, and decreased sensitivity to erythropoietin (EPO)/heme-induced erythroid differentiation of TF1 cell lines with an IDH2 R140Q knock-in mutation relative to parental TF1 cells (FIG. 14g-k). In this mIDH2 knock-in cell line, we transduced lentiviral constructs either containing the TAU open reading frame or empty vector. While TAL1 lentiviral rescue did not substantially affect R-2HG levels nor histone methylation (FIG. 14l-m), TAL1 both attenuated the cytokine-independent growth and sensitized cellular response to EPO/heme-mediated differentiation as compared to transduction with empty vector alone (FIG. 11k, FIG. 14n). These data reify functional loss of TAL1 in aberrant erythropoiesis as a downstream consequence of epigenomic rewiring induced by mutant IDH1/2, which may be rescued by increased TAL1 expression and transcription factor activity.

In summary, we report the first application of a dual transposase/peroxidase tagging approach to obtain a systemic overview of the epigenomic landscape. Our iDAPT platform is able to identify genomic regulatory positions, sequence-specific transcription factors with long and short retention times on DNA, and additional associated proteins across accessible chromatin. Further, we may infer transcription factor gene targets and their protein complex components to obtain a complete portrait of cis-regulation within the cell. As iDAPT does not require genetic manipulation of biological samples of interest, our approach may be readily applied to numerous biological phenomena, including patient samples, to uncover molecular pathologies underpinning a given disease state. Application of iDAPT to elucidate the epigenomic changes in response to IDH2 point mutations in AML unveils changes in both proteome composition and genomic accessibility due to perturbation by the neomorphic metabolic product R-2HG. Through integration of iDAPT-MS and iDAPT-seq, we identified a loss of TAL1, a critical regulator of normal erythropoiesis, from open chromatin as a consequence of mIDH2 perturbation. We propose a mechanistic model of mIDH1/2-induced erythropoietic dysfunction, whereby TAL1 association with GATA1 bound on regions of open chromatin is attenuated, leading to decreased cis-regulation of gene expression, a block in erythroid differentiation, and ultimately erythroid/myeloid hematopoietic skewing as observed in AML patients with these mutant alleles (Quek et al., Nat. Med. 24:1167-1177, 2018) (FIG. 11l). Importantly, TAL1 rescues cytokine dependence and sensitizes cells to EPO/heme-mediated differentiation in a knock-in of the IDH2^{R140Q} mutation in the TF1 cell line, suggesting a potential therapeutic node for patients with mIDH1/2-driven AML. Our data substantiate the power of iDAPT to unravel epigenomic landscapes underpinning normal development and disease states in both model systems and patient-derived samples.

### Methods

No statistical methods were used to predetermine sample size. The experiments were not randomized. The investigators were not blinded to allocation during experiments and outcome assessment.

Cell lines and culture conditions. GM12878 cells (Coriell) were cultured in RPMI-1640 supplemented with L-glutamine (Gibco) supplemented with 15% fetal bovine serum (FBS) and 1% penicillin/streptomycin (Thermo Fisher Scientific). HT1080 (American Type Culture Collection, ATCC) were cultured in EMEM (ATCC) supplemented with 10% FBS and 1% penicillin/streptomycin. HEK293T cells (ATCC) were maintained in DMEM (Gibco) supplemented with 10% FBS, 1% L-glutamine, and 1% penicillin/streptomycin. Genomic DNA was extracted from HEK293T cells using the Quick-DNA MiniPrep kit (Zymo). DU145 cells (ATCC) were cultured in RPMI-1640 (Gibco) supplemented with 10% FBS and 1% penicillin/streptomycin. MDA-MB-231 cells (ATCC) were cultured in DMEM (Gibco) supplemented with 10% FBS and 1% penicillin/streptomycin. TF1 and TF1 IDH2^{R140Q} knock-in cells (ATCC, CRL-2003 and CRL-20031G) were cultured in RPMI-1640 supplemented with L-glutamine, 10% FBS, 1% penicillin/streptomycin, and human GM-CSF (2 ng/mL, BioLegend) as recommended by ATCC. For pLVX stable line generation, TF1 cells were transduced with lentivirus from pLVX-IRES-neo vectors (Clontech #6321810) containing full length wild type or mutant (R140Q, R172K) IDH2 with a C-terminal Myc tag or empty vector and selected with 1 μg/mL geneticin (Gibco). For pSIN4 stable line generation, TF1 IDH2R^{140Q} knock-in cells were transduced with lentivirus from pSIN4-EF1a-TAL1-IRES-Puro (Addgene #61065) or empty vector generated via site-directed mutagenesis and selected with 2 μg/mL puromycin (Thermo Fisher Scientific). Cells were incubated at 37° C. and 5% CO₂.

Cloning and purification of recombinant proteins. Expression plasmids were acquired (pTXB1-Tn5, Addgene #60240) or cloned (APEX2 ORF from pTRC-APEX2, Addgene #72558) into the pTXB1 vector (NEB). Fusion constructs with different peptide linkers (Chen et al., Adv. Drug Deliv. Rev. 65:1357-1369, 2013) were generated by site-directed mutagenesis (NEB). All enzymes were expressed and purified similarly as previously described (Picelli et al., Genome Res. 24:2033-2040, 2014). In brief, plasmids were transformed into the Rosetta2 E. coli strain (EMD Millipore) and streaked out on an LB agar plate containing ampicillin and chloramphenicol. A single bacterial colony was inoculated into 10 mL LB with antibiotics and incubated overnight; this culture was then inoculated into 500 mL LB medium. Cultures were incubated at 37° C. until the optical density at 600 nm (OD600) reached ˜0.9. Isopropyl β-O-1-thiogalactopyranoside (IPTG) was added to a final concentration of 250 μM, cultures were incubated for 2 hours at 30° C., and bacteria were pelleted and frozen at −80° C.

Bacterial pellets were resuspended in 40 mL HEGX lysis buffer (20 mM HEPES-KOH pH 7.2, 1 M NaCl, 1 mM EDTA, 10% glycerol, 0.2% Triton X-100, 20 μM PMSF) and sonicated with a Sonic Dismembrator 100 (Fisher Scientific) at setting 7, with 5 pulses of 30 seconds on/off on ice. Lysate was spun at 15,000×g in a Beckman centrifuge (JA-10 rotor) for 30 minutes at 4° C. 1 mL 10% PEI was then added to the supernatant with constant agitation and clarified by centrifugation (15,000×g, 15 minutes, 4° C.). Supernatant was then applied to 5 mL chitin resin (NEB), prewashed with HEGX buffer, and incubated for 1 hour at 4° C. with agitation. Chitin slurry was applied to an Econo-Pak column (Bio-Rad) to remove unbound protein, washed with 20 column volumes of HEGX buffer and 1 column volume of HEGX with 50 mM DTT, and then incubated with 1 column volume of HEGX with 50 mM DTT for two days. After elution, the column was washed with 1 column volume of 2× dialysis buffer (2×DB: 100 mM HEPES-KOH pH 7.2, 0.2 M NaCl, 0.2 mM EDTA, 20% glycerol, 0.2% Triton X-100, 2 mM DTT). Eluates were combined, concentrated with a 10 kDa MWCO centrifugal filter, and subjected to buffer exchange with 2×DB using PD-10 desalting columns. Proteins were quantified via detergent-compatible Bradford assay (Thermo Fisher Scientific), snap frozen with liquid nitrogen, and stored at −80° C.

Transposome adaptor preparation. All transposome adaptors were synthesized at Thermo Fisher Scientific. The oligonucleotide sequences were similar as previously described (Chen et al., Nat. Methods 13:1013-1020, 2016; Picelli et al., Genome Res. 24:2033-2040, 2014): Tn5MErev, 5′-[phos]CTGTCTCTTATACACATCT-3′ (SEQ ID NO: 35); Tn5ME-A, 5′-TCGTCGGCAGCGTCAGATGTGTATAAGAGACAG-3′ (SEQ ID NO: 36); Tn5ME-B: 5′-GTCTCGTGGGCTCGGAGATGTGTATAAGAGACAG-3′ (SEQ ID NO: 37); Tn5ME-A-AF647, 5′-/AlexaFluor647/TCGTCGGCAGCGTCAGATGTGTATAAGAGACAG-3′ (SEQ ID NO: 36); Tn5ME-B-AF647: 5′-/AlexaFluor647/GTCTCGTGGGCTCGGAGATGTGTATAAGAGACAG-3′ (SEQ ID NO: 37). All oligos were resuspended in water to a final concentration of 200 μM each. Equimolar amounts of Tn5MErev/Tn5ME-A, Tn5MErev/Tn5ME-B, Tn5MErev/Tn5ME-A-AF647, and Tn5MErev/Tn5ME-B-AF647 were added together in separate tubes, denatured at 95° C. for 10 minutes, and cooled slowly to room temperature by removing the heat block. Tn5MEDS-A/Tn5MEDS-B and Tn5MEDS-A-AF647/Tn5MEDS-B-AF647 were combined at equimolar amounts to form 100 μM stocks of Tn5MEDS-A/B and Tn5MEDS-A/B-AF647, aliquoted, and stored at −20° C.

Electrophoretic mobility shift assay and DNA fragmentation analysis. pSMART HCAmp plasmid (Lucigen) was linearized with EcoRV-HF (NEB) and column-purified. DNA:protein complexes were assembled by incubating 12 pmol enzyme in 2×DB buffer with 15 pmol MEDS-A/B in water. 200 ng of linearized plasmid was then added to the enzyme mix and brought to a final volume of 20 μL containing 20% dimethylformamide, 20 mM Tris-HCl pH 7.5, and 10 mM MgCl2, with or without 50 mM EDTA. Tagmentation reactions were then incubated for 30 minutes at 37° C. For gel shift analysis, reactions were subjected to electrophoresis on a 1% agarose gel in Tris-acetate-EDTA (TAE) buffer, using gel loading dye without SDS (NEB). DNA fragmentation was assessed by adding SDS to a final concentration of 0.2% to the reaction mix after tagmentation and heated at 55° C. for 15 minutes. Reactions were then subjected to electrophoresis on a 1% agarose gel in TAE, using gel loading dye with SDS (NEB).

ATAC-seq/iDAPT-seq sample preparation. The OmniATAC sample preparation protocol was used similarly as previously described (Corces et al., Nat. Methods 14:959-962, 2017). 10 pmol enzyme (2 μL in 2×DB) was mixed with 12.5 pmol MEDS-A/B (1.25 μL in water) and incubated at room temperature for 1 hour. In the meantime, 50,000 cells were centrifuged at 500×g for 5 minutes at 4° C. Cells were resuspended in 50 μL lysis buffer 1 (LB1: 10 mM Tris-HCl pH 7.5, 10 mM NaCl, 3 mM MgCl2, 0.01% digitonin, 0.1% Tween-20, and 0.1% NP-40) with trituration, incubated on ice for 3 minutes, and then further supplemented with 1 mL lysis buffer 2 (LB2: 10 mM Tris-HCl pH 7.5, 10 mM NaCl, 3 mM MgCl2, and 0.1% Tween-20). Nuclei were pelleted (500×g, 10 minutes, 4° C.), resuspended with 50 μL tagmentation reaction mixture (20% dimethylformamide, 10 mM MgCl2, 20 mM Tris-HCl pH 7.5, 33% 1×PBS, 0.01% digitonin, 0.1% Tween-20, and 10 pmol enzyme equivalent of enzyme:DNA complex in 50 μL total volume), and incubated at 37° C. for 30 minutes with agitation on a thermomixer (1,000 rpm). Tagmentation with commercial Tn5 was performed as previously described (Corces et al., Nat. Methods 14:959-962, 2017). Tagmentation with naked genomic DNA was performed using 50 ng genomic DNA as substrate. After tagmentation, DNA libraries were extracted with DNA Clean and Concentrator-5 (Zymo) and eluted with 21 μL water.

To determine optimal PCR cycle number for library amplification, quantitative PCR was performed similarly as previously reported (Buenrostro et al., Nat. Methods 10:1213-1218, 2013). 2 μL of each ATAC-seq or iDAPT-seq library was added to 2×NEB Next Master Mix (NEB) and 0.4×SYBR Green (Thermo Fisher) with 1.25 μM of each primer (Primer 1: 5′-AATGATACGGCGACCACCGAGATCTACACTCGTCGGCAGCGTCAGATGTG-3′ (SEQ ID NO: 38); Primer 2.1: 5′-CAAGCAGAAGACGGCATACGAGATTCGCCTTAGTCTCGTGGGCTCGGAGATGT-3′ (SEQ ID NO: 39)) in a final volume of 15 μL, and quantification was assessed using the following conditions: 72° C. for 5 minutes; 98° C. for 30 seconds; and thermocycling at 98° C. for 10 seconds, 63° C. for 30 seconds and 72° C. for 1 minute. Optimal PCR cycle number was determined as the qPCR cycle yielding fluorescence between ¼ and ⅓ of the maximum fluorescence. The remaining DNA library was then amplified accordingly by PCR using previously reported barcoded primers for library multiplexing (Buenrostro et al., Nat. Methods 10:1213-1218, 2013), purified with DNA Clean and Concentrator-5 (Zymo), and eluted into 20 μL final volume with water. Libraries were then subject to TapeStation 2200 High Sensitivity D1000 fragment size analysis (Agilent) with NextSeq 500 High Output paired-end sequencing (2×75 bp, Illumina) as indicated.

ATAC-seq/iDAPT-seq data preprocessing. Paired-end sequencing reads were trimmed with TrimGalore v0.4.5, with adaptor sequence CTGTCTCTTATACACATCT (SEQ ID NO: 35) removed. Reads were aligned to the hg38 reference genome using bowtie2 v2.2.9 with options “--no-unal--no-discordant--no-mixed-X 2000.” Reads mapping to the mitochondrial genome were subsequently removed, and duplicate reads were removed with Picard v2.8.0. For insert size distribution, transcription start site (TSS) enrichment, and genome track visualization analyses, reads were downsampled to approximately 5 million paired-end fragments. Insert size distributions were determined by counting inferred fragment sizes from read alignments. TSS enrichment was performed by first shifting insert positions aligned to the reverse strand by −5 bp and to the forward strand by +4 bp as previously described (Buenrostro et al., Nat. Methods 10:1213-1218, 2013) and then determining the distance of each insertion to the closest Ensembl v94 transcription start site with Homer v4.9. Genic insertion preferences were similarly determined with Homer. Visualization was performed by mapping insertions to a genome-wide sliding 150 bp window with 20 bp offsets with bedops v2.4.30, followed by conversion to bigwig format with wigToBigWig from UCSC tools v363. Genome tracks were generated with Integrative Genomics Viewer v2.5.0.

Peaks were aligned by MACS2 v2.1.1 using options “callpeak--nomodel--shift-100--extsize 200--nolambda-q 0.01--keep-dup all,” generating either individual peak sets for each replicate (GM12878 analysis) or a consensus peak set after consolidating all reads (HEK293T, TF1 analyses). For GM12878 analysis, a union of all analyzed peaks was taken as a consensus peak set, and counts of insertions within peaks (downsampled to 5 million reads) were assessed using bedtools v2.26.0 with the multicov function. Correlation analysis was performed in R v3.5.0 using the pheatmap function. For HEK293T and TF1 analyses, consensus peaks overlapping with hg38 blacklist regions were removed (https://www.encodeproject.org/annotations/ENCSR636HFF/), and counts of insertions within peaks were assessed using the bedtools multicov function. Count matrices were processed with DESeq2 for differential insertions, and principal component analysis was performed with counts transformed with the varianceStabilizingTransformation function from DESeq2.

ATAC-seq/iDAPT-seq transcription factor analysis. Motif enrichment analysis was performed with ChromVAR as previously described using the human_pwms_v2 set of curated CisBP transcription factor motifs (Weirauch et al., Cell 158:1431-1443, 2014; Schep et al., Nat. Methods 14:975-978, 2017). ChromVAR motif deviations from the computeDeviations function were used for principal component analysis, and FDR-adjusted p-values were obtained with the differentialDeviations function with default settings.

Bivariate footprinting analysis was performed similarly as previously described with slight modifications (Corces et al., Science 362 (6413), 2018; Baek et al., Cell Rep. 19:1710-1722, 2017). Briefly, CisBP motifs within peaks were determined using matchMotifs from motifmatchr in R. Motif alignments were extended by 250 bp on each side, and adjusted transposon insertions were mapped to the corresponding regions. Motif flank height was determined by the average insertion rate between positions +1 to +50 bp, immediately flanking the motif. Background insertions were determined by the average insertion rate between positions +200 to +250 bp, distal to the positioned motif. Footprint height was determined by the 10% trimmed mean of the insertion rate within the 10-11 bp positioned around the center of the motif. Footprint depth (FPD) was determined as the log 2 of footprint height over flank height; flanking accessibility (FA) was determined as the log 2 of flank height over background. Because of the strong negative concordance between FA and FPD, we took the length of the orthogonal projection of FA and FPD scores onto the −45° line as a composite footprint score. Composite footprinting scores were modeled by a two-state Gaussian mixture model with mixtools, and enriched footprinted motifs were determined as those with greater than 50% probability of being in the Gaussian distribution further away from the origin.

For HEK293T analysis, gene expression detection in at least two of three mRNA-seq datasets (SRR5413179 (Zhang et al., Methods Mol. Biol. 1724:193-207, 2018), SRR5627161 (Altemose et al., Elife 6, 2017), and SRR6384877 (Shanmugam et al., Nucleic Acids Res. 46:7379-7395, 2018)) was used as a filtering criterion. Raw sequencing reads were aligned to a reference transcriptome generated with the Ensembl v94 database with salmon v0.13.1 using options “--seqBias--useVBOpt--gcBias--posBias--numBootstraps 30.” Length-scaled transcripts per million were acquired using the tximport function in R. Significant transcription factors were restricted to those with median read counts greater than 0 across the three independent mRNA-seq datasets.

ENCODE ChIP-seq transcription factor datasets were downloaded from the ENCODE data portal (Encode Consortium, Nature 489:57-74, 2012) (encodeproject.org). In brief, ChIP-seq bed files aligned to hg38 and annotated as “optimal IDR peaks” were downloaded, and iDAPT-seq peaks overlapping with ChIP-seq peaks were collated for enrichment analyses with iDAPT-seq datasets. For HEK293T peak enrichment, ChIP-seq enrichment was determined by Chi-squared test (with function chiseq.test in R) of a two-by-two contingency table corresponding to iDAPT-seq/ChIP-seq peak overlap within native chromatin peaks (DESeq2 FDR <5%, log 2 fold change >0, 18, 439 peaks) versus background peaks corresponding primarily to naked genomic DNA enrichment (log 2 fold change <0, 120, 182 peaks). For TF1 differential peak enrichment, ChIP-seq enrichment was determined by gene set enrichment analysis (GSEA) of differential peaks using the fgsea package in R, with peaks ranked by signed −log 10 p-values. GSEA plots were generated using a random sample of 2,000 ChIP-seq peaks for improved visualization.

Putative transcription factor interactions from iDAPT-seq were assessed by matching motifs with genomic positions using matchMotifs from motifmatchr and then performing hierarchical clustering on the resulting matrix with “binary” distance and “ward.D2” hierarchical clustering.

Co-immunofluorescence/ATAC-see analysis. ATAC-see was performed similarly as previously described with slight modifications (Chen et al., Nat. Methods 13:1013-1020, 2016). Enzyme and transposon DNA were mixed at a 1:1.25 enzyme:MEDS-A/B-AF647 molar ratio and incubated at room temperature for 1 hour. Adherent cells were grown on glass coverslips (Fisher Scientific, 12-540A) until 80-90% confluent, washed with 1×PBS, fixed with 1% formaldehyde (Electron Microscopy Services) in 1×PBS for 10 minutes, and washed twice with ice-cold 1×PBS. Suspension cells were washed and resuspended with 1×PBS. 50,000 cells were added to poly-lysine slides and incubated at room temperature for 1 hour in a humidified chamber. An equal volume of 2% formaldehyde was added and incubated for 10 minutes, whereupon slides were washed twice with ice-cold 1×PBS. Immobilized cells were lysed by incubation with LB1 for 3 minutes followed by LB2 for 10 minutes at room temperature. Cells were then subject to tagmentation (20% dimethylformamide, 10 mM MgCl2, 20 mM Tris-HCl pH 7.5, 33% 1×PBS, 0.01% digitonin, 0.1% Tween-20, and either 80 pmol enzyme equivalent of enzyme:DNA complex in a total volume of 100 μL for adherent cells or 10 pmol enzyme equivalent of enzyme:DNA complex in a total volume of 50 μL for suspension cells) for 30 minutes at 37° C. in a humidified chamber. Subsequently, cells were washed with 50 mM EDTA and 0.01% SDS in 1×PBS three times for 15 minute each at 55° C., lysed for 10 minutes with 0.5% Triton X-100 in 1×PBS at room temperature, and blocked with 1% BSA and 10% goat serum in PBS-T for 1 hour in a humidified chamber. Primary antibody was added to slides in 1% BSA/PBS-T and incubated at 4° C. overnight; slides were then washed and subjected to secondary antibody staining for 1 hour. Slides were washed with PBS-T three times for 15 minutes each, stained with DAPI (Sigma, 1 μg/mL) for 1 minute, washed with PBS for 10 minutes, and mounted with Fluorescence Mounting Medium (Dako). Confocal microscopy images were taken with an LSM 880 Axio Imager 2 at 63× magnification (Zeiss). Images were processed with Fiji/ImageJ v2.0.0.

Primary antibodies used were anti-RNA polymerase II CTD repeat YSPTSPS (phospho S2) (rabbit, Abcam ab5095, 1:500), anti-H3K27Ac (rabbit, Abcam ab4729, 1:500), anti-H3K9me3 (rabbit, Abcam ab8898, 1:500), anti-CCDCl2 (rabbit, Atlas Antibodies HPA060530, 1:200), anti-SNRPA (mouse, 3F9-1F7, Sigma-Aldrich WH0006626M1, 1:100). Secondary antibodies used were Goat anti-Rabbit IgG (H+L) Secondary Antibody, Alexa Fluor 488 conjugate (Thermo Fisher Scientific A11008, 1:1000) and Goat anti-Mouse IgG (H+L) Cross-Adsorbed Secondary Antibody, Alexa Fluor 488 conjugate (Thermo Fisher Scientific A11001, 1:1000).

Quantitative image analyses were performed with CellProfiler v3.1.5. Region of interests (ROIs) were identified from DAPI channel intensity values using minimum cross entropy thresholding, with each ROI corresponding to an individual nucleus. Pearson correlation coefficients were determined by comparing ATAC-see pixel intensities with corresponding immunofluorescence intensity values within each ROI to assess the nucleus-to-nucleus variation in colocalization.

Peroxidase activity assay. 5 pmol enzyme was incubated with 2.5 pmol hemin chloride (dissolved in DMSO, Cayman Chemical) for 1 hour at room temperature. This molar ratio was selected given reports of APEX2 maximal heme occupancy between 40-57%. Heme:protein complexes were then subjected to 50 μM Amplex UltraRed (Thermo Fisher Scientific) and 1 mM hydrogen peroxide for 1 minute at room temperature in a total volume of 100 μL with 1×PBS. Reactions were then quenched with 100 μL 2× quenching solution (10 mM Trolox, 20 mM sodium ascorbate, and 20 mM NaN₃ in 1×PBS), and fluorescence intensities were measured on a SpectraMax iD3 plate reader, with excitation at 530 nm and emission at 590 nm.

Western blot. Whole cell lysate was generated by resuspending cells washed with 1×PBS in RIPA (Boston BioProducts) supplemented with 1× complete EDTA-free protease inhibitor cocktail (Roche). Cells were subject to sonication via a Sonic Dismembrator 100 (Fisher Scientific) at setting 2, with 3 pulses of 15 seconds on/off on ice. Lysates were clarified by centrifugation (15,000×g, 30 minutes, 4° C.) and their concentrations quantified with detergent-compatible Bradford assay (Thermo Fisher Scientific). All Western blots were run on NuPAGE 4-12% Bis-Tris protein gels (Thermo Fisher Scientific) and transferred to 0.2 pm nitrocellulose membranes (GE Healthcare). Membranes were stained with Ponceau S, blocked with 3% milk in PBS-T, and incubated overnight with primary antibody and subsequently with secondary antibody after brief washing with PBS-T. Chemiluminescence was determined by applying ECL Western Blotting detection reagent (GE Healthcare) to membranes and imaging on an Amersham Imager 600 (GE Healthcare). Membranes were stripped with Restore PLUS Stripping Buffer (Thermo Fisher Scientific); streptavidin-HRP was inactivated with 15% hydrogen peroxide.

Primary antibodies used were anti-Myc-Tag (mouse, 9611, Cell Signaling Technology #2276, 1:1000), anti-IDH2 (rabbit, D8E3B, Cell Signaling Technology #56439, 1:1000), anti-H3K27me3 (rabbit, C36B11, Cell Signaling Technology #9733, 1:1000), anti-H3K9me3 (rabbit, Abcam ab8898, 1:5000), anti-α-tubulin (mouse, Sigma-Aldrich T6074, 1:4000), anti-FLAG M2 (mouse, Sigma-Aldrich, F1804, 1:2000), anti-TAL1 (rabbit, OriGene TA590662, 1:5000), and anti-HSP90 (mouse, 68, BD, BD Biosciences #610419, 1:2000). Secondary antibodies used were Rabbit IgG, HRP-linked F(ab′)2 fragment (GE Healthcare NA9340, from donkey, 1:5000) and Mouse IgG, HRP-linked whole Ab (GE Healthcare NA931, from sheep, 1:5000). Streptavidin-HRP (Cell Signaling Technology #3999S, 1:1000) was also used for probing.

Cytokine-independent growth. TF1 cells were washed three times with 1×PBS (150×g, 5 minutes) and then resuspended in RPMI supplemented with 10% fetal bovine serum and 1% penicillin/streptomycin at a density of 5e4 cells/mL in 10 mL. On each day of cell density measurement, 50 μL cell suspension was added to 50 μL CellTiter-Glo reagent, incubated for 10 minutes at room temperature, and assayed for luminescence with a SpectraMax iD3 plate reader.

Metabolite analysis. 5e6 cells were washed with 1×PBS (150×g, 5 minutes), resuspended in 800 μL prechilled 80% methanol, vortexed for 3 minutes, and frozen overnight at −80° C. Metabolites were extracted from the cell pellet three times with 80% methanol, with clarification via centrifugation (12,000 rpm, 15 minutes, 4° C.). The metabolite suspension was vacuum centrifuged to dryness, resuspended in HPLC-grade water, and analyzed by a targeted mass spectrometry-based metabolomic platform at the Beth Israel Deaconess Medical Center Mass Spectrometry Core Facility as previously described (Yuan et al., Nat. Protoc. 7:872-881, 2012).

Erythroid differentiation analysis. TF1 cells were processed as previously described (Losman et al., Science 339:1621-1625, 2013; Mugoni et al., Cell Res. doi:10.1038/s41422-019-0162-7, 2019). Cells were washed twice with plain RPMI and resuspended in RPMI supplemented with 10% fetal bovine serum and either 2 ng/mL GM-CSF (BioLegend) or 4 ng/mL erythropoietin (R&D) and 100 nM hemin chloride (Cayman Chemical). Media was refreshed every 3-4 days. Cells were analyzed after 12 days of culture by flow cytometry, washed with 2% fetal bovine serum prior to staining. Anti-CD235a-FITC antibody conjugate (mouse, HI264, BioLegend #349108) was incubated with samples for 15 minutes and then washed to remove excess antibody. Stained samples were analyzed on a Beckman Coulter Gallios flow cytometer.

GATA1/TAL1 proximal gene signature analysis. Preprocessed TCGA LAML mRNA-seq HTSeq gene counts were downloaded through TCGABiolinks in R, and IDH1/2 mutation status was obtained from cBioPortal (http://www.cbioportal.org/). Differential gene expression was assessed with DESeq2, regressing on IDH1/2 mutation status with no additional covariates, and resultant signed −log 10 p-values were used to rank genes for GSEA. A GATA1/TAL1 proximal gene signature was assembled by determining ChIP-seq peak overlap between the two proteins within differentially inaccessible peaks from TF1 mIDH2 analysis (DESeq2 p-value <0.05, log 2 fold change <0). The nearest Ensembl gene to each peak was determined by Homer, removing peaks annotated as intergenic. GSEA was performed with fgsea in R.

DNA and protein tagging by iDAPT. iDAPT with HEK293T cells: 5 μmol MEDS-A/B, 4 μmol enzyme, and 2 μmol hemin chloride per channel were incubated at room temperature for 1 hour. HEK293T cells were trypsinized and washed with 1×PBS. 2e8 cells were pelleted (500×g, 5 minutes, 4° C.), lysed with 500 μL LB1 with 1× cOmplete EDTA-free protease inhibitor cocktail (Roche) and PhosSTOP phosphatase inhibitor (Roche) for 3 minutes, and further supplemented with an additional 10 mL of LB2 with protease and phosphatase inhibitors. 2e7 nuclei per channel were aliquoted into separate tubes, pelleted (500×g, 10 minutes, 4° C.), and resuspended with tagmentation reaction mixture (20% dimethylformamide, 10 mM MgCl2, 20 mM Tris-HCl pH 7.5, 33% 1×PBS, 0.01% digitonin, 0.1% Tween-20, 500 μM biotin phenol, 1× protease and phosphatase inhibitors, and 4 μmol enzyme equivalent of enzyme:DNA:heme complex in a total volume of 1 mL), and incubated at 37° C. for 30 minutes with agitation on a thermomixer (1,000 rpm). 2.5 μL of tagmentation mix was saved for library preparation and quality assessment as described above for ATAC-seq sample preparation. The remaining nuclear suspension was then washed with 1×PBS supplemented with biotin phenol and protease and phosphatase inhibitors, and labeled with 1 mM hydrogen peroxide and biotin phenol for 1 minute. Peroxidation reactions were quenched with 2× quenching buffer (20 mM NaNs, 10 mM Trolox, 20 mM sodium ascorbate with protease and phosphatase inhibitors). Labeled nuclei were then pelleted, washed with 1× quenching buffer, and resuspended in 500 μL RIPA containing protease and phosphatase inhibitors. Nuclear suspension was sonicated (setting 2, 10 seconds, 3 pulses), 1 μL of benzonase was added to the suspension, and the lysate was clarified by centrifugation (15,000×g, 20 minutes, 4° C.). 500 μg lysate was reduced with DTT at a final concentration of 5 mM and then added to 30 μL Pierce streptavidin beads washed 2× with RIPA buffer. The lysate/bead mixture was incubated with end-to-end rotation for 3 hours at 4° C. Beads were washed 3× with RIPA and 2× with 200 mM EPPS pH 8.5. Beads were resuspended with 100 μL 200 mM EPPS pH 8.5, 1 μL mass spectrometry-grade trypsin was added, and samples were incubated overnight at 37° C. with mixing. Beads were magnetized, and eluate was collected and subjected to downstream tandem mass tag (TMT) labeling.

iDAPT with TF1 cells: 2.5 μmol MEDS-A/B, 2 μmol enzyme, and 1 μmol hemin chloride per channel were incubated at room temperature for 1 hour. 1e7 cells per channel were washed (500×g, 5 minutes, 4° C.), lysed with 100 μL LB1 with 1× cOmplete EDTA-free protease inhibitor cocktail (Roche) and PhosSTOP phosphatase inhibitor (Roche) for 3 minutes, and further supplemented with an additional 1 mL of LB2 with protease and phosphatase inhibitors. Nuclei were pelleted (500×g, 10 minutes, 4° C.), and resuspended with tagmentation reaction mixture (20% dimethylformamide, 10 mM MgCl2, 20 mM Tris-HCl pH 7.5, 33% 1×PBS, 0.01% digitonin, 0.1% Tween-20, 500 μM biotin phenol, 1× protease and phosphatase inhibitors, and 2 μmol enzyme equivalent of enzyme:DNA:heme complex in a total volume of 1 mL), and incubated at 37° C. for 30 minutes with agitation on a thermomixer (1,000 rpm). 5 μL of tagmentation mix was saved for library preparation and quality assessment as described above for ATAC-seq sample preparation. The remaining nuclear suspension was then washed with 1×PBS supplemented with biotin phenol and protease and phosphatase inhibitors, and labeled with 1 mM hydrogen peroxide and biotin phenol for 1 minute. Peroxidation reactions were quenched with 2× quenching buffer. Labeled nuclei were then pelleted, washed with 1× quenching buffer, and resuspended in 250 μL RIPA containing protease and phosphatase inhibitors. Nuclear suspension was sonicated (setting 2, 10 seconds, 3 pulses), 1 μL of benzonase (EMD Millipore) was added to the suspension, and the lysate was clarified by centrifugation (15,000×g, 20 minutes, 4° C.). 250 μg lysate was reduced with DTT at a final concentration of 5 mM and then added to 30 μL Pierce streptavidin beads washed 2× with RIPA buffer. Lysate/bead mixture was incubated with end-to-end rotation for 3 hours at 4° C. Beads were washed 3× with RIPA, 2× with 200 mM EPPS pH 8.5, and resuspended with 100 μL 200 mM EPPS pH 8.5. 1 μL MS-grade lysC was added to each tube and incubated at 37° C. for 3 hours with mixing, and an additional 1 μL mass spectrometry-grade trypsin was added, followed by overnight incubation at 37° C. with mixing. Beads were magnetized, and eluate was collected and subjected to downstream TMT labeling.

Tandem mass tag labeling. Peptides were processed using the SL-TMT method (Navarrete-Perea et al., J. Proteome Res. 17:2226-2236, 2018). TMT reagents (0.8 mg) were dissolved in anhydrous acetonitrile (40 μL), of which 10 μL was added to the peptides (100 μL) with 30 μL of acetonitrile to achieve a final acetonitrile concentration of approximately 30% (v/v). Following incubation at room temperature for 1 hour, the reaction was quenched with hydroxylamine to a final concentration of 0.3% (v/v). The TMT-labeled samples were pooled at a 1:1 ratio across all samples. The pooled sample was vacuum centrifuged to near dryness and subjected to C18 solid-phase extraction (SPE) (Sep-Pak, Waters).

Off-line basic pH reversed-phase (BPRP) fractionation. We fractionated the pooled TMT-labeled peptide sample using BPRP HPLC (Wang et al., Proteomics 11:2019-2026, 2011). We used an Agilent 1200 pump equipped with a degasser and a photodiode array (PDA) detector (set at 220 and 280 nm wavelength) from ThermoFisher Scientific (Waltham, Mass.). Peptides were subjected to a 50-min linear gradient from 9% to 35% acetonitrile in 10 mM ammonium bicarbonate pH 8 at a flow rate 600 μL/min over an Agilent 300Extend C18 column (3.5 pm particles, 4.6 mm ID and 220 mm in length). The peptide mixture was fractionated into a total of 96 fractions, which were consolidated into 24 (Paulo et al., J. Proteomics 148:85-93, 2016). Samples were subsequently acidified with 1% formic acid and vacuum centrifuged to near dryness. Each consolidated fraction was desalted via StageTip, dried again via vacuum centrifugation, and reconstituted in 5% acetonitrile, 5% formic acid for LC-MS/MS processing.

LC-MS/MS proteomic analysis. Samples were analyzed on an Orbitrap Fusion mass spectrometer (Thermo Fisher Scientific, San Jose, Calif.) coupled to a Proxeon EASY-nLC 1200 liquid chromatography (LC) pump (Thermo Fisher Scientific). Peptides were separated on a 100 pm inner diameter microcapillary column packed with 35 cm of Accucore C18 resin (2.6 pm, 150 Å, ThermoFisher). For each analysis, approximately 2 μg of peptides were separated using a 75 minute gradient of 8 to 28% acetonitrile in 0.125% formic acid at a flow rate of 450-500 nL/minute. Each analysis used an MS3-based TMT method (Ting et al., Nat. Methods 8:937-940, 2011; McAlister et al., Anal. Chem. 86:7150-7158, 2014), which has been shown to reduce ion interference compared to MS2 quantification (Paulo et al., J. Am. Soc. Mass Spectrom. 27:1620-1625, 2016). The scan sequence began with an MS1 spectrum (Orbitrap analysis, resolution 120,000, 350-1400 Th, automatic gain control (AGC) target 2e5, maximum injection time 100 ms). The top ten precursors were then selected for MS2/MS3 analysis. MS2 analysis consisted of: collision-induced dissociation (CID), quadrupole ion trap analysis, automatic gain control (AGC) 1.4e4, NCE (normalized collision energy) 35, q-value 0.25, maximum injection time 120 ms), and isolation window at 0.7. Following acquisition of each MS2 spectrum, we collected an MS3 spectrum in which multiple MS2 fragment ions are captured in the MS3 precursor population using isolation waveforms with multiple frequency notches. MS3 precursors were fragmented by HCD and analyzed using the Orbitrap (NCE 65, AGC 1.5e5, maximum injection time 150 ms, resolution was 50,000 at 400 Th).

Proteomic data analysis. Mass spectra were processed using a Sequest-based pipeline (Huttlin et al., Cell 143:1174-1189, 2010). Spectra were converted to mzXML using a modified version of MSConvert. Database searching included all entries from the human UniProt database. This database was concatenated with one composed of all protein sequences in the reversed order. Searches were performed using a 50-ppm precursor ion tolerance for total protein level analysis. The product ion tolerance was set to 0.9 Da. TMT tags on lysine residues and peptide N termini (+229.163 Da) and carbamidomethylation of cysteine residues (+57.021 Da) were set as static modifications, while oxidation of methionine residues (+15.995 Da) was set as a variable modification.

Peptide-spectrum matches (PSMs) were adjusted to a 1% false discovery rate (FDR) (Elias et al., Methods Mol. Biol. 604:55-71, 2010; Elias et al., Nat. Methods 4:207-214, 2007). PSM filtering was performed using a linear discriminant analysis (LDA), as described previously (Huttlin et al., Cell 143:1174-1189, 2010), while considering the following parameters: XCorr, ΔCn, missed cleavages, peptide length, charge state, and precursor mass accuracy. For TMT-based reporter ion quantitation, we extracted the summed signal-to-noise (S:N) ratio for each TMT channel and found the closest matching centroid to the expected mass of the TMT reporter ion. For protein-level comparisons, PSMs were identified, quantified, and collapsed to a 1% peptide false discovery rate (FDR) and then collapsed further to a final protein-level FDR of 1%, which resulted in a final peptide level FDR of <0.1%. Moreover, protein assembly was guided by principles of parsimony to produce the smallest set of proteins necessary to account for all observed peptides. PSMs with poor quality, MS3 spectra with more than eight TMT reporter ion channels missing, MS3 spectra with TMT reporter summed signal-to-noise of less than 100, missing MS3 spectra, or isolation specificity <0.7 were excluded from quantification (McAlister et al., Anal. Chem. 84:7469-7478, 2012).

PSM intensities were quantile normalized and log 2-transformed. Transformed PSM intensities were collapsed to proteins by arithmetic average, with priority given to uniquely mapping peptides. Principal component analysis was performed at the protein quantitation level. The limma package in R was used to determine differential protein abundances.

Protein enrichment analyses. ReactomeDB pathway to gene mappings were obtained with the reactomePathways function from fgsea. For HEK293T analysis, the enricher function from clusterProfiler in R was used to determine pathway enrichment above background. Background proteins were genes with corresponding UniProt Ds and ensembl gene IDs in biomaRt.

Gene Ontology terms were selected from the Human Protein Atlas (http://www.proteinatlas.org/) to represent well-defined subcellular localization patterns. Gene to Gene Ontology mappings were determined from org.Hs.eg.db in R. Subcellular localization analyses were performed using the enricher function from clusterProfiler. Open chromatin proteomic enrichment datasets were compiled (REFs) and harmonized to UniProt IDs, and FDR-adjusted p-values were quantile normalized and then subjected to −log 10 transformation to diminish technical differences in proteomic detection strategies across studies.

Using significant sequence-specific transcription factors from HEK293T iDAPT-MS, we identified first-order protein interactors and their connections from BioPlex (REF). CORUM protein complex information (version 3.0) was downloaded, and annotated protein complex enrichment was performed using the enricher function from clusterProfiler in R.

For TF1 iDAPT-MS analysis, signed −log 10 p-values from limma were used to rank proteins for gene set enrichment analysis via fgsea. ReactomeDB pathway gene sets were used as described above. R-2HG protein targets were collated from Losman et al., Genes Dev. 27:836-852, 2013, and multi-validated BioGrid (Oughtred et al., Nucleic Acids Res. 47:D529-D541, 2019) ego-centric physical protein complexes (version 3.5.166) were downloaded (https://thebiogrid.org/).

Open chromatin marker analysis. Open chromatin marker analysis was performed as described in the main text. Gene Ontology subcellular annotation was performed as described above. The BioPlex interactome (Huttlin et al., Nature 545:505-509, 2017; Huttlin et al., Cell 162:425-440, 2015) (version 2.3) was downloaded (http://bioplex.hms.harvard.edu/) and filtered to include only vertices corresponding to the proteins enriched by TP3 in HEK293T cells. Network analyses were performed with the igraph package in R. The Cancer Cell Line Encyclopedia (Ghandi et al., Nature doi:10.1038/s41586-019-1186-3, 2019; Barretina et al., Nature 483:603-607, 2012) gene expression TPM matrix (version 18q4) was downloaded (https://depmap.org/portal/), and coefficient of variance was determined for each gene.

Statistical analysis. All statistical analyses were performed in R. Two-tailed statistical tests were used as described. Multiple comparison adjustments were performed as noted.

### Example 2

### Introduction and Results

In additional studies, we further analyzed data described above. We also carried out experiments using two leukemia cell lines: K562 and NB4. In addition, we carried out studies of how the open chromatin landscape changes in response to differentiation stimuli. Furthermore, we demonstrate the platform as an approach to infer what is happening from a global perspective based on the proteomic and genomic data obtained. For example, we show that one can infer what proteins may be doing based on where they fall in a plot, e.g., whether they are activators or repressors, and thereby assign a level of function to them.

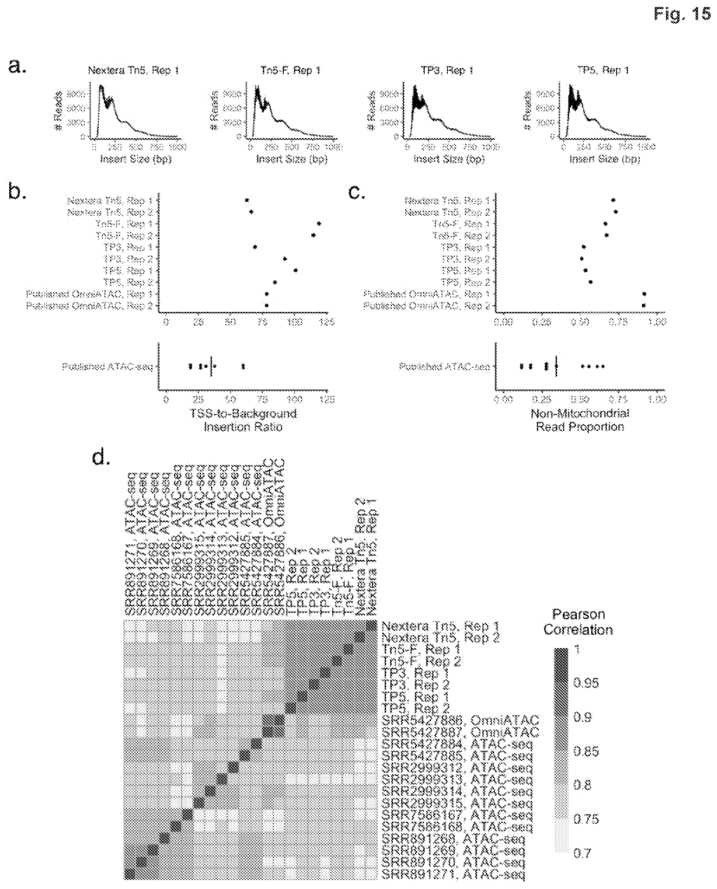

As explained above in reference to FIG. 1a, we distinguished iDAPT-seq from ATAC-seq with the use of TP fusion enzymes for tagmentation, allowing for subsequent proteomic labeling and enrichment (FIG. 1a). ATAC-seq and iDAPT-seq libraries exhibited similar nucleosomal periodicities in their fragment size distributions, high signal-to-noise ratios, and broad decreases in mitochondrial read proportions relative to published GM12878 ATAC-seq libraries generated via the original ATAC-seq protocol (see above) (FIG. 15a-15c). Furthermore, as noted above, TP3 and TP5 iDAPT-seq libraries exhibit high correlations with Tn5 transposase-generated ATAC-seq libraries (FIGS. 1b and 1c, FIG. 15d). Thus, TP3 and TP5 fusion enzymes yield high quality iDAPT-seq libraries, akin to ATAC-seq libraries generated via Tn5 transposase enzyme lacking a peroxidase domain.

As explained above, as a further assessment of TP localization to open chromatin, we performed ATAC-see with co-immunofluorescence of markers of chromatin state. TP3 and Tn5-F exhibit similarly positive correlations with histone H3 lysine 27 acetylation (H3K27Ac) and RNA polymerase II serine-2 phosphorylation (RNAPII S2P) immunofluorescence signals, and similarly poor correlations with H3 lysine 9 trimethylation (H3K9me3) immunofluorescence, albeit with slight differences in colocalization patterns between the two probes (FIG. 1d-e). These data show that our TP fusion probes retain native Tn5 transposase activity and preferentially tag open chromatin.

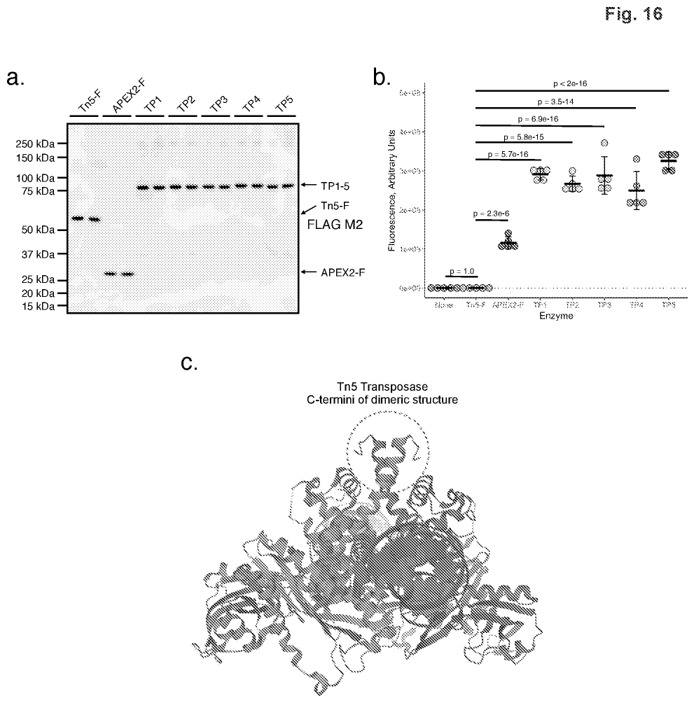

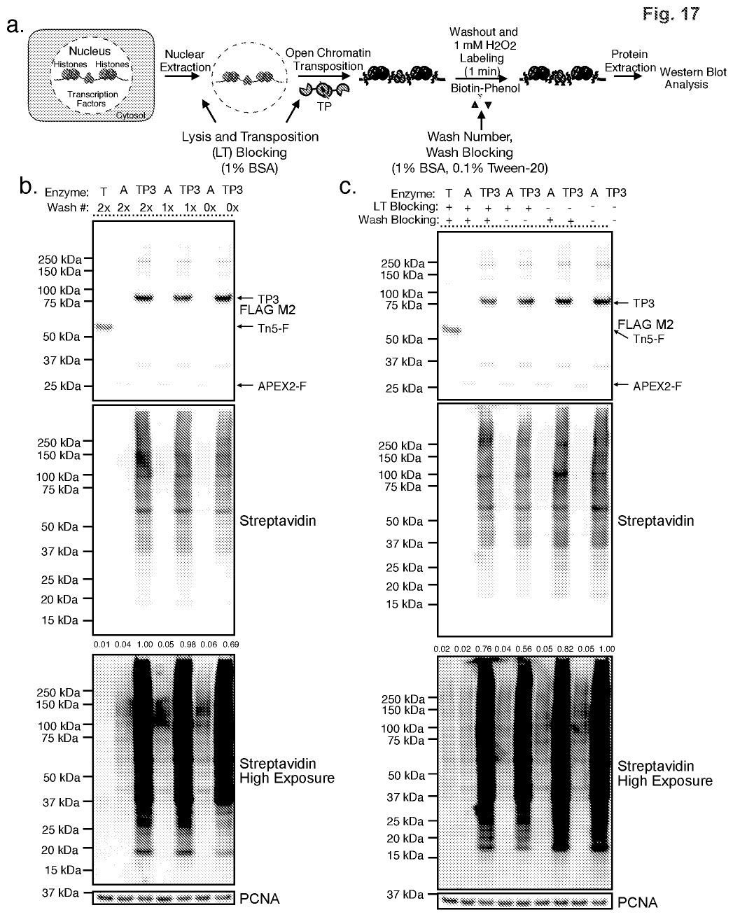

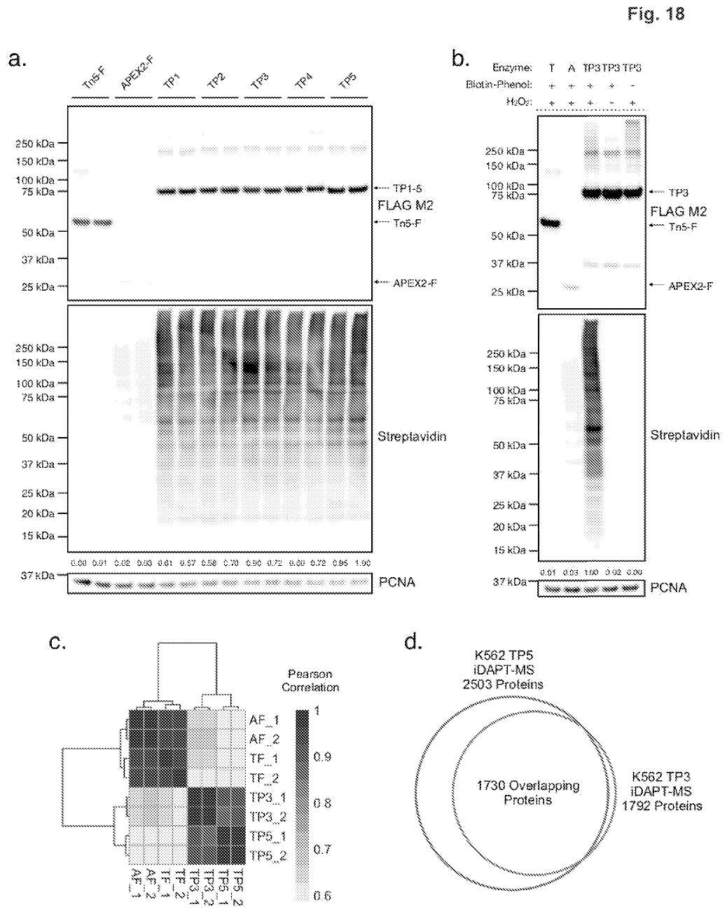

Having confirmed TP fusion tagging of and localization to open chromatin, we assessed APEX2 peroxidase functionality when fused with Tn5 transposase, as explained above. First to confirm this, we added 1 mM hydrogen peroxide to purified proteins alone and detected peroxidase activity from the fusion proteins via resorufin fluorescence after one minute (FIGS. 16a and 16b). All TP fusions exhibit higher peroxidase activities than APEX2-F alone, possibly due to increased thermal stability or heme binding of APEX2 dimer formation induced by the proximity of the two C-termini of dimeric Tn5 transposase, as noted above (FIG. 16c). Next, as noted above, in extracted HEK293T nuclei, we observed strong peroxidase-dependent biotin signal in the presence of the TP3 fusion probe and low signal in the presence of the negative control probes Tn5-F and APEX2-F (FIG. 17). Residual APEX2-F-mediated signal further decreased with additional washing and blocking steps while maintaining strong TP3-mediated biotin signal (FIG. 17). In line with our hypothesis that Tn5 transposase remains physically bound to native chromatin, Tn5 transposase and TP3 fusion enzyme are found in the nuclear lysate, whereas APEX2 is mostly lost despite equimolar addition of recombinant protein to the tagmentation buffer (FIGS. 16a, 17b, and 17c). Indeed, we found all TP fusion enzymes to promote strong biotin labeling in K562 nuclei, with TP5 and TP3 enzymes exhibiting the highest levels of labeling (FIG. 18a). Finally, we confirmed that this labeling is dependent on the presence of both hydrogen peroxide and biotin-phenol (FIG. 18b). Thus, our findings indicate that TP probes label transposase-accessible chromatin in a peroxidase-dependent manner.

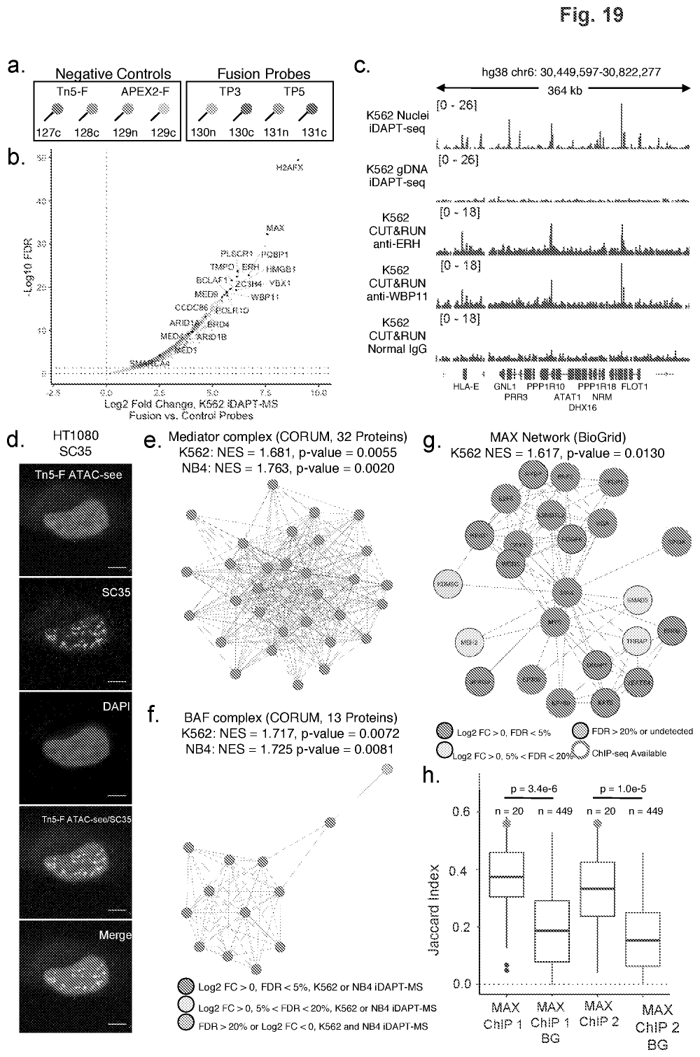

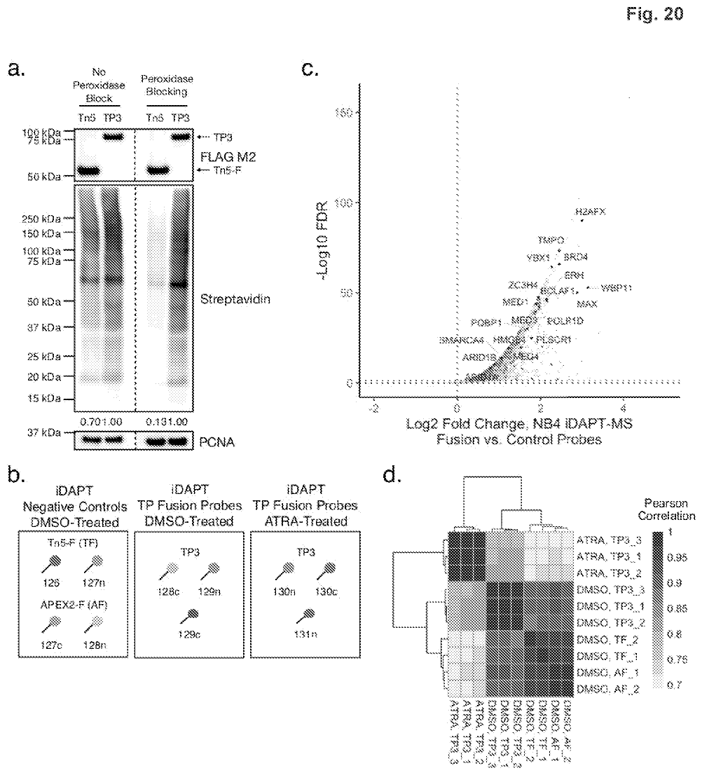

With our optimized iDAPT protocol, we performed quantitative mass spectrometry on the iDAPT-enriched proteome (iDAPT-MS) from K562 nuclei (Navarrete-Perea et al., J. Proteome Res. 17:2226-2236, 2018) (FIG. 19a). As negative control probes enrich for nonspecific background signal, akin to an IgG negative control for an immunoprecipitation assay, we interpreted the substantial proteomic content enriched by TP over negative control probes as bona fide proteins proximal to Tn5 transposase localization in isolated nuclei (FIG. 19b). By hierarchical clustering and correlation analyses, nuclear lysates labeled via TP3 and TP5 segregate from lysates labeled via single enzymatic domains, with substantial overlap between TP3- and TP5-enriched proteomes (FIGS. 18a-18c). We observed a similarly substantial iDAPT-MS enrichment pattern from TP3 versus negative control probes from the NB4 cell line, incorporating an additional wash step to block endogenous peroxidase activity prior to tagmentation and biotin labeling (FIG. 20).

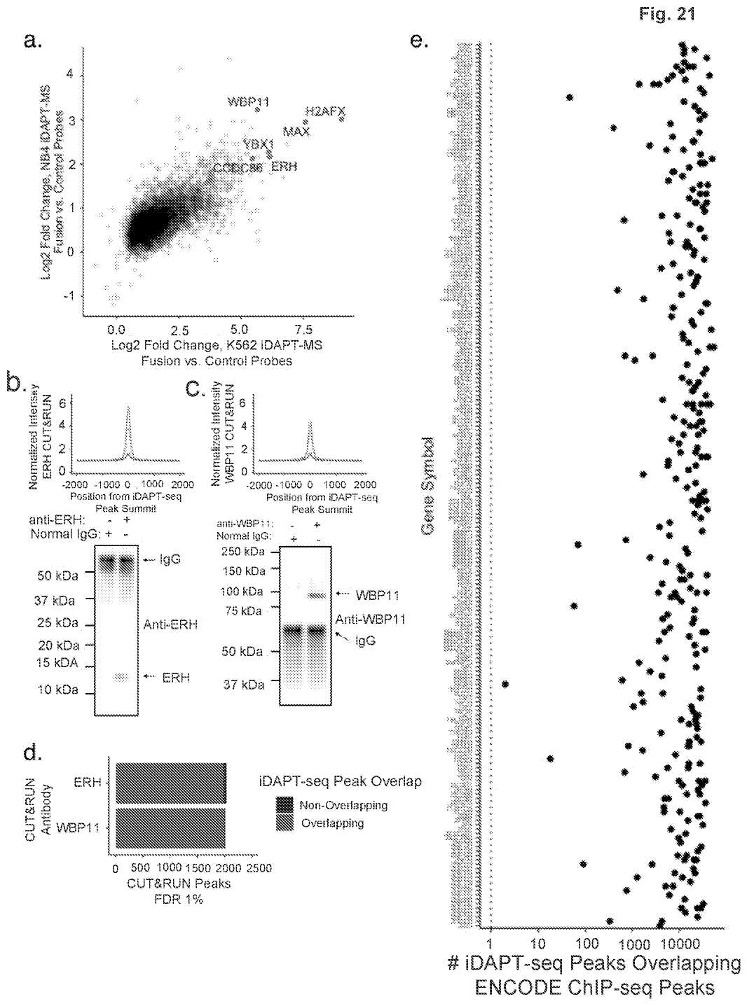

To validate highly enriched proteins by iDAPT-MS, we performed CUT&RUN (ERH and WBP11) and analyzed published ENCODE ChIP-seq datasets from the K562 cell line (Encode Consortium, Nature 489:57-74, 2012; Skene et al., Elife 6, 2017). We found substantial enrichment of protein binding at sites of open chromatin (FIGS. 19c and 21). These results further demonstrate the ability of iDAPT-MS to discover proteins associated with open chromatin.

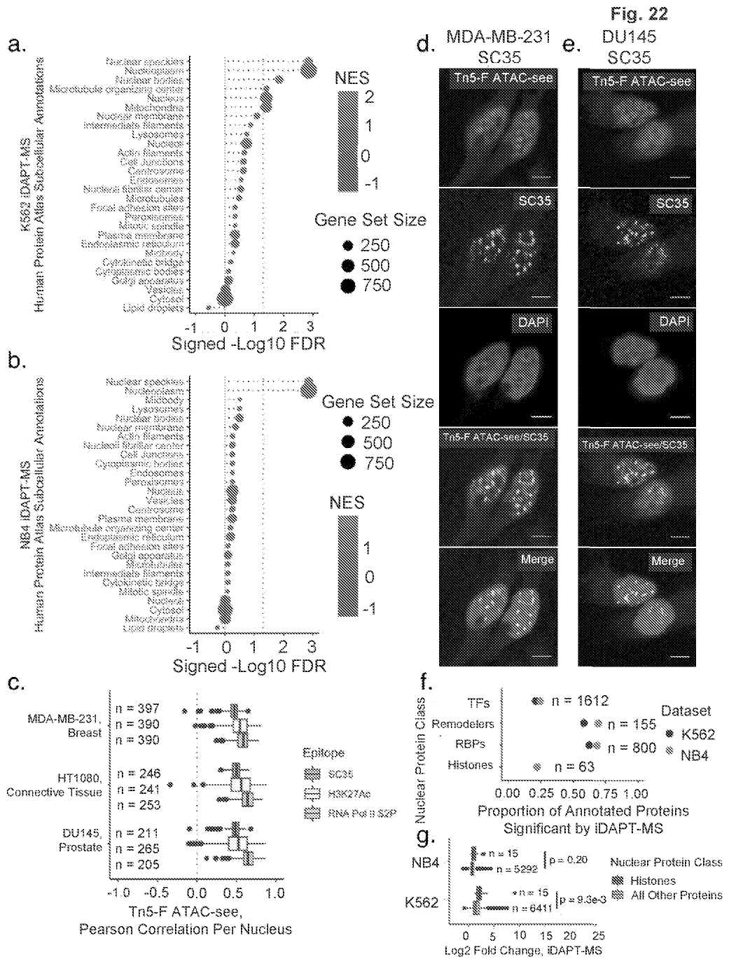

We further performed enrichment analyses of our iDAPT-MS datasets. Subcellular enrichment analysis identified nuclear speckles and nucleoplasm in both K562 and NB4 iDAPT-MS datasets (Thul et al., Science 80:356, eaa13321, 2017) (FIGS. 22a and 22b). Indeed, ATAC-see signal of Tn5-F colocalizes with the nuclear speckle marker SC35 in multiple cell lines, in agreement with recent reports of nuclear speckle localization at active promoters (Xiao et al., Cell 178:107-121.e18, 2019, Guo et al., Nature 572:543-548, 2019) (FIGS. 19d and 22c-22e). We further identified significant enrichment of protein complexes such as Mediator, which regulates communication from enhancer- and promoter-bound transcription factors to RNA polymerase II (Allen et al., Nat. Rev. Mol. Cell Biol. 16:155-166, 2015), and BAF, which remodels chromatin accessibility (Kadoch et al., Sci. Adv. 1:1-18, 2015), in both K562 and NB4 cell lines (Ruepp et al., Nuc. Acids Res. 36:D646-D650, 2008) (FIGS. 19e and 19f). Chromatin remodelers and RNA-binding proteins were highly represented (>50% of annotated proteins) among enriched proteins, whereas transcription factors and histone variants were not as well represented (<25% of annotated proteins) (FIG. 220. While histone protein H2AX/H2AFX was highly enriched in both NB4 and K562 iDAPT-MS proteomes, other detected histone proteins were weakly enriched over negative control probes or not detected, suggesting that histone proteins as a class are not predominantly enriched by iDAPT-MS (FIGS. 19b, 20c, and 22f-g).

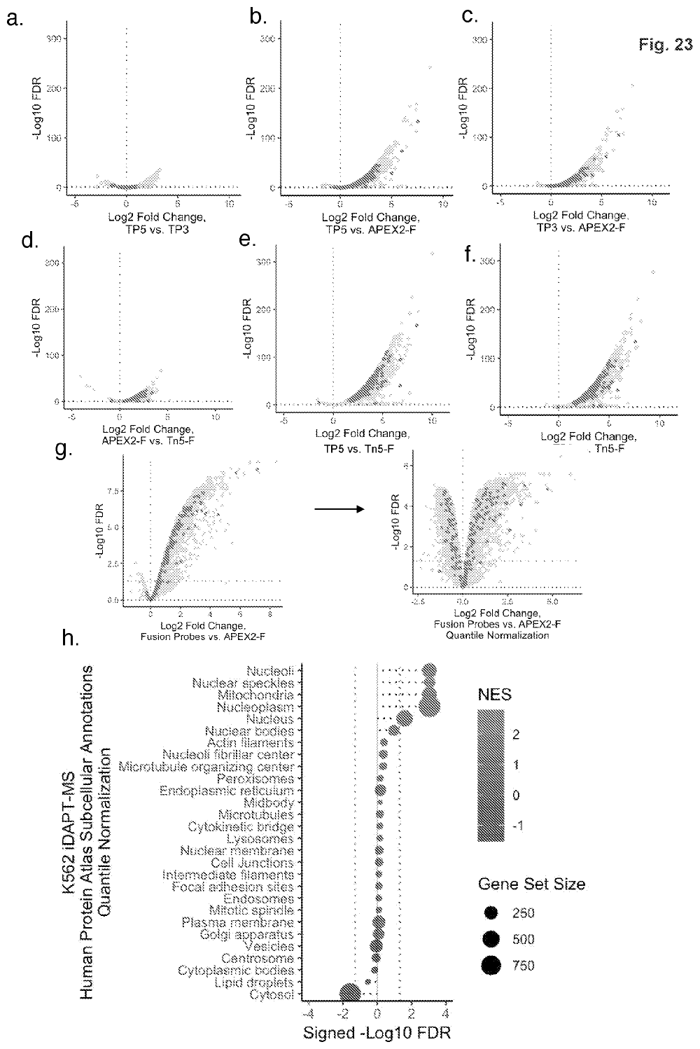

Despite low background peroxidase signal, APEX2-F yields some proteomic enrichment over Tn5-F, although not as strongly as signal generated by TP3/TP5 (FIGS. 23a-23f). To assess whether APEX2-F has a different labeling propensity over TP3/TP5 fusion probes in K562 nuclei, we used quantile normalization as a proxy for normalizing APEX2-F peroxidase activity with TP3 and TP5 activities (FIG. 23g). We found this quantile normalization scheme to yield similar subcellular enrichment patterns, albeit with increased mitochondrial enrichment, as with our primary streptavidin/trypsin peptide normalization scheme (FIGS. 22a and 23h). Taken together, these data suggest that TP fusion proteins exhibit different labeling patterns from diffusely nuclear APEX2.

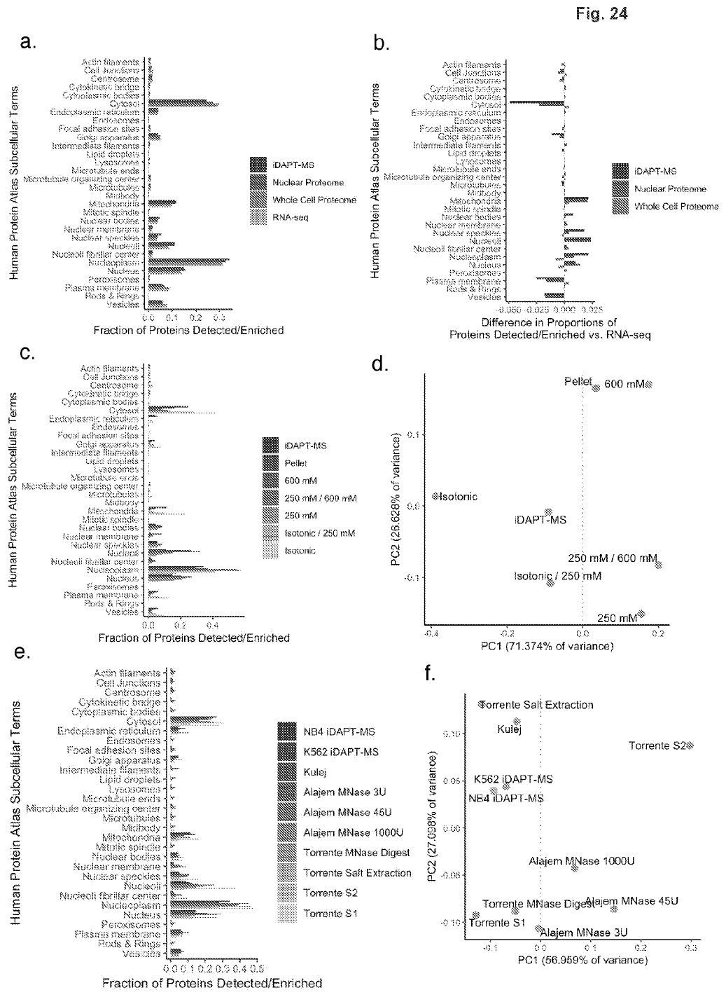

We compared iDAPT-MS enrichment relative to other techniques used to assess protein abundance on chromatin. First, we collated sets of detected proteins from K562 RNA-seq (protein-coding transcripts) (Encode Consortium, Nature 489:57-74, 2012), whole cell proteome (Nusinow et al., Cell 180:387-402.e16, 2020), and nuclear proteome (Federation et al., Cell Rep. 30:2463-2471.e5, 2020) datasets and then assessed the proportions of proteins detected across subcellular compartments in each of these datasets to normalize for proteome complexity. While we observed mild subcellular enrichment differences between RNA-seq and whole cell proteome datasets, we found increased enrichment of nucleoli, nucleoplasm, and nucleus localization terms from iDAPT-MS and nuclear proteome datasets (FIGS. 24a and 24b). The K562 iDAPT-MS-enriched proteome exhibits increased enrichment of nuclear speckles, nucleoplasm, and nuclear body localization terms and decreased cytosolic, plasma membrane, and Golgi apparatus localization terms over the nuclear proteome (FIG. 24b). Second, we assessed how iDAPT-MS enrichment compares with incremental salt extractions from K562 nuclei, partitioning euchromatic and heterochromatic proteins via disrupting electrostatic protein-protein and protein-DNA interactions (Federation et al., Cell Rep. 30:2463-2471.e5, 2020) (FIGS. 24c and 24d). After converting protein sets to subcellular enrichment scores and performing principal component analysis, we found that K562 iDAPT-MS coincides with proteins identified by both isotonic and 250 mM salt extractions along the first principal component, largely representing euchromatic proteins. Third, we compared iDAPT-MS enrichment with additional published salt extraction- and micrococcal nuclease (MNase) fragmentation-based chromatin proteomic datasets in a similar manner (Torrente et al., PLoS One 6:e24747, 2011; Alajem et al., Cell Rep. 10:2019-2031, 2015; Kuleg et al., Mol. Cell. Prot. 16:S92-S107, 2017) (FIGS. 24e and 24f). Indeed, iDAPT-MS enrichment corresponds with chromatin proteomes enriched by light MNase digestion and salt extraction along the first principal component. Together, these findings demonstrate that iDAPT-MS enriches for the open chromatin proteome.

A critical advantage of iDAPT-MS over ATAC-seq/iDAPT-seq or chromatin immunoprecipitation (ChIP)-based approaches is its ability to capture numerous transcription co-factors associated with open chromatin in a single assay, which regulate their associated sequence-specific transcription factors. As proof of principle, we found the MAX protein interaction network to be significantly enriched on open chromatin by K562 iDAPT-MS (Oughtred et al., Nuc. Acids Res. 47:D529-D541, 2019) (FIG. 19g). To validate this finding, by ChIP-seq analysis, protein interactors of MAX colocalize more tightly with MAX across the open chromatin landscape than do non-interacting proteins (FIG. 19h). Therefore, iDAPT-MS together with protein interaction annotations facilitates the identification of active transcription factor protein complexes on open chromatin, expanding the inference of cis-regulatory transcription factor networks.

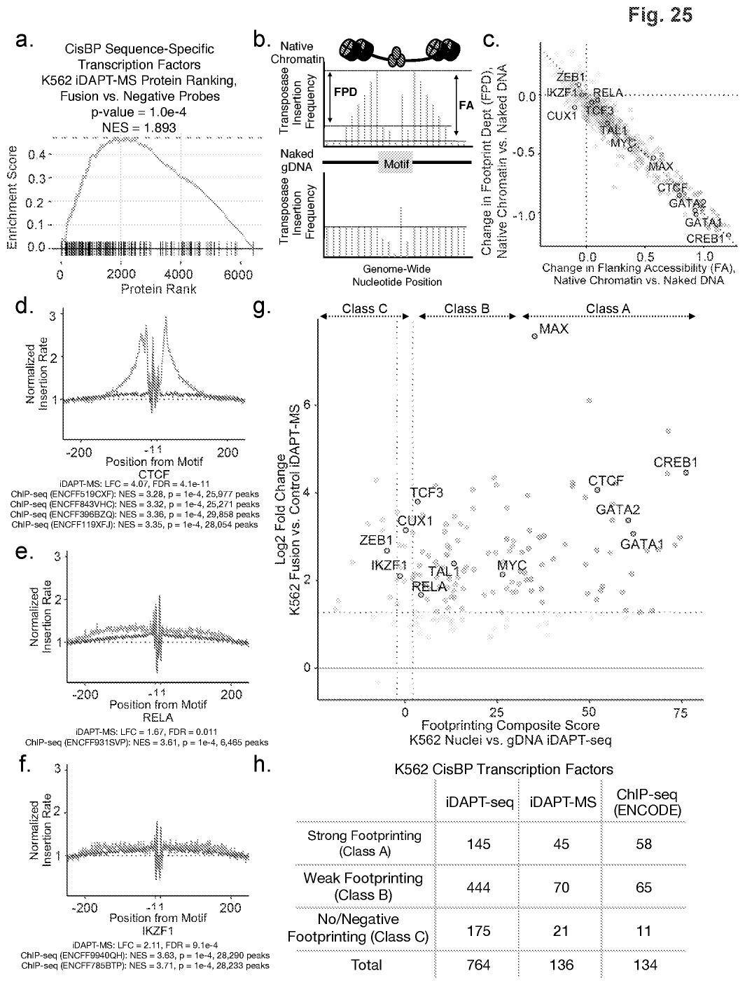

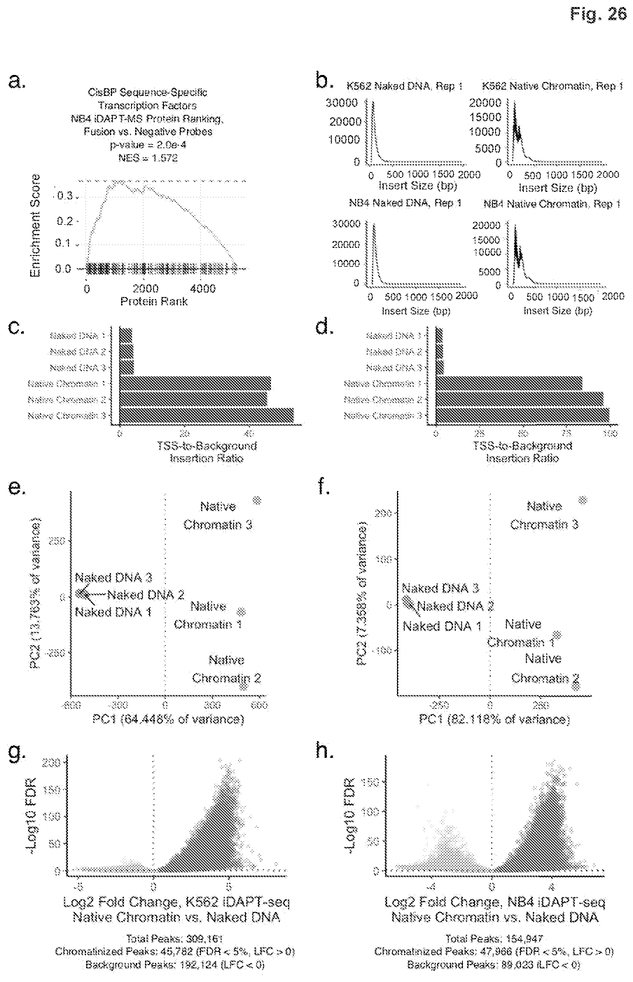

Transcription factors regulate gene expression by binding to DNA in a sequence-specific manner and recruiting transcriptional activators and/or repressors to their target genes. Most transcription factors are found within regions of open chromatin, a pattern we also observed in our iDAPT-MS data (Lambert et al., Cell 172:650-665, 2018; Thurman et al., Nature 489:75-82, 2012; Weirauch et al., Cell 158:1431-1443, 2014) (FIGS. 25a and 26a). As iDAPT enables profiling of both genomic and proteomic content of the open chromatin landscape, we sought to compare transcription factor enrichment profiles obtained from iDAPT-MS and iDAPT-seq approaches. To assess the enrichment of transcription factors obtained via iDAPT-seq, we profiled both nuclei and “naked” genomic DNA from both K562 and NB4 cell lines. iDAPT-seq analysis confirms loss of both nucleosomal enrichment and promoter insertion preference in naked DNA. Furthermore, insertion profiles segregate along the first principal component and exhibit skewed statistical significance towards chromatinized peaks in both datasets (FIGS. 26b-26h).

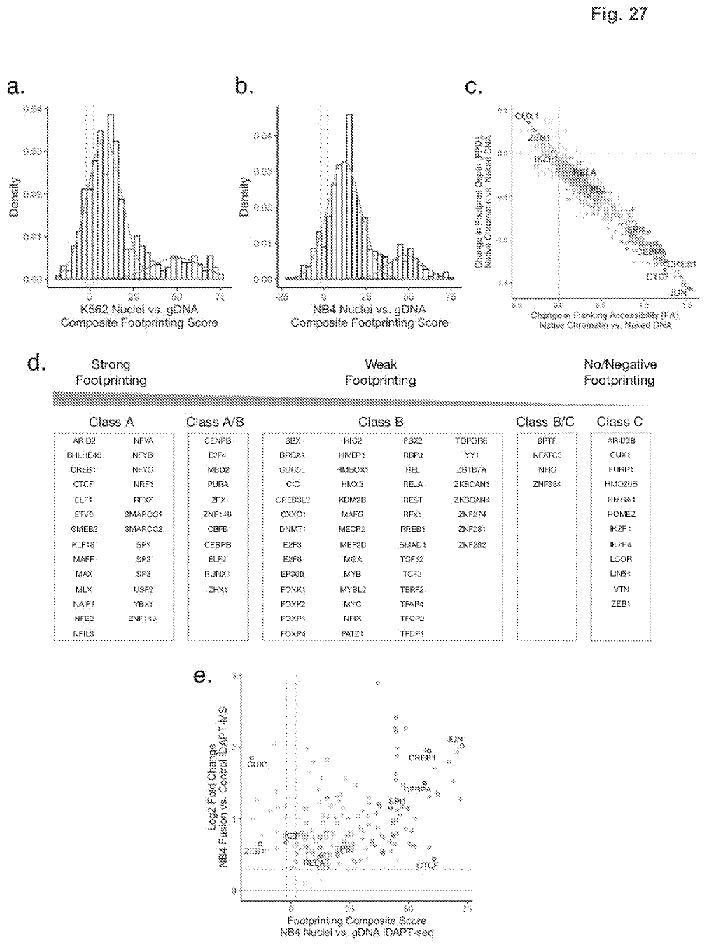

With these iDAPT-seq profiles, we performed footprinting analysis to infer transcription factor activities at their cognate motifs. By a genome-wide bivariate footprinting approach, accounting for both transcription factor footprint depth (FPD) and flanking chromatin accessibility (FA) near the transcription factor motif, we observed significant enrichment of most CisBP transcription factor motifs in iDAPT-seq profiles from native chromatin (Baek et al., Cell Rep. 19:1710-1722, 2017; Weirauch et al., Cell 158:1431-1443, 2014) (FIGS. 25b, 25c, and 27a-27c). We categorized motifs emerging from our footprint analysis into three classes: strong footprinting (class A), weak footprinting (class B), and no or negative footprinting (class C) (FIG. 27d). In line with previous reports, transcription factors with longer residence times on chromatin exhibit stronger footprints: for instance, CTCF, an insulator protein with a long retention time on DNA, exhibits a strong footprint (class A) and is detected by both iDAPT-MS and ChIP-seq (Sung et al., Nat. Methods 13:222-228, 2016; Nakahashi et al., Cell Rep. 3:1678-1689, 2013) (FIG. 25d). RELA/NF-κB complexes (class B) have short DNA residence times and substantially weaker footprinting potential, despite being detected by both iDAPT-MS and ChIP-seq (Bosisio et al., EMBO J. 25:798-810, 2006) (FIG. 25e). While class C motifs such as IKZF1 exhibit nonsignificant or even significantly negative footprinting activity, several of these transcription factors are nonetheless found on open chromatin by both iDAPT-MS and ChIP-seq (FIGS. 25f-25h). Broadly, we observed no clear relationship between inferred transcription factor footprint activity by iDAPT-seq and magnitude of transcription factor abundance by iDAPT-MS (FIGS. 25g and 27e). Indeed, ChIP-seq and iDAPT-MS both directly identify transcription factors spanning all three classes of footprint activities (FIG. 25h), yet neither assay alone can inform how transcription factor binding might affect chromatin accessibility. Conversely, footprinting analysis of iDAPT-seq is able to detect changes to chromatin accessibility, but these changes may be independent of whether a transcription factor is bound or not. Thus, we posit that, for the analysis of transcription factors with annotated motifs, iDAPT-seq and iDAPT-MS together identify transcription factors bound to open chromatin and reveal their activity on chromatin accessibility as a consequence of their abundance, providing greater insight into transcription factor mechanisms than either assay alone.

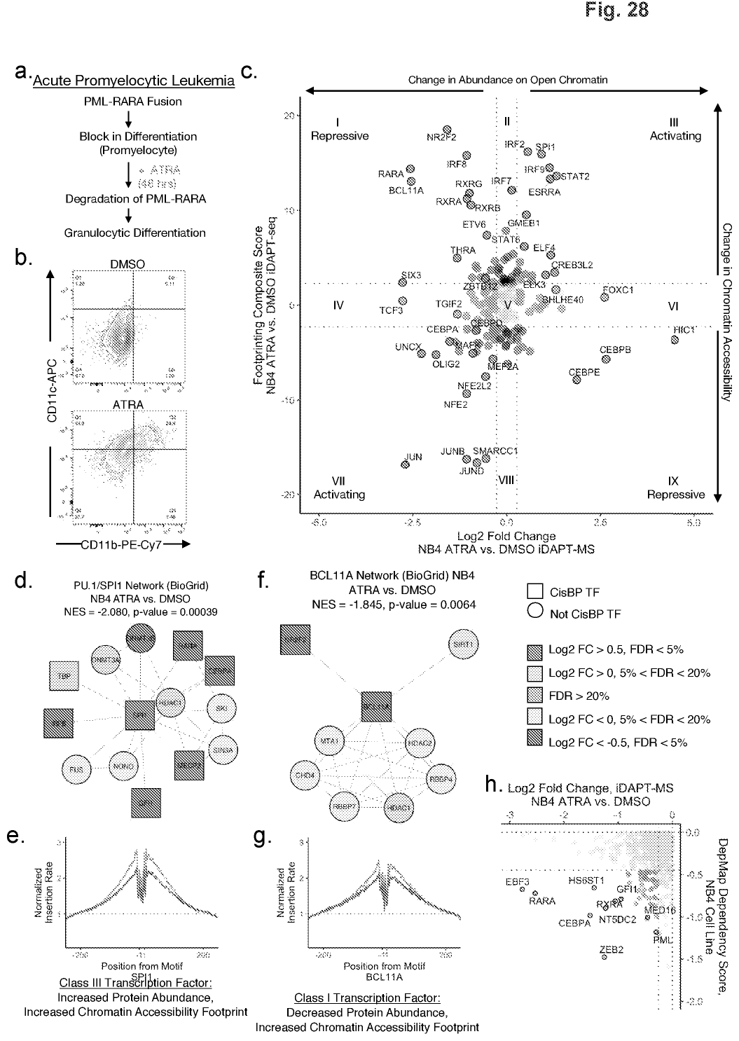

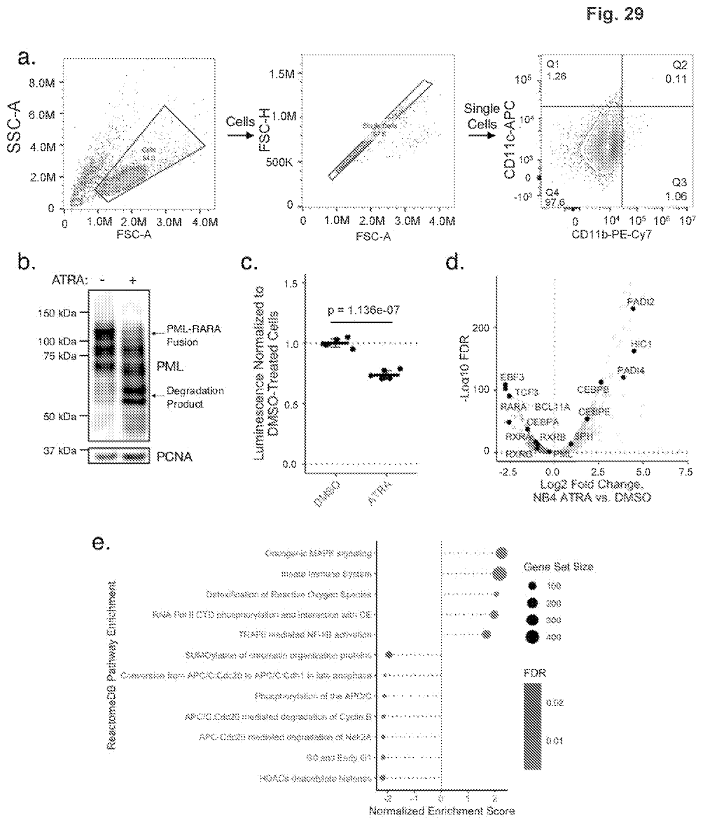

We assessed how transcription factor abundances and chromatin accessibility states correlate upon granulocytic differentiation of the NB4 acute promyelocytic leukemia (APL) cell line. Differentiation of NB4 cells via all-trans retinoic acid (ATRA) leads to degradation of the PML-RARA oncogenic fusion protein, decreased proliferation, and granulocytic differentiation of the leukemia (Lanotte et al., Blood 77:1080-1086, 1991) (FIGS. 28a, 28b, and 29a-29c). iDAPT-MS reveals a dramatic shift in the open chromatin proteome, with profiles clustering by treatment (FIGS. 20b and 20d). In line with previous reports, we observed negative enrichment of RARA, degraded upon ATRA treatment (Zhu et al., PNAS USA 96:14807-14812, 1999; de The et al., Cell 66:675-684, 1991), and positive enrichment of PU.1/SPI1, CEBPB, and CEBPE, upregulated in response to ATRA (Mueller et al., Blood 107:3330-3338, 2006; Chih et al., Blood 90:2987-2994, 1997) (FIG. 29d). Pathway enrichment analysis reveals positive associations with MAPK signaling, neutrophil differentiation, and the innate immune response (FIG. 29e). On the other hand, loss of histone deacetylase enrichment, the most significantly negative pathway, may explain the previously described decrease in histone acetylation states and sensitivity to histone deacetylase inhibitors in APL (Martens et al., Cancer Cell 17:173-185, 2010; Warrell et al., J. Natl. Cancer Inst. 90:1621-1625, 1998). These observations validate the ability of iDAPT-MS to capture both specific proteins and proteomic signatures as they dynamically shift upon changes in cell identity.

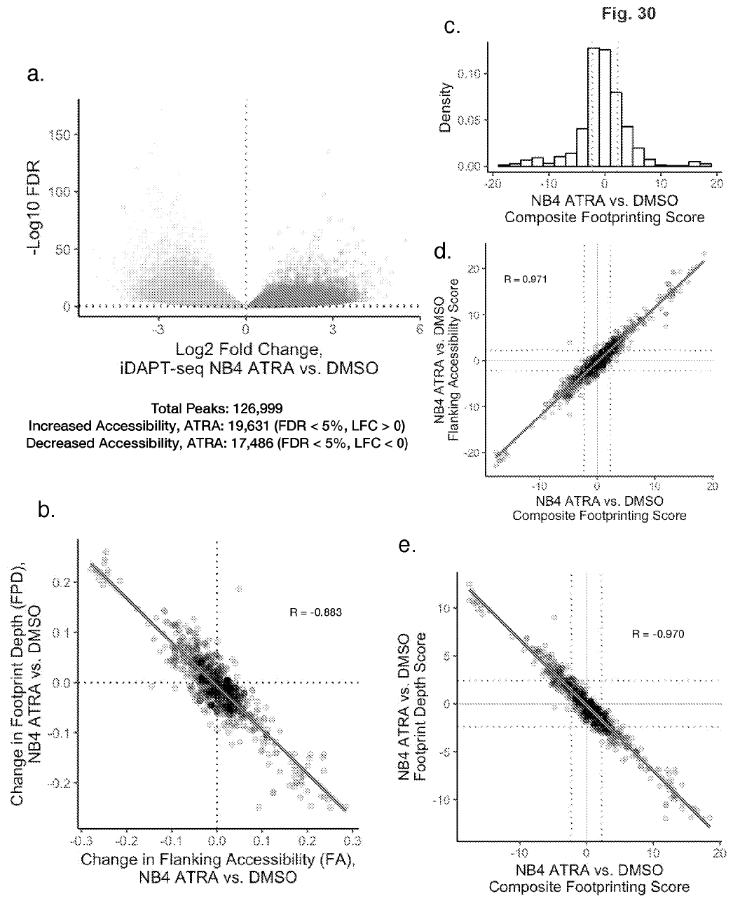

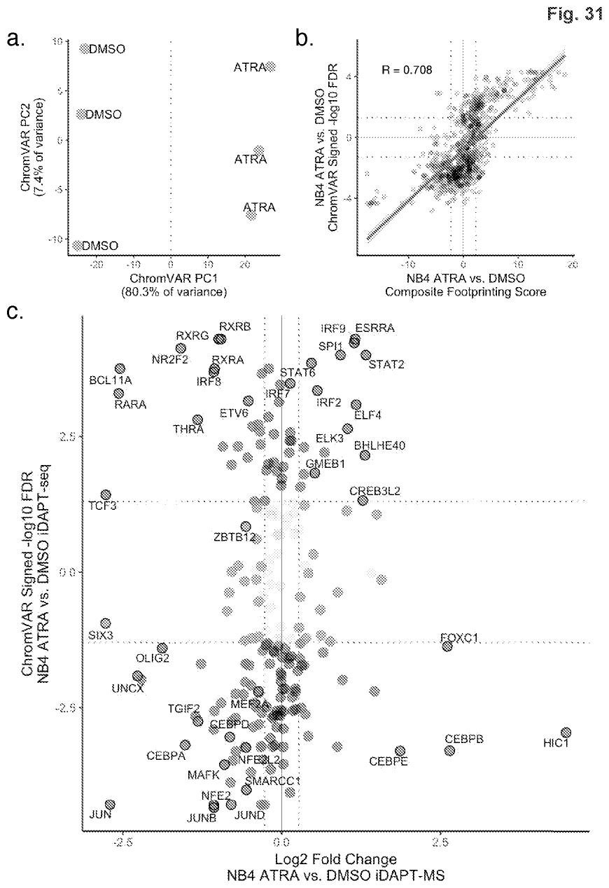

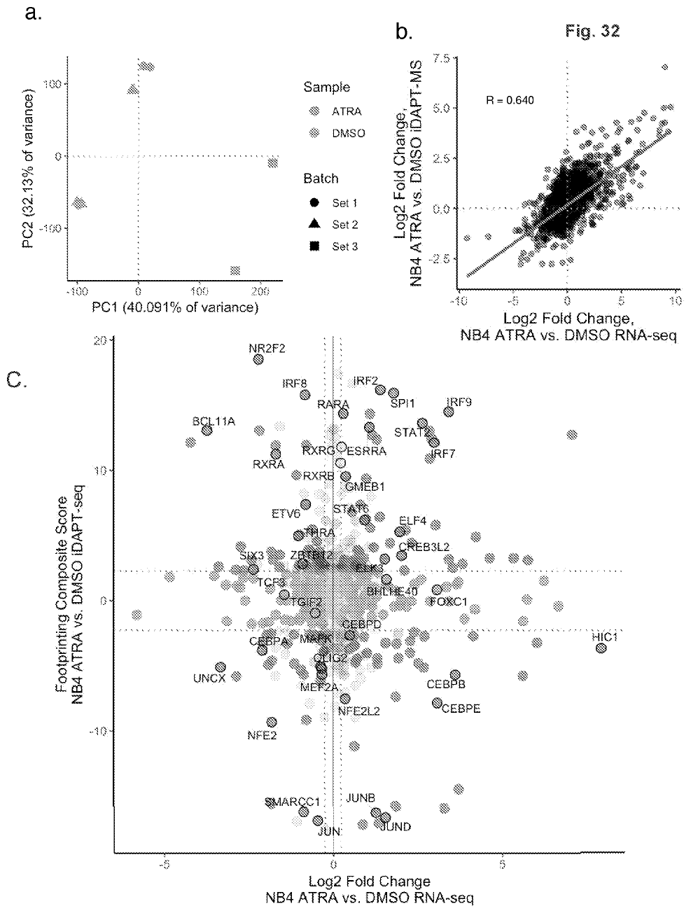

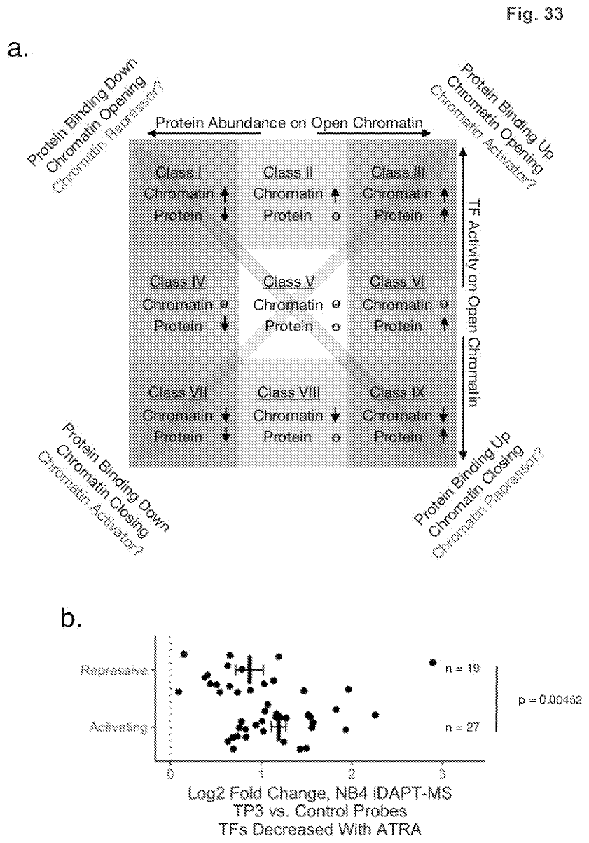

Given the different transcription factor classes captured by iDAPT at steady state, we explored how transcription factor activities and abundances change on open chromatin upon ATRA-mediated cellular differentiation. By iDAPT-seq, we observed both increased and decreased regions of open chromatin and motif footprinting activity upon ATRA treatment, with footprinting parameters FPD and FA correlating strongly with composite footprinting scores (FIG. 30). Intriguingly, both concordant and discordant enrichment patterns between iDAPT-seq and iDAPT-MS transcription factor enrichment profiles were observed (FIG. 28c). Furthermore, some transcription factors exhibit only one of either differential footprinting or protein abundance, discrepancies that have been observed previously between chromatin accessibility and chromatin immunoprecipitation-based assays (Sung et al., Nat. Methods 13:222-228, 2016; Baek et al., Cell Rep. 19:1710-1722, 2017) (FIG. 28c). To corroborate our findings, we replaced our iDAPT-seq footprinting and iDAPT-MS analyses with either motif enrichment analysis via ChromVAR or RNA-seq analysis, which correlates well with our iDAPT-MS protein analysis, both yielding similar transcription factor patterns (Schep et al., Nat. Methods 14:975-978, 2017; Witzel et al., Nat. Genet. 49:742-752, 2017; Orfali et al., Eur. J. Haematol. 104:236-250, 2020) (FIGS. 31 and 32). Hence, iDAPT reveals nine distinct classes (classes I-IX) arising as a consequence of integrating both iDAPT-seq, a readout of transcription factor activity, and iDAPT-MS, a readout of transcription factor protein abundance at open chromatin (FIGS. 28c and 33a). Furthermore, we interpreted concordance (classes III, VII) as chromatin activating activity by the transcription factor of interest and discordance (classes I, IX) as chromatin repression (FIGS. 28c and 33a). In support of this functional classification scheme, among transcription factors decreasing in abundance upon ATRA treatment, those classified as activating (class VII), which should be easier to tag by TP fusion proteins in the vehicle-treated setting, are generally more enriched by TP3 over negative control probes than repressive transcription factors (class I) (FIG. 33b). Thus, iDAPT-MS and iDAPT-seq together uncover functional relationships between transcription factor binding dynamics and chromatin accessibility, which neither assay can elucidate alone.

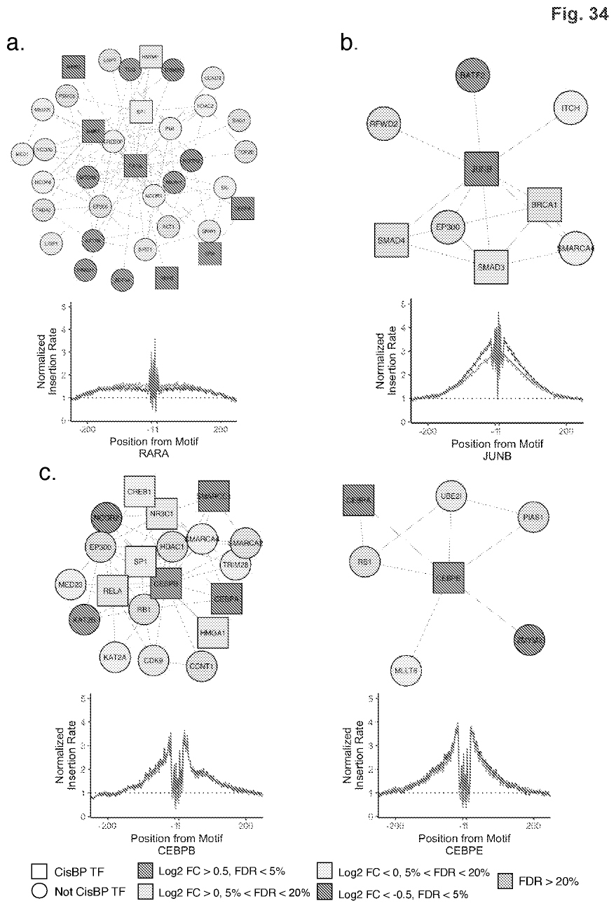

As iDAPT-MS reveals abundance changes of proteins beyond transcription factors, we assessed how proteins interacting with transcription factors may cooperate to regulate chromatin accessibility states. For a given transcription factor, we superimposed iDAPT-MS protein abundance changes onto its first-order protein interaction network from BioGrid (Oughtred et al., Nuc. Acids Res. 47:D529-D541, 2019). Of these putative transcription factor complex profiles, we found the PU.1/SPI1 protein interaction network to be the most significantly decreased complex upon ATRA treatment (FIG. 28d). Intriguingly, while many of its protein interactors such as the transcriptional corepressor SIN3A decrease in abundance, PU.1/SPI1 itself increases in abundance to promote chromatin accessibility at its cognate motif (class III) (Mueller et al., Blood 107:3330-3338, 2006; Hu et al., Blood 117:6498-6508, 2011) (FIGS. 28d and 28e). Furthermore, the decrease in RARA protein abundance, also an interactor of PU.1/SP11, leads to increased chromatin accessibility at its binding motif due to its ATRA-mediated degradation, implicating its transcriptional repressive activity (class I) (Wang et al., Cancer Cell 17:186-197, 2010) (FIG. 34a). Thus, in the APL setting, transcriptional repressors bind to PU.1/SPI1 to repress chromatin accessibility at PU.1/SPI1 motifs; this repressive binding is relieved upon ATRA treatment, enabling PU.1/SPI1 to activate transcription at its motifs. This analysis may be extended to other transcription factors and their protein complexes: BCL11A, together with many of its annotated protein interactors, decreases in abundance while increasing chromatin accessibility upon ATRA treatment (class I), suggestive of a coordinated downregulation of this repressive transcription factor and its protein complex components (Liu et al., Cell 173:430-442.e17, 2018) (FIGS. 28f and 28g). While JUNB (Li et al., EMBO J. 18:420-432, 1999; Schutte et al., Cell 59:987-997, 1989; Chiu et al., Cell 59:979-986, 1989), CEBPB (Descombes et al., Cell 67:569-579, 1991), and CEBPE (Bedi et al., Blood 113:317-327, 2009) have both activating and repressive behaviors reported, we observed class VII activating behavior from the JUNB transcription factor and class IX repressive behavior from the CEBPB and CEBPE transcription factors upon ATRA treatment, with their dynamic protein complex components providing potential context-specific insights into their regulatory activities on chromatin state (FIGS. 34a-34c). In this manner, integrating protein interaction information with iDAPT-MS and iDAPT-seq profiles reveals the interplay between transcription factors, their activities on chromatin accessibility, and their putative protein complexes as these components change during ATRA treatment of NB4 cells.

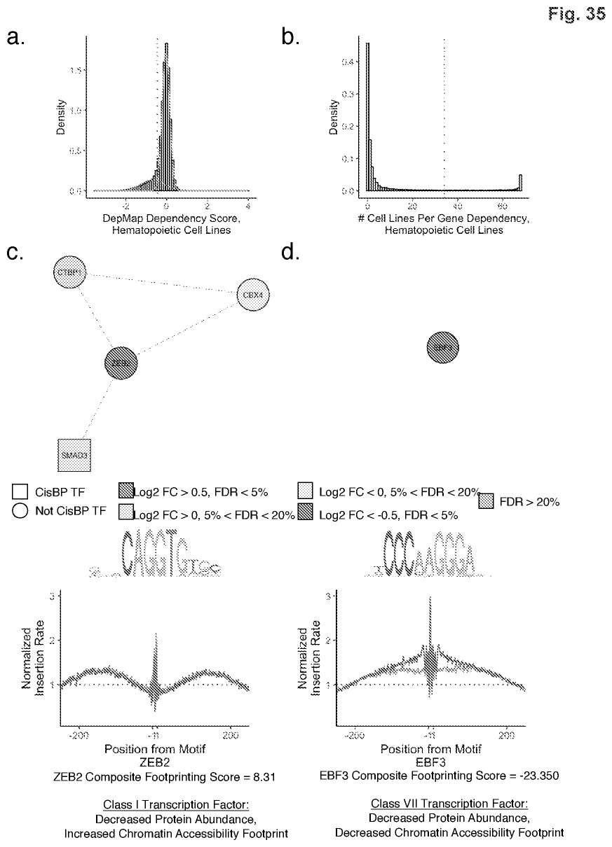

Given the numerous transcription factors and associated components differentially bound at open chromatin upon ATRA treatment, some of these newly identified proteins may have functional roles in APL differentiation. We superimposed our iDAPT-MS results with NB4 genetic dependencies and identified both PML and RARA, corroborating our analysis (Meyers et al., Nat. Genet. 49:1779-1784, 2017) (FIG. 28h). After filtering out essential genes across hematopoietic cell lines, we identified a number of candidate transcription factor effectors, including CEBPA, EBF3, and ZEB2, which may act downstream or independently of PML-RARA (FIGS. 28h and 35). In agreement with previous reports, our transcription factor classification scheme assigns ZEB2 as repressive (Postigo et al., PNAS USA 97:6391-6396, 2000) (class I) and EBF3 (Sleven et al., Am. J. Hum. Genet. 100:138-150, 2017; Chao et al., Am. J. Hum. Genet. 100:128-137, 2017; Harms et al., Am. J. Hum. Genet. 100:117-127, 2017) and CEBPA (Pabst et al., Nat. Genet. 27:263-270, 2001) as activating (class VII) (FIGS. 28c, 35c, and 35d). This analysis reifies the power of combining forward genetic screens with iDAPT-MS to identify critical transcription factors and their regulators for a given biological phenotype.

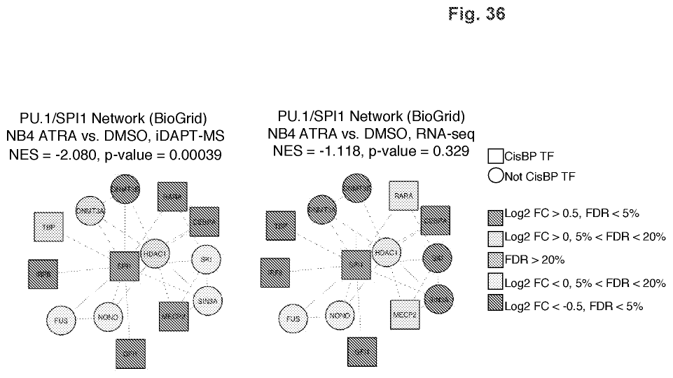

Finally, we assessed how our interpretations of transcription factor dynamics would change between iDAPT-MS, measuring protein abundances directly, and RNA-seq profiles. While we observed a positive correlation between iDAPT-MS and RNA-seq profiles upon ATRA treatment, several discordant cases emerged, including JUNB/JUND and RARA, with their RNA-seq effect sizes opposite in magnitude of their corresponding iDAPT-MS effects (FIGS. 28c, 32b, and 32c). Indeed, ATRA binds to RARA, and prolonged ligand binding and transcriptional activity leads to RARA protein degradation (Zhu et al., PNAS USA 96:14807-14812, 1999) (FIG. 34a). Furthermore, as transcript levels of RARA and several other protein interactors of PU.1/SPI1 do not fully match iDAPT-MS enrichment trends, the significantly negative enrichment of the PU.1/SPI1 protein complex observed upon ATRA treatment by iDAPT-MS is lost by RNA-seq (FIG. 36). Thus, among open chromatin-associated proteins, bulk RNA-seq may broadly provide similar patterns as iDAPT-MS, but discrepancies between the two limit the ability of RNA-seq to replace proteomic analysis.

### Methods

Cell lines and culture conditions. HT1080 (American Type Culture Collection, ATCC) were cultured in EMEM (ATCC) supplemented with 10% FBS and 1% penicillin/streptomycin. K562 (ATCC) cells were cultured in RPMI-1640 supplemented with 10% FBS and 1% penicillin/streptomycin. NB4 cells (DSMZ) were cultured in RPMI-1640 supplemented with 10% charcoal-stripped FBS (Gibco) and 1% penicillin/streptomycin. All-trans retinoic acid (ATRA, Sigma) was dissolved in DMSO at a concentration of 10 mM. Cells were incubated at 37° C. and 5% CO₂. Genomic DNA was extracted from K562 and NB4 cells using the Quick-DNA MiniPrep kit (Zymo).

Cloning and purification of recombinant proteins, and transposome adaptor preparation. Cloning and purification of recombinant proteins is as described in Example 1, above. Plasmids containing C-terminally tagged gene constructs as described in this study are deposited to Addgene (#160081, #160083-160088). Transposome adaptor preparation is as described in Example 1, above.

ATAC-seq/iDAPT-seq sample preparation. The OmniATAC sample preparation protocol was used as previously described (Corces et al., Nat. Methods 14:959-962, 2017) with modifications where indicated below. 10 pmol enzyme (2 μL in 2×DB) was mixed with 12.5 pmol MEDS-A/B (1.25 μL in water) and incubated at room temperature for 1 hour. In the meantime, 50,000 cells were centrifuged at 500×g for 5 minutes at 4° C. Cells were resuspended in 50 μL lysis buffer 1 (LB1: 10 mM Tris-HCl pH 7.5, 10 mM NaCl, 3 mM MgCl2, 0.01% digitonin, 0.1% Tween-20, and 0.1% NP-40) with trituration, incubated on ice for 3 minutes, and then further supplemented with 1 mL lysis buffer 2 (LB2: 10 mM Tris-HCl pH 7.5, 10 mM NaCl, 3 mM MgCl2, and 0.1% Tween-20). Nuclei were pelleted (500×g, 10 minutes, 4° C.), resuspended with 50 μL tagmentation reaction mixture (20% dimethylformamide, 10 mM MgCl2, 20 mM Tris-HCl pH 7.5, 33% 1×PBS, 0.01% digitonin, 0.1% Tween-20, and either 10 pmol enzyme equivalent of enzyme:DNA complex or 2.5 μL Nextera Tn5 [Illumina, TDE1 from FC-121-1030] in 50 μL total volume), and incubated at 37° C. for 30 minutes with agitation on a thermomixer (1,000 rpm). For iDAPT-seq libraries generated from K562 or NB4 cells or genomic DNA, bovine serum albumin (BSA) was added at a final concentration of 1% to lysis (LB1 and LB2) and tagmentation buffers. Tagmentation with naked genomic DNA was performed using 50 ng genomic DNA as substrate. After tagmentation, DNA libraries were extracted with DNA Clean and Concentrator-5 (Zymo) and eluted with 21 μL water.

To determine optimal PCR cycle number for library amplification, quantitative PCR was performed on a StepOnePlus Real-Time PCR (Applied Biosystems) with the StepOne v2.3 software (Buenrostro et al., Nat. Methods 10:1213-1218, 2013). 2 μL of each ATAC-seq or iDAPT-seq library was added to 2× NEBNext Master Mix (NEB) and 0.4×SYBR Green (Thermo Fisher) with 1.25 μM of each primer (Primer 1: 5′-AATGATACGGCGACCACCGAGATCTACACTCGTCGGCAGCGTCAGATGTG-3′; Primer 2.1: 5′-CAAGCAGAAGACGGCATACGAGATTCGCCTTAGTCT CGTGGGCTCGGAGATGT-3′) in a final volume of 15 μL, and quantification was assessed using the following conditions: 72° C. for 5 minutes; 98° C. for 30 seconds; and thermocycling at 98° C. for 10 seconds, 63° C. for 30 seconds and 72° C. for 1 minute. Optimal PCR cycle number was determined as the qPCR cycle yielding fluorescence between 1/4 and 1/3 of the maximum fluorescence. The remaining DNA library was then amplified accordingly by PCR using previously reported barcoded primers for library multiplexing (Buenrostro et al., Nat. Methods 10:1213-1218, 2013), purified with DNA Clean and Concentrator-5 (Zymo), and eluted into 20 μL final volume with water. Libraries were then subject to TapeStation 2200 High Sensitivity D1000 or D5000 fragment size analysis (Agilent) and NextSeq 500 High Output paired-end sequencing (2×75 bp, Illumina) as indicated.

ATAC-seq/iDAPT-seq data preprocessing. Paired-end sequencing reads were trimmed with TrimGalore v0.4.5 to remove adaptor sequence CTGTCTCTTATACACATCT (SEQ ID NO: 35), which arises at the 3′ end due to sequenced DNA fragments being shorter than the sequencing length (75 bp). Reads were aligned to the hg38 reference genome using bowtie2 v2.2.9 with options “--no-unal--no-discordant--no-mixed-X 2000”. Reads mapping to the mitochondrial genome were subsequently removed, and duplicate reads were removed with Picard v2.8.0. For insert size distribution, transcription start site (TSS) enrichment, and genome track visualization analyses, reads were downsampled to approximately 5 million paired-end fragments. Insert size distributions were determined by counting inferred fragment sizes from read alignments. TSS enrichment was performed by first shifting insert positions aligned to the reverse strand by −5 bp and the forward strand by +4 bp as previously described (Buenrostro et al., Nat. Methods 10:1213-1218, 2013) and then determining the distance of each insertion to the closest Ensembl v94 transcription start site with Homer v4.9. Visualization was performed by mapping insertions to a genome-wide sliding 150 bp window with 20 bp offsets with bedops v2.4.30, followed by conversion to bigwig format with wigToBigWig from UCSC tools v363. Genome tracks were visualized with Integrative Genomics Viewer v2.5.0.

Peaks were aligned by MACS2 v2.1.1 using options “callpeak--nomodel--shift-100--extsize 200--nolambda-q 0.01--keep-dup all”, generating either individual peak sets from each library (GM12878 analysis) or a consensus peak set after consolidating all reads (K562, NB4 analyses). For GM12878 analysis, a union of all analyzed peaks was taken as a consensus peak set and counts of insertions within peaks (downsampled to 5 million reads) were assessed using bedtools v2.26.0 with the multicov function. Correlation analysis was performed with log 2 read counts +1 and visualized using the pheatmap function in R v3.5.0. For K562 and NB4 analyses, consensus peaks overlapping with hg38 blacklist regions were removed (https://www.encodeproject.org/annotations/ENCSR636HFF/) and counts of insertions within peaks were assessed using the bedtools multicov function. Count matrices were processed with DESeq2 for differential insertions with shrunken log 2 fold changes, and principal component analyses were performed with counts transformed by the varianceStabilizingTransformation function from DESeq2. Figures were generated with ggplot2 v3.1.1.

Co-immunofluorescence/ATAC-see analysis. ATAC-see was performed similarly as previously described with slight modifications (Chen et al., Nat. Methods 13:1013-1020, 2016). Enzyme and transposon DNA were mixed at a 1:1.25 enzyme:MEDS-A/B-AF647 molar ratio and incubated at room temperature for 1 hour. Adherent cells were grown on glass coverslips (Fisher Scientific, 12-540A) until 80-90% confluent, washed with 1×PBS, fixed with 1% formaldehyde (Electron Microscopy Services) in 1×PBS for 10 minutes, and washed twice with ice-cold 1×PBS. Immobilized cells were lysed by incubation with LB1 for 3 minutes followed by LB2 for 10 minutes at room temperature. Cells were then subject to tagmentation (20% dimethylformamide, 10 mM MgCl2, 20 mM Tris-HCl pH 7.5, 33% 1×PBS, 0.01% digitonin, 0.1% Tween-20, and 80 pmol enzyme equivalent of enzyme:DNA complex in a total volume of 100 μL) for 30 minutes at 37° C. in a humidified chamber. Subsequently, cells were washed with 50 mM EDTA and 0.01% SDS in 1×PBS three times for 15 minute each at 55° C., lysed for 10 minutes with 0.5% Triton X-100 in 1×PBS at room temperature, and blocked with 1% BSA and 10% goat serum in PBS-T (1×PBS and 0.1% Tween-20) for 1 hour in a humidified chamber. Primary antibody was added to slides in 1% BSA/PBS-T and incubated at 4° C. overnight; slides were then washed and subjected to secondary antibody staining for 1 hour. Slides were washed with PBS-T three times for 15 minutes each, stained with DAPI (Sigma, 1 μg/mL) for 1 minute, washed with PBS for 10 minutes, and mounted with Fluorescence Mounting Medium (Dako). Confocal microscopy images were taken with an LSM 880 Axio Imager 2 or an LSM 880 Axio Observer at 63× magnification (Zeiss). Images were processed with Fiji/ImageJ v2.0.0.

Primary antibodies used were anti-RNA polymerase II CTD repeat YSPTSPS (phospho S2) (rabbit, Abcam ab5095, 1:500), anti-H3K27Ac (rabbit, Abcam ab4729, 1:500), anti-H3K9me3 (rabbit, Abcam ab8898, 1:500), anti-SC35 (mouse, SC-35, Abcam ab11826, 1:1000). Secondary antibodies used were Goat anti-Rabbit IgG (H+L) Secondary Antibody, Alexa Fluor 488 conjugate (Thermo Fisher Scientific A11008, 1:1000) and Goat anti-Mouse IgG (H+L) Cross-Adsorbed Secondary Antibody, Alexa Fluor 488 conjugate (Thermo Fisher Scientific A11001, 1:1000).

Quantitative image analyses were performed with CellProfiler v3.1.5. Region of interests (ROIs) were identified from DAPI channel intensity values using minimum cross entropy thresholding, with each ROI corresponding to an individual nucleus. Pearson correlation coefficients were determined by comparing ATAC-see pixel intensities with corresponding immunofluorescence intensity values within each ROI to assess the nucleus-to-nucleus variation in colocalization.

Peroxidase activity assay. 5 pmol enzyme was incubated with 2.5 pmol hemin chloride (Cayman Chemical, dissolved in DMSO) for 1 hour at room temperature. This molar ratio was selected given reports of APEX2 maximal heme occupancy between 40-57%. Heme:protein complexes were then subjected to 50 μM Amplex UltraRed (Thermo Fisher Scientific) and 1 mM hydrogen peroxide for 1 minute at room temperature in a total volume of 100 μL with 1×PBS. Reactions were then quenched with 100 μL 2× quenching solution (10 mM Trolox, 20 mM sodium ascorbate, and 20 mM NaN₃ in 1×PBS), and fluorescence intensities were measured on a SpectraMax iD3 plate reader with the SoftMax Pro v7.0.3 software, with excitation at 530 nm and emission at 590 nm.

DNA and protein tagging by iDAPT. All iDAPT proteomic labeling assays were performed as described below unless indicated otherwise. 2.5 μmol MEDS-A/B, 2 μmol enzyme, and 1 μmol hemin chloride per channel were incubated at room temperature for 1 hour. 1e7 cells per sample were washed (500×g, 5 minutes, 4° C.), lysed and triturated in 100 μL LB1 (10 mM Tris-HCl pH 7.5, 10 mM NaCl, 3 mM MgCl2, 1% BSA, 0.01% digitonin, 0.1% Tween-20, 0.1% NP-40, and 1× cOmplete EDTA-free protease inhibitor cocktail [Roche]) for 3 minutes, and subsequently supplemented with an additional 1 mL of LB2 (10 mM Tris-HCl pH 7.5, 10 mM NaCl, 3 mM MgCl2, 1% BSA, 0.1% Tween-20, and 1× protease inhibitor). Nuclei were pelleted (500×g, 10 minutes, 4° C.), resuspended with tagmentation reaction mixture (20% dimethylformamide, 10 mM MgCl2, 20 mM Tris-HCl pH 7.5, 33% 1×PBS, 1% BSA, 0.01% digitonin, 0.1% Tween-20, 500 μM biotin-phenol, 1× protease inhibitor, and 2 pmol enzyme equivalent of enzyme:DNA:heme complex in a total volume of 500 μL), and incubated at 37° C. for 30 minutes with agitation on a thermomixer (1,000 rpm). 5 μL of tagmentation mix was saved for quality assessment as described above for ATAC-seq/iDAPT-seq sample preparation. The remaining nuclear suspension was then washed 2× with 1×PBS supplemented with 500 μM biotin-phenol, 1% BSA, 0.1% Tween-20, and 1× protease inhibitor (3000×g, 5 minutes, 4° C.) and labeled with 1 mM hydrogen peroxide and 500 μM biotin-phenol for 1 minute in 1×PBS with 1× protease inhibitor in a volume of 500 μL. Peroxidation reactions were quenched with 500 μL 2× quenching buffer (10 mM Trolox, 20 mM sodium ascorbate, 20 mM NaN₃, and 1× protease inhibitor in 1×PBS). Labeled nuclei were then pelleted, washed with 1× quenching buffer, resuspended in 500 μL RIPA containing protease inhibitors, and frozen at −80° C. Lysates were thawed on ice, sonicated via a Sonic Dismembrator 100 (Fisher Scientific, setting 3, 15 seconds, 4 pulses), and incubated on ice for 30 minutes after the addition of 1 μL benzonase (EMD Millipore). Lysates were clarified by centrifugation (15,000×g, 20 minutes, 4° C.), quantified via the detergent-compatible Bradford assay (Thermo Fisher Scientific), and subjected to either Western blotting or quantitative mass spectrometry analyses as described below. For NB4 cell analysis, an additional endogenous peroxidase blocking step was added after nuclear extraction and before tagmentation: nuclei were resuspended in 500 μL 1×PBS containing 1% BSA, 0.03% hydrogen peroxide, and 0.1% NaN₃ and incubated on ice for 30 minutes. Nuclei were pelleted and washed 4× with 1×PBS/1% BSA (3000×g, 5 minutes, 4° C.). Residual hydrogen peroxide was monitored by colorimetric assessment of supernatant via Quantofix peroxides test stick (Sigma).

Western blotting analysis. Whole cell or nuclear lysates were generated by resuspending cells or nuclei in RIPA (Boston BioProducts) supplemented with 1× cOmplete EDTA-free protease inhibitor cocktail (Roche). Lysates were incubated on ice for 30 minutes, sonicated via a Sonic Dismembrator 100 (Fisher Scientific) at setting 3 with 3-4 pulses of 15 seconds on/off on ice, and treated with benzonase for an additional 30 minutes on ice. Lysates were clarified by centrifugation (15,000×g, 20 minutes, 4° C.) and their concentrations quantified via the detergent-compatible Bradford assay (Thermo Fisher Scientific). All Western blots were run on NuPAGE 4-12% Bis-Tris protein gels (Thermo Fisher Scientific) and transferred to 0.2 μm nitrocellulose membranes (GE Healthcare). Membranes were blocked with 3% milk in PBS-T and incubated overnight with primary antibody and subsequently with secondary antibody after brief washing with PBS-T. Chemiluminescence was determined by applying ECL Western Blotting detection reagent (GE Healthcare) to membranes and imaging on an Amersham Imager 600 (GE Healthcare). Membranes were stripped with Restore PLUS Stripping Buffer (Thermo Fisher Scientific).

Primary antibodies used were anti-FLAG M2 (mouse, Sigma-Aldrich, F1804, 1:2000), anti-PCNA (mouse, PC10, Santa Cruz Biotechnology sc-56, 1:1000), and anti-PML (rabbit, Bethyl A301-167A, 1:1000). Secondary antibodies used were Rabbit IgG, HRP-linked F(a13)₂ fragment (GE Healthcare NA9340, from donkey, 1:5000) and Mouse IgG, HRP-linked whole Ab (GE Healthcare NA931, from sheep, 1:5000). Streptavidin-HRP (Cell Signaling Technology #3999S, 1:1000) was also used for probing.

Streptavidin enrichment and tandem mass tag labeling. 250 μg (K562) or 150 μg (NB4) lysate was reduced with 5 mM DTT and then added to 60 μL (K562) or 90 μL (NB4) Pierce streptavidin bead slurry equilibrated 2× with RIPA buffer. Lysate/bead mixture was incubated with end-to-end rotation overnight at 4° C. Beads were washed 3× with RIPA, 2× with 200 mM EPPS pH 8.5, and resuspended with 100 μL 200 mM EPPS pH 8.5, with beads resuspended and incubated with end-to-end rotation for 5 minutes per wash. 1 μL mass spectrometry-grade LysC (Wako) was added to each tube and incubated at 37° C. for 3 hours with mixing, and an additional 1 μL mass spectrometry-grade trypsin (Thermo Fisher Scientific) was added, followed by overnight incubation at 37° C. with mixing. Beads were magnetized, and eluate was collected and subjected to downstream TMT labeling.

Peptides were processed using the SL-TMT method (Navarrete-Perea et al., J. Proteome Res. 17:2226-2236, 2018). TMT reagents (0.8 mg) were dissolved in anhydrous acetonitrile (40 μL), of which 10 μL was added to each peptide suspension (100 μL) with 30 μL of acetonitrile to achieve a final acetonitrile concentration of approximately 30% (v/v). Following incubation at room temperature for 1 hour, the reaction was quenched with hydroxylamine to a final concentration of 0.3% (v/v). The TMT-labeled samples were pooled at a 1:1 ratio across all samples. The pooled sample was vacuum centrifuged to near dryness and subjected to C18 solid-phase extraction (SPE) (Sep-Pak, Waters).

Off-line basic pH reversed-phase (BPRP) fractionation. We fractionated the pooled TMT-labeled peptide sample using BPRP HPLC (Wang et al., Proteomics 11:2019-2026, 2011). We used an Agilent 1200 pump equipped with a degasser and a photodiode array (PDA) detector (set at 220 and 280 nm wavelength) from ThermoFisher Scientific (Waltham, Mass.). Peptides were subjected to a 50-minute linear gradient from 9% to 35% acetonitrile in 10 mM ammonium bicarbonate pH 8 at a flow rate 600 μL/min over an Agilent 300Extend C18 column (3.5 pm particles, 4.6 mm ID and 220 mm in length). The peptide mixture was fractionated into a total of 96 fractions, which were consolidated into 24 super-fractions (Paulo et al., J. Proteomics 148:85-93, 2016). Samples were subsequently acidified with 1% formic acid and vacuum centrifuged to near dryness. Each consolidated fraction was desalted via StageTip, dried again via vacuum centrifugation, and reconstituted in 5% acetonitrile, 5% formic acid for LC-MS/MS processing.

LC-MS/MS proteomic analysis. Samples were analyzed on an Orbitrap Fusion mass spectrometer (Thermo Fisher Scientific, San Jose, Calif.) coupled to a Proxeon EASY-nLC 1200 liquid chromatography (LC) pump (Thermo Fisher Scientific). Peptides were separated on a 100 pm inner diameter microcapillary column packed with 35 cm of Accucore C18 resin (2.6 pm, 150 Å, ThermoFisher). For each analysis, approximately 2 μg of peptides were separated using a 150 min gradient of 8 to 28% acetonitrile in 0.125% formic acid at a flow rate of 450-500 nL/minute. Each analysis used an MS3-based TMT method (Ting et al., Nat. Methods 8:937-940, 2011; McAlister et al., Anal. Chem. 86:7150-7158, 2014), which has been shown to reduce ion interference compared to MS2 quantification (Paulo et al., J. Am. Soc. Mass Spectrom. 27:1620-1625, 2016). The scan sequence began with an MS1 spectrum (Orbitrap analysis, resolution 120,000, 350-1400 Th, automatic gain control (AGC) target 2e5, maximum injection time 100 ms). The top ten precursors were then selected for MS2/MS3 analysis. MS2 analysis consisted of: collision-induced dissociation (CID), quadrupole ion trap analysis, automatic gain control (AGC) 1.4e4, NCE (normalized collision energy) 35, q-value 0.25, maximum injection time 120 ms), and isolation window at 0.7. Following acquisition of each MS2 spectrum, we collected an MS3 spectrum in which multiple MS2 fragment ions are captured in the MS3 precursor population using isolation waveforms with multiple frequency notches. MS3 precursors were fragmented by HCD and analyzed using the Orbitrap (NCE 65, AGC 1.5e5, maximum injection time 150 ms, resolution was 50,000 at 400 Th).

Proteomic data analysis. Mass spectra were processed using a Sequest-based pipeline (Huttlin et al., Cell 143:1174-1189, 2010). Spectra were converted to mzXML using a modified version of MSConvert. Database searching included all entries from the human UniProt database. This database was concatenated with one composed of all protein sequences in the reversed order. Searches were performed using a 50-ppm precursor ion tolerance for total protein level analysis. The product ion tolerance was set to 0.9 Da. TMT tags on lysine residues and peptide N termini (+229.163 Da) and carbamidomethylation of cysteine residues (+57.021 Da) were set as static modifications, while oxidation of methionine residues (+15.995 Da) was set as a variable modification.

Peptide-spectrum matches (PSMs) were adjusted to a 1% false discovery rate (FDR) (Elias et al., Methods Mol. Biol. 604:55-71, 2010; Elias et al., Nat. Methods 4:207-214, 2007). PSM filtering was performed using a linear discriminant analysis (LDA), as described previously (Huttlin et al., Cell 143:1174-1189, 2010), while considering the following parameters: XCorr, ΔCn, missed cleavages, peptide length, charge state, and precursor mass accuracy. For TMT-based reporter ion quantitation, we extracted the summed signal-to-noise (S:N) ratio for each TMT channel and found the closest matching centroid to the expected mass of the TMT reporter ion. PSMs with poor quality, MS3 spectra with more than eight TMT reporter ion channels missing, MS3 spectra with TMT reporter summed signal-to-noise of less than 100, missing MS3 spectra, or isolation specificity <0.7 were excluded from quantification (McAlister et al., Anal. Chem. 84:7469-7478, 2012).

PSM intensities were normalized by taking the median intensity of streptavidin and trypsin PSMs per sample as a normalization factor, as these proteins are added to each sample in equal amounts post-enrichment. Normalized PSMs were then log 2-transformed and collapsed to proteins by arithmetic average, with priority given to uniquely mapping peptides. Hierarchical clustering, Pearson correlation, and principal component analyses were performed at the protein level. The limma package in R was used to determine differential protein abundances.

Protein enrichment analyses. Gene set enrichment analyses of iDAPT-MS datasets were performed with the fgsea package (10,000 permutations) in R, using UniProt protein identifications ranked by their log 2 fold changes from limma (Ritchie et al., Nuc. Acids Res. 43:e47, 2015). Gene sets used for analyses: CORUM (v3.0) protein complex annotations (Ruepp et al., Nuc. Acids Res. 36:D646-D650, 2008), Human Protein Atlas (v19) subcellular localization annotations with reliability demarcated as “Enhanced” or “Supported” (Thul et al., Science 80:356, eaa13321, 2017), BioGrid (v3.5.178) multi-validated protein interaction annotations (Oughtred et al., Nuc. Acids Res. 47:D529-D541, 2019),

ReactomeDB (v70) pathway to gene mappings from fgsea via the “reactomePathways” function (Fabregat et al., Nuc. Acids Res. 46:D649-D655, 2018), and CisBP transcription factors from the “human_pwms_v2” dataset curated as in the chromVARmotifs package in R (Weirauch et al., Cell 158:1431-1443, 2014; Schep et al., Nat. Methods 14:975-978, 2017). All gene identities were converted to UniProt prior to analysis via biomaRt in R. Protein interaction networks were visualized with igraph v1.2.4.

Four classes of nuclear proteins were collated: histones, chromatin remodelers, transcription factors, and RNA-binding proteins. Histone UniProt IDs were collated from Histone DB 2.0 (Draizen et al., Database 2016, baw014, 2016) and UniProt with search query “Nucleosome core” (The Uniprot Consortium, Nuc. Acids Res. 47:D506-D515, 2019). Chromatin remodeler proteins were obtained from UniProt IDs associated with “GO:0006338” (“chromatin remodeling”) (The Gene Ontology Consortium, Nuc. Acids Res. 47:D330-D338, 2019) and CORUM protein complex components associated with the five primary chromatin remodelers (Ruepp et al., Nuc. Acids Res. 36:D646-D650, 2008): NuRD, SWI, ISWI, 1N080, SWR1. High-confidence RNA binding proteins were obtained from hRBPome (Ghosh et al., doi:https://doi.org/10.1101/269043), 2018, and transcription factors were obtained from Lambert et al. (Lambert et al., Cell 172:650-665, 2018).

K562 RNA-seq (Encode Consortium, Nature 489:57-74, 2012) (ENCFF664LYH and ENCFF855OAF), whole cell proteome (Nusinow et al., Cell 180:387-402.e16, 2020), and nuclear proteome (Federation et al., Cell Rep. 30:2463-2471.e5, 2020) datasets were downloaded and converted to UniProt IDs. RNA-seq genes were filtered for those with nonzero read counts (transcripts per million) in both replicates (Encode Consortium, Nature 489:57-74, 2012). The whole cell proteomic dataset was filtered by removing peptides with missing quantitations (Nusinow et al., Cell 180:387-402.e16, 2020). The nuclear proteome dataset was preprocessed by removing peptides with multiple UniProt IDs and collating remaining UniProt IDs across all salt extraction conditions (Federation et al., Cell Rep. 30:2463-2471.e5, 2020). For determination of proteins associated with specific extraction conditions, we followed a procedure as reported by Federation et al.: peptide intensities were normalized by total intensities for a given sample, collapsed to protein intensities by arithmetic mean, scaled to maximum intensities of 1, and subjected to k-means clustering analysis using k=8 for clustering (Federation et al., Cell Rep. 30:2463-2471.e5, 2020). Protein annotations from Alajem et al. were converted from mouse to human homologs via biomaRt in R, and gene sets (1000U, 45U, 3U) were compiled taking the sets of protein IDs with scores greater than 95 in either ES or NPC sample types (Alajem et al., Cell Rep. 10:2019-2031, 2015).

Additional publicly available open chromatin proteome datasets were downloaded, and gene identities were converted to UniProt IDs (Torrente et al., PLoS One 6:e24747, 2011; Kuleg et al., Mol. Cell. Prot. 16:S92-S107, 2017). Because published datasets differ in their analytical depths from our iDAPT-MS datasets, we converted gene identifiers to Human Protein Atlas subcellular enrichment proportions for better comparison. Specifically, the proportion for each subcellular localization term and for each dataset was calculated as the (number of proteins overlapping between the subcellular term and the dataset)/(number of proteins overlapping between all annotated Human Protein Atlas proteins and the dataset). These proportions were used as features for principal component analysis.

CUT&RUN sample preparation. pAG/MNase (Addgene #123461) was expressed in Rosetta2 cells (EMD Millipore), purified with the Pierce His Protein Interaction Pull-Down kit (Thermo), and stored at either −80° C. for long-term storage or −20° C. for working stocks (Meers et al., Elife 8, 2019). CUT&RUN was performed similarly as previously reported (Skene et al., Elife 6, 2017). 500,000 K562 cells per assay were washed three times (room temperature, 3 minutes, 600×g) in wash buffer (20 mM HEPES pH 7.5, 150 mM NaCl, 0.5 mM spermidine, and 1× cOmplete EDTA-free protease inhibitor cocktail [Roche]). Concavalin A beads were activated by washing beads in binding buffer (20 μM HEPES pH 7.5, 10 mM KCl, 1 mM CaCl₂), 1 mM MnCl₂). 10 μL activated Concavalin A beads were added to 100 μL cell suspension and incubated with rotation for 10 minutes at room temperature. Supernatant was removed, and 100 μL wash buffer containing 0.01% digitonin (dig-wash buffer) was added. Antibodies were added at 1:50 concentration, and tubes were incubated with rotation overnight at 4° C. Beads were washed with dig-wash buffer, pAG/MNase was added at a final concentration of 2 μg/mL, and suspensions were incubated for 1 hour at 4° C. Beads were further washed with wash buffer, resuspended in 100 μL wash buffer, and chilled to 0° C. in an ice-water bath. 2 μL 0.1 M CaCl₂) was added to each tube, and tubes were incubated for 1 hour at 0° C. 100 μL stop buffer (340 mM NaCl, 20 mM EDTA, 4 mM EGTA, 0.05% digitonin, 100 μg/mL RNase A, 50 μg/mL GlycoBlue) was added, and tubes were incubated for 15 minutes 37° C. to release DNA fragments. Supernatant was collected, SDS (0.1% final) and proteinase K (250 μg/mL final) were added to each 200 μL sample, and tubes were incubated for 1 hour at 50° C. DNA was isolated by phenol/chloroform extraction, and libraries were constructed using the NEBNext Ultra kit (NEB) as previously described (Liu et al., Cell 173:430-442.e17, 2018). Libraries were then subject to TapeStation 2200 High Sensitivity D1000 fragment size analysis (Agilent) and NextSeq 500 High Output paired-end sequencing (2×42 bp, Illumina). Primary antibodies used for CUT&RUN were: ERH (Bethyl, A305-402A; 1:50), WBP11 (Bethyl, A304-855A; 1:50), and normal rabbit IgG (EMD Millipore, #12-370; 1:50).

Antibodies used for CUT&RUN were validated by immunoprecipitation followed by Western blotting analysis. K562 cells were lysed in RIPA, and 1.5 μL antibody was added to 500 μg protein lysate and incubated overnight at 4° C. The next day, lysates were incubated with 20 μL Pierce protein A magnetic beads (Thermo) for 2 hours at 4° C., beads were washed in RIPA buffer, and bound protein was boiled in 2×LDS sample buffer for 10 minutes. Resulting protein lysates were subjected to Western blotting analysis as described above. Primary antibodies used for Western blotting were: ERH (Atlas Antibodies, HPA002567; 1:1,000) and WBP11 (Bethyl, A304-857A; 1:1,000).

CUT&RUN analysis. Paired-end sequencing reads were trimmed with TrimGalore v0.4.5 to remove adaptor sequence GTGACTGGAGTTCAGACGTGTGCTCTTCCGATCT (SEQ ID NO: 40) with additional removal of fragments smaller than 25 bp. Reads were aligned to the hg38 reference genome using bowtie2 v2.2.9 with options “--no-unal--no-discordant--no-mixed--dovetail-I 25-X 700.” Reads mapping to the mitochondrial genome were subsequently removed, and duplicate reads were removed with Picard v2.8.0. Reads smaller than 120 bp were retained for subsequent analysis. Visualization was performed by mapping insertions to a genome-wide sliding 150 bp window with 20 bp offsets with bedops v2.4.30, followed by conversion to bigwig format with wigToBigWig from UCSC tools v363. Genome tracks were visualized with Integrative Genomics Viewer v2.5.0. Open chromatin regions were defined as 1% FDR-thresholded MACS2 peaks obtained from K562 iDAPT-seq relative to genomic DNA input as described above. CUT&RUN signal was determined relative to these peak regions and normalized by the signal intensity between +1950 and +2000 bp distal to the peak summit, representing background enrichment. CUT&RUN peaks were called by MACS2 v2.1.1 using options “callpeak-q 0.01--keep-dup all.” CUT&RUN and ChIP-seq peak overlap analyses were performed with bedtools v2.26.0 using the intersect function.

ATAC-seq/iDAPT-seq transcription factor analysis. Motif enrichment analysis was performed with ChromVAR as previously described using the human_pwms_v2 set of curated CisBP transcription factor motifs (Weirauch et al., Cell 158:1431-1443, 2014; Schep et al., Nat. Methods 14:975-978, 2017). ChromVAR motif deviations from the computeDeviations function were used for principal component analysis, and FDR-adjusted p-values were obtained with the differentialDeviations function with default settings.

Bivariate footprinting analysis was performed similarly as previously described with slight modifications (Baek et al., Cell Rep. 19:1710-1722, 2017; Corces et al., Science 362 (6413), 2018). CisBP motifs curated from the ChromVAR human_pwms_v2 dataset (Weirauch et al., Cell 158:1431-1443, 2014; Schep et al., Nat. Methods 14:975-978, 2017) or motifs for ZEB2 (Heinz et al., Mol. Cell 38:576-589, 2010) and EBF3 (Fornes et al., Nuc. Acids Res., doi:10:1093/nar/gkz1001, 2019) were matched within peaks using matchMotifs from motifmatchr in R. Motif alignments were extended by 250 bp on each side, and adjusted transposon insertions were mapped to the corresponding regions. Motif flank height was determined by the average insertion rate between positions +1 to +50 bp, immediately flanking the motif. Background insertions were determined by the average insertion rate between positions +200 to +250 bp, distal to the positioned motif. Footprint height was determined by the 10% trimmed mean of the insertion rate within the 10-11 bp positioned around the center of the motif. Footprint depth (FPD) was determined as the log 2 count ratio of footprint height over flank height; flanking accessibility (FA) was determined as the log 2 count ratio of flank height over background. The norm of the orthogonal projection of FA and FPD scores onto the −45° line was used as a raw footprinting score. A linear regression model was implemented (footprinting score˜transcription factor+transcription factor:treatment), from which the t-statistic of the interaction term per transcription factor motif (transcription factor:treatment) was used as the composite footprinting score, and the corresponding p-value, adjusted to false discovery rate with the Benjamini-Hochberg method, was used to assess significance.

For analysis of transcription factor activity at steady-state, composite footprinting scores were modeled by a two-state Gaussian mixture model with mixtools in R, and class A footprinted motifs (strong footprinting) were determined to be those with greater than 50% probability of being in the Gaussian distribution further away from the origin. Class C footprinted motifs (no/negative footprinting) were determined as those with weak statistical significance (FDR >5%) or negative enrichment (composite footprinting score <0). Positive and significant footprinted motifs not in class A were demarcated as class B footprinted motifs (weak footprinting). Consensus transcription factor classifications were determined by concordance between K562 and NB4 steady-state footprinting analyses, limited to those transcription factors exhibiting positive significant enrichment from both corresponding iDAPT-MS datasets.

For classification of transcription factors upon ATRA treatment, FDR <5% thresholds of iDAPT-MS abundance and iDAPT-seq footprinting profiles were used to discriminate between classes.

ChIP-seq analysis. ENCODE ChIP-seq transcription factor datasets were downloaded from the ENCODE data portal (Encode Consortium, Nature 489:57-74, 2012) (www.encodeproject.org). In brief, ChIP-seq bed files aligned to hg38 and annotated as “optimal IDR peaks” were downloaded, and iDAPT-seq peaks overlapping with ChIP-seq peaks were collated. ChIP-seq enrichment within open chromatin was determined by gene set enrichment analysis using iDAPT-seq differential peaks ranked by log 2 fold change using the fgsea package in R.

Colocalization of ChIP-seq epitopes on open chromatin was determined using the Jaccard similarity coefficient, with colocalization determined if ChIP-seq peaks from different epitopes overlap a given iDAPT-seq peak.

Granulocytic differentiation analysis. NB4 cells treated either with DMSO or 1 μM ATRA were washed with 2% fetal bovine serum prior to staining. Anti-human CD11b-PE-Cy7 antibody conjugate (Clone: ICRF44, Biolegend Catalog #301321; 1:100) and anti-human CD11c-APC antibody conjugate (Clone: B-1y6, BD Pharmingen #559877; 1:100) were incubated with samples for 20 minutes and then washed to remove excess antibody. Stained samples were analyzed on a Beckman Coulter CytoFLEX LX flow cytometer with the CytoExpert v2.3.1.22 software. Data were analyzed with FlowJo v10.0.7.

Cell proliferation assay. NB4 cells were seeded at a density of 5e5 cells/mL subjected to either DMSO or 1 μM ATRA. After 48 hours, 50 μL cell suspension was added to 50 μL CellTiter-Glo reagent, incubated for 10 minutes at room temperature, and assayed for luminescence with a SpectraMax iD3 plate reader.

Genetic dependency analysis. Genetic dependency map (DepMap) scores generated from CRISPR/Cas9 pooled screening (Avana) were downloaded (19Q3, https://depmap.org/portal/). DepMap scores from hematopoietic cancer cell lines were collated, and the distribution of dependency scores was modeled as a two-state Gaussian mixture model with mixtools in R. Gene dependency was determined as the threshold corresponding to 50% probability of being in either distribution. Essential genes across hematopoietic cell lines were those genes representing dependencies across at least 50% of profiled hematopoietic cell lines.

RNA-seq analysis. Raw sequencing reads (GSM1288651, GSM1288652, GSM1288653, GSM1288654, GSM1288659, GSM1288660, GSM1288661, GSM1288662, GSM2464389, GSM2464392) were aligned to a reference transcriptome generated from the Ensembl v94 database with salmon v0.14.1 using options “--seqBias--useVBOpt--gcBias--posBias--numBootstraps 30-validateMappings.” Length-scaled transcripts per million were acquired using the tximport function, and log 2 fold changes and false discovery rates were determined by DESeq2 in R, with batch as a covariate. Principal component analysis was performed with counts transformed by the varianceStabilizingTransformation function from DESeq2, and shrunken log 2 fold changes were determined with DESeq2, which were used to rank genes for gene set enrichment analysis. For comparison of RNA-seq and mass spectrometry datasets, gene symbols and Ensembl gene IDs were matched to UniProt IDs via biomaRt.

Statistical analysis. No statistical methods were used to predetermine sample size. The experiments were not randomized. The investigators were not blinded to allocation during experiments and outcome assessment. All statistical analyses were performed in R (R Core Team. R: A language for statistical computing, 2014). Two-tailed statistical tests were used unless stated otherwise. Multiple comparison adjustments were performed as noted.

### Sequence Information

Tn5 Transposase
ATGATTACCAGTGCACTGCATCGTGCGGCGGATTGGGCGAAAAGCGTGTTTTCTAGTGCTGCGCTG
GGTGATCCGCGTCGTACCGCGCGTCTGGTGAATGTTGCGGCGCAACTGGCCAAATATAGCGGCAAA
AGCATTACCATTAGCAGCGAAGGCAGCAAAGCCATGCAGGAAGGCGCGTATCGTTTTATTCGTAATC
CGAACGTGAGCGCGGAAGCGATTCGTAAAGCGGGTGCCATGCAGACCGTGAAACTGGCCCAGGAA
TTTCCGGAACTGCTGGCAATTGAAGATACCACCTCTCTGAGCTATCGTCATCAGGTGGCGGAAGAAC
TGGGCAAACTGGGTAGCATTCAGGATAAAAGCCGTGGTTGGTGGGTGCATAGCGTGCTGCTGCTGG
AAGCGACCACCTTTCGTACCGTGGGCCTGCTGCATCAAGAATGGTGGATGCGTCCGGATGATCCGG
CGGATGCGGATGAAAAAGAAAGCGGCAAATGGCTGGCCGCTGCTGCAACTTCGCGTCTGAGAATGG
GCAGCATGATGAGCAACGTGATTGCGGTGTGCGATCGTGAAGCGGATATTCATGCGTATCTGCAAG
ATAAACTGGCCCATTAACGAACGTTTTGTGGTGCGTAGCAAACATCCGCGTAAAGATGTGGAAAGCGG
CCTGTATCTGTATGATCACCTGAAAAACCAGCCGGAACTGGGCGGCTATCAGATTAGCATTCCGCAG
AAAGGCGTGGTGGATAAACGTGGCAAACGTAAAAACCGTCCGGCGCGTAAAGCGAGCCTGAGCCTG
CGTAGCGGCCGTATTACCCTGAAACAGGGCAACATTACCCTGAACGCGGTGCTGGCCGAAGAAATT
AATCCGCCGAAAGGCGAAACCCCGCTGAAATGGCTGCTGCTGACCAGCGAGCCGGTGGAAAGTCT
GGCCCAAGCGCTGCGTGTGATTGATATTTATACCCATCGTTGGCGCATTGAAGAATTTCACAAAGCG
TGGAAAACGGGTGCGGGTGCGGAACGTCAGCGTATGGAAGAACCGGATAACCTGGAACGTATGGT
GAGCATTCTGAGCTTTGTGGCGGTGCGTCTGCTGCAACTGCGTGAATCTTTTACTCCGCCGCAAGCA
CTGCGTGCGCAGGGCCTGCTGAAAGAAGCGGAACACGTTGAAAGCCAGAGCGCGGAAACCGTGCT
GACCCCGGATGAATGCCAACTGCTGGGCTATCTGGATAAAGGCAAACGCAAACGCAAAGAAAAAGC
GGGCAGCCTGCAATGGGCGTATATGGCGATTGCGCGTCTGGGCGGCTTTATGGATAGCAAACGTAC
CGGCATTGCGAGCTGGGGTGCGCTGTGGGAAGGTTGGGAAGCGCTGCAAAGCAAACTGGATGGCT
TTCTGGCCGCGAAAGACCTGATGGCGCAGGGCATTAAAATC (SEQ ID NO: 1) (from Picelli et al.:
genome.cshlp.org/content/24/12/2033.full.html; Addgene: #60240, addgene.org/60240/)
MITSALHRAADWAKSVFSSAALGDPRRTARLVNVAAQLAKYSGKSITISSEGSKAMQEGAYRFIRNPNVS
AEAIRKAGAMQTVKLAQEFPELLAIEDTTSLSYRHQVAEELGKLGSIQDKSRGWWVHSVLLLEATTFRTV
GLLHQEWWMRPDDPADADEKESGKWLAAAATSRLRMGSMMSNVIAVCDREADIHAYLQDKLAHNERF
VRSKHPRKDVESGLYLYDHLKNQPELGGYQISIPQKGVVDKRGKRKNRPARKASLSLRSGRITLKQGNI
TLNAVLAEEINPPKGETPLKWLLLTSEPVESLAQALRVIDIYTHRWRIEEFHKAWKTGAGAERQRMEEPDN
LERMVSILSFVAVRLLQLRESFTPPQALRAQGLLKEAEHVESQSAETVLTPDECQLLGYLDKGKRKRKEK
AGSLQWAYMAIARLGGFMDSKRTGIASWGALWEGWEALQSKLDGFLAAKDLMAQGIKI (SEQ ID NO: 2)
APEX2
GGAAAGTCTTACCCAACTGTGAGTGCTGATTACCAGGACGCCGTTGAGAAGGCGAAGAAGAAGCTC
AGAGGCTTCATCGCTGAGAAGAGATGCGCTCCTCTAATGCTCCGTTTGGCATTCCACTCTGCTGGAA
CCTTTGACAAGGGCACGAAGACCGGTGGACCCTTCGGAACCATCAAGCACCCTGCCGAACTGGCTC
ACAGCGCTAACAACGGTCTTGACATCGCTGTTAGGCTTTTGGAGCCACTCAAGGCGGAGTTCCCTAT
TTTGAGCTACGCCGATTTCTACCAGTTGGCTGGCGTTGTTGCCGTTGAGGTCACGGGTGGACCTAA
GGTTCCATTCCACCCTGGAAGAGAGGACAAGCCTGAGCCACCACCAGAGGGTCGCTTGCCCGATCC
CACTAAGGGTTCTGACCATTTGAGAGATGTGTTTGGCAAAGCTATGGGGCTTACTGACCAAGATATC
GTTGCTCTATCTGGGGGTCACACTATTGGAGCTGCACACAAGGAGCGTTCTGGATTTGAGGGTCCCT
GGACCTCTAATCCTCTTATTTTCGACAACTCATACTTCACGGAGTTGTTGAGTGGTGAGAAGGAAGGT
CTCCTTCAGCTACCTTCTGACAAGGCTCTTTTGTCTGACCCTGTATTCCGCCCTCTCGTTGACAAATA
TGCAGCGGACGAAGATGCCTTCTTTGCTGATTACGCTGAGGCTCACCAAAAGCTTTCCGAGCTTGGG
TTTGCTGATGCC (SEQ ID NO: 30) (from Lam et al.: nature.com/articles/
nmeth.3179; Addgene: #49386, addgene.org/49386/)
GKSYPTVSADYQDAVEKAKKKLRGFIAEKRCAPLMLRLAFHSAGTFDKGTKTGGPFGTIKHPAELAHSAN
NGLDIAVRLLEPLKAEFPILSYADFYQLAGVVAVEVTGGPKVPFHPGREDKPEPPPEGRLPDPTKGSDHL
RDVFGKAMGLTDQDIVALSGGHTIGAAHKERSGFEGPWTSNPLIFDNSYFTELLSGEKEGLLQLPSDKAL
LSDPVFRPLVDKYAADEDAFFADYAEAHQKLSELGFADA (SEQ ID NO: 4)
APEX
GKSYPTVSADYQDAVEKAKKKLRGFIAEKRCAPLMLRLAFHSAGTFDKGTKTGGPFGTIKHPAELAHSAN
NGLDIAVRLLEPLKAEFPILSYADFYQLAGVVAVEVTGGPKVPFHPGREDKPEPPPEGRLPDATKGSDHL
RDVFGKAMGLTDQDIVALSGGHTIGAAHKERSGFEGPWTSNPLIFDNSYFTELLSGEKEGLLQLPSDKAL
LSDPVFRPLVDKYAADEDAFFADYAEAHQKLSELGFADA (SEQ ID NO: 5)
Linkers
CCAGCTCCAGCTCCA (SEQ ID NO: 6)
PAPAP (SEQ ID NO: 7)
GCTGAGGCTGCTGCTAAGGAGGCTGCTGCTAAGGCG (SEQ ID NO: 8)
AEAAAKEAAAKA (SEQ ID NO: 9)
GGCGGAGGTGGTTCTGGCGGTGGAGGTTCAGGCGGTGGTGGAAGTGGCGGAGGTGGTTCA (SEQ
ID NO: 10)
(GGGGS)₄ (SEQ ID NO: 11)
GGATCCGGTGCAGGCGcc (SEQ ID NO: 12)
GSGAGA (SEQ ID NO: 13)
Tags
Flag Tags
GATTACAAGGATGACGACGATAAG (SEQ ID NO: 14)
DYKDDDDK (SEQ ID NO: 15);
DYKDHDGDYKDHDIDYKDDDDK (SEQ ID NO: 16)
HA Tag
YPYDVPDYA (SEQ ID NO: 17)

Other sequences of the invention are provided below, with sequence identification numbers indicated parenthetically.

| | |
|---|---|
| Tn5-F | ATGATTACCAGTGCACTGCATCGTGCGGCGGATTGGGCGAAAAGCGTGTTTTCTAGTGCTGCGCTGGGTGATCC |
| cDNA | GCGTCGTACCGCGCGTCTGGTGAATGTTGCGGCGCAACTGGCCAAATATAGCGGCAAAAGCATTACCATTAGCA |
| (18) | GCGAAGGCAGCAAAGCCATGCAGGAAGGCGCGTATCGTTTTATTCGTAATCCGAACGTGAGCGCGGAAGCGAT |
| | TCGTAAAGCGGGTGCCATGCAGACCGTGAAACTGGCCCAGGAATTTCCGGAACTGCTGGCAATTGAAGATACCA |
| | CCTCTCTGAGCTATCGTCATCAGGTGGCGGAAGAACTGGGCAAACTGGGTAGCATTCAGGATAAAAGCCGTGGT |
| | TGGTGGGTGCATAGCGTGCTGCTGCTGGAAGCGACCACCTTTCGTACCGTGGGCCTGCTGCATCAAGAATGGT |
| | GGATGCGTCCGGATGATCCGGCGGATGCGGATGAAAAAGAAAGCGGCAAATGGCTGGCCGCTGCTGCAACTTC |
| | GCGTCTGAGAATGGGCAGCATGATGAGCAACGTGATTGCGGTGTGCGATCGTGAAGCGGATATTCATGCGTATC |
| | TGCAAGATAAACTGGCCCATAACGAACGTTTTGTGGTGCGTAGCAAACATCCGCGTAAAGATGTGGAAAGCGGC |
| | CTGTATCTGTATGATCACCTGAAAAACCAGCCGGAACTGGGCGGCTATCAGATTAGCATTCCGCAGAAAGGCGT |
| | GGTGGATAAACGTGGCAAACGTAAAAACCGTCCGGCGCGTAAAGCGAGCCTGAGCCTGCGTAGCGGCCGTATT |
| | ACCCTGAAACAGGGCAACATTACCCTGAACGCGGTGCTGGCCGAAGAAATTAATCCGCCGAAAGGCGAAACCC |
| | CGCTGAAATGGCTGCTGCTGACCAGCGAGCCGGTGGAAAGTCTGGCCCAAGCGCTGCGTGTGATTGATATTTAT |
| | ACCCATCGTTGGCGCATTGAAGAATTTCACAAAGCGTGGAAAACGGGTGCGGGTGCGGAACGTCAGCGTATGG |
| | AAGAACCGGATAACCTGGAACGTATGGTGAGCATTCTGAGCTTTGTGGCGGTGCGTCTGCTGCAACTGCGTGAA |
| | TCTTTTACTCCGCCGCAAGCACTGCGTGCGCAGGGCCTGCTGAAAGAAGCGGAACACGTTGAAAGCCAGAGCG |
| | CGGAAACCGTGCTGACCCCGGATGAATGCCAACTGCTGGGCTATCTGGATAAAGGCAAACGCAAACGCAAAGA |
| | AAAAGCGGGCAGCCTGCAATGGGCGTATATGGCGATTGCGCGTCTGGGCGGCTTTATGGATAGCAAACGTACC |
| | GGCATTGCGAGCTGGGGTGCGCTGTGGGAAGGTTGGGAAGCGCTGCAAAGCAAACTGGATGGCTTTCTGGCC |
| | GCGAAAGACCTGATGGCGCAGGGCATTAAAATCgattacaaggatgacgacgataag |
| Tn5-F | MITSALHRAADWAKSVFSSAALGDPRRTARLVNVAAQLAKYSGKSITISSEGSKAMQEGAYRFIRNPNVSAEAIRKAG |
| amino | AMQTVKLAQEFPELLAIEDTTSLSYRHQVAEELGKLGSIQDKSRGWWVHSVLLLEATTFRTVGLLHQEVWVMRPDDPA |
| acid | DADEKESGKWLAAAATSRLRMGSMMSNVIAVCDREADIHAYLQDKLAHNERFWRSKHPRKDVESGLYLYDHLKNQ |
| (19) | PELGGYQISIPQKGVVDKRGKRKNRPARKASLSLRSGRITLKQGNITLNAVLAEEINPPKGETPLKWLLLTSEPVESLA |
| | QALRVIDIYTHRWRIEEFHKAWKTGAGAERQRMEEPDNLERMVSILSFVAVRLLQLRESFTPPQALRAQGLLKEAEHV |
| | ESQSAETVLTPDECQLLGYLDKGKRKRKEKAGSLQWAYMAIARLGGFMDSKRTGIASWGALWEGWEALQSKLDGFL |
| | AAKDLMAQGIKIDYKDDDDK |
| APEX2- | ATGggaaagtcttacccaactgtgagtgctgattaccaggacgccgttgagaaggcgaagaagaagctcagaggcttcatcgct |
| F | gagaagagatgcgctcctctaatgctccgtttggcattccactctgctggaacctttgacaagggcacgaagaccggtggaccc |
| cDNA | ttcggaaccatcaagcaccctgccgaactggctcacagcgctaacaacggtcttgacatcgctgttaggcttttggagccact |
| (20) | caaggcggagttccctattttgagctacgccgatttctaccagttggctggcgttgttgccgttgaggtcacgggtggacctaa |
| | ggttccattccaccctggaagagaggacaagcctgagccaccaccagagggtcgcttgcccgatcccactaagggttctgacca |
| | tttgagagatgtgtttggcaaagctatggggcttactgaccaagatatcgttgctctatctgggggtcacactattggagctg |
| | cacacaaggagcgttctggatttgagggtccctggacctctaatcctcttattttcgacaactcatacttcacggagttgttg |
| | agtggtgagaaggaaggtctccttcagctaccttctgacaaggctcttttgtctgaccctgtattccgccctctcgttgacaa |
| | atatgcagcggacgaagatgccttctttgctgattacgctgaggctcaccaaaagctttccgagcttgggtttgctgatgccg |
| | attacaaggatgacgacgataag |
| APEX2- | MGKSYPTVSADYQDAVEKAKKKLRGFIAEKRCAPLMLRLAFHSAGTFDKGTKTGGPFGTIKHPAELAHSANNGLDIAV |
| F | RLLEPLKAEFPILSYADFYQLAGVVAVEVTGGPKVPFHPGREDKPEPPPEGRLPDPTKGSDHLRDVFGKAMGLTDQDI |
| amino | VALSGGHTIGAAHKERSGFEGPWTSNPLIFDNSYFTELLSGEKEGLLQLPSDKALLSDPVFRPLVDKYAADEDAFFAD |
| acid | YAEAHQKLSELGFADADYKDDDDK |
| (21) | |
| TP1 | ATGATTACCAGTGCACTGCATCGTGCGGCGGATTGGGCGAAAAGCGTGTTTTCTAGTGCTGCGCTGGGTGATCC |
| cDNA | GCGTCGTACCGCGCGTCTGGTGAATGTTGCGGCGCAACTGGCCAAATATAGCGGCAAAAGCATTACCATTAGCA |
| (22) | GCGAAGGCAGCAAAGCCATGCAGGAAGGCGCGTATCGTTTTATTCGTAATCCGAACGTGAGCGCGGAAGCGAT |
| | TCGTAAAGCGGGTGCCATGCAGACCGTGAAACTGGCCCAGGAATTTCCGGAACTGCTGGCAATTGAAGATACCA |
| | CCTCTCTGAGCTATCGTCATCAGGTGGCGGAAGAACTGGGCAAACTGGGTAGCATTCAGGATAAAAGCCGTGGT |
| | TGGTGGGTGCATAGCGTGCTGCTGCTGGAAGCGACCACCTTTCGTACCGTGGGCCTGCTGCATCAAGAATGGT |
| | GGATGCGTCCGGATGATCCGGCGGATGCGGATGAAAAAGAAAGCGGCAAATGGCTGGCCGCTGCTGCAACTTC |
| | GCGTCTGAGAATGGGCAGCATGATGAGCAACGTGATTGCGGTGTGCGATCGTGAAGCGGATATTCATGCGTATC |
| | TGCAAGATAAACTGGCCCATAACGAACGTTTTGTGGTGCGTAGCAAACATCCGCGTAAAGATGTGGAAAGCGGC |
| | CTGTATCTGTATGATCACCTGAAAAACCAGCCGGAACTGGGCGGCTATCAGATTAGCATTCCGCAGAAAGGCGT |
| | GGTGGATAAACGTGGCAAACGTAAAAACCGTCCGGCGCGTAAAGCGAGCCTGAGCCTGCGTAGCGGCCGTATT |
| | ACCCTGAAACAGGGCAACATTACCCTGAACGCGGTGCTGGCCGAAGAAATTAATCCGCCGAAAGGCGAAACCC |
| | CGCTGAAATGGCTGCTGCTGACCAGCGAGCCGGTGGAAAGTCTGGCCCAAGCGCTGCGTGTGATTGATATTTAT |
| | ACCCATCGTTGGCGCATTGAAGAATTTCACAAAGCGTGGAAAACGGGTGCGGGTGCGGAACGTCAGCGTATGG |
| | AAGAACCGGATAACCTGGAACGTATGGTGAGCATTCTGAGCTTTGTGGCGGTGCGTCTGCTGCAACTGCGTGAA |
| | TCTTTTACTCCGCCGCAAGCACTGCGTGCGCAGGGCCTGCTGAAAGAAGCGGAACACGTTGAAAGCCAGAGCG |
| | CGGAAACCGTGCTGACCCCGGATGAATGCCAACTGCTGGGCTATCTGGATAAAGGCAAACGCAAACGCAAAGA |
| | AAAAGCGGGCAGCCTGCAATGGGCGTATATGGCGATTGCGCGTCTGGGCGGCTTTATGGATAGCAAACGTACC |
| | GGCATTGCGAGCTGGGGTGCGCTGTGGGAAGGTTGGGAAGCGCTGCAAAGCAAACTGGATGGCTTTCTGGCC |
| | GCGAAAGACCTGATGGCGCAGGGCATTAAAATCggaaagtcttacccaactgtgagtgctgattaccaggacgccgttgagaag |
| | gcgaagaagaagctcagaggcttcatcgctgagaagagatgcgctcctctaatgctccgtttggcattccactctgctggaac |
| | ctttgacaagggcacgaagaccggtggacccttcggaaccatcaagcaccctgccgaactggctcacagcgctaacaacggtc |
| | ttgacatcgctgttaggcttttggagccactcaaggcggagttccctattttgagctacgccgatttctaccagttggctgg |
| | cgttgttgccgttgaggtcacgggtggacctaaggttccattccaccctggaagagaggacaagcctgagccaccaccagag |
| | ggtcgcttgcccgatcccactaagggttctgaccatttgagagatgtgtttggcaaagctatggggcttactgaccaagatat |
| | cgttgctctatctgggggtcacactattggagctgcacacaaggagcgttctggatttgagggtccctggacctctaatcct |
| | cttattttcgacaactcatacttcacggagttgttgagtggtgagaaggaaggtctccttcagctaccttctgacaaggctct |
| | tttgtctgaccctgtattccgccctctcgttgacaaatatgcagcggacgaagatgccttctttgctgattacgctgaggctc |
| | accaaaagctttccgagcttgggtttgctgatgccgattacaaggatgacgacg ataag |
| TP1 | MITSALHRAADWAKSVFSSAALGDPRRTARLVNVAAQLAKYSGKSITISSEGSKAMQEGAYRFIRNPNVSAEAIRKAG |
| amino | AMQTVKLAQEFPELLAIEDTTSLSYRHQVAEELGKLGSIQDKSRGWWVHSVLLLEATTFRTVGLLHQEWWMRPDDPA |
| acid | DADEKESGKWLAAAATSRLRMGSMMSNVIAVCDREADIHAYLQDKLAHNERFWRSKHPRKDVESGLYLYDHLKNQ |
| (23) | PELGGYQISIPQKGVVDKRGKRKNRPARKASLSLRSGRITLKQGNITLNAVLAEEINPPKGETPLKWLLLTSEPVESLA |
| | QALRVIDIYTHRWRIEEFHKAWKTGAGAERQRMEEPDNLERMVSILSFVAVRLLQLRESFTPPQALRAQGLLKEAEHV |
| | ESQSAETVLTPDECQLLGYLDKGKRKRKEKAGSLQWAYMAIARLGGFMDSKRTGIASWGALWEGWEALQSKLDGFL |
| | AAKDLMAQGIKIGKSYPTVSADYQDAVEKAKKKLRGFIAEKRCAPLMLRLAFHSAGTFDKGTKTGGPFGTIKHPAELAH |
| | SANNGLDIAVRLLEPLKAEFPILSYADFYQLAGVVAVEVTGGPKVPFHPGREDKPEPPPEGRLPDPTKGSDHLRDVFG |
| | KAMGLTDQDIVALSGGHTIGAAHKERSGFEGPWTSNPLIFDNSYFTELLSGEKEGLLQLPSDKALLSDPVFRPLVDKY |
| | AADEDAFFADYAEAHQKLSELGFADADYKDDDDK |
| TP2 | ATGATTACCAGTGCACTGCATCGTGCGGCGGATTGGGCGAAAAGCGTGTTTTCTAGTGCTGCGCTGGGTGATCC |
| cDNA | GCGTCGTACCGCGCGTCTGGTGAATGTTGCGGCGCAACTGGCCAAATATAGCGGCAAAAGCATTACCATTAGCA |
| (24) | GCGAAGGCAGCAAAGCCATGCAGGAAGGCGCGTATCGTTTTATTCGTAATCCGAACGTGAGCGCGGAAGCGAT |
| | TCGTAAAGCGGGTGCCATGCAGACCGTGAAACTGGCCCAGGAATTTCCGGAACTGCTGGCAATTGAAGATACCA |
| | CCTCTCTGAGCTATCGTCATCAGGTGGCGGAAGAACTGGGCAAACTGGGTAGCATTCAGGATAAAAGCCGTGGT |
| | TGGTGGGTGCATAGCGTGCTGCTGCTGGAAGCGACCACCTTTCGTACCGTGGGCCTGCTGCATCAAGAATGGT |
| | GGATGCGTCCGGATGATCCGGCGGATGCGGATGAAAAAGAAAGCGGCAAATGGCTGGCCGCTGCTGCAACTTC |
| | GCGTCTGAGAATGGGCAGCATGATGAGCAACGTGATTGCGGTGTGCGATCGTGAAGCGGATATTCATGCGTATC |
| | TGCAAGATAAACTGGCCCATAACGAACGTTTTGTGGTGCGTAGCAAACATCCGCGTAAAGATGTGGAAAGCGGC |
| | CTGTATCTGTATGATCACCTGAAAAACCAGCCGGAACTGGGCGGCTATCAGATTAGCATTCCGCAGAAAGGCGT |
| | GGTGGATAAACGTGGCAAACGTAAAAACCGTCCGGCGCGTAAAGCGAGCCTGAGCCTGCGTAGCGGCCGTATT |
| | ACCCTGAAACAGGGCAACATTACCCTGAACGCGGTGCTGGCCGAAGAAATTAATCCGCCGAAAGGCGAAACCC |
| | CGCTGAAATGGCTGCTGCTGACCAGCGAGCCGGTGGAAAGTCTGGCCCAAGCGCTGCGTGTGATTGATATTTAT |
| | ACCCATCGTTGGCGCATTGAAGAATTTCACAAAGCGTGGAAAACGGGTGCGGGTGCGGAACGTCAGCGTATGG |
| | AAGAACCGGATAACCTGGAACGTATGGTGAGCATTCTGAGCTTTGTGGCGGTGCGTCTGCTGCAACTGCGTGAA |
| | TCTTTTACTCCGCCGCAAGCACTGCGTGCGCAGGGCCTGCTGAAAGAAGCGGAACACGTTGAAAGCCAGAGCG |
| | CGGAAACCGTGCTGACCCCGGATGAATGCCAACTGCTGGGCTATCTGGATAAAGGCAAACGCAAACGCAAAGA |
| | AAAAGCGGGCAGCCTGCAATGGGCGTATATGGCGATTGCGCGTCTGGGCGGCTTTATGGATAGCAAACGTACC |
| | GGCATTGCGAGCTGGGGTGCGCTGTGGGAAGGTTGGGAAGCGCTGCAAAGCAAACTGGATGGCTTTCTGGCC |
| | GCGAAAGACCTGATGGCGCAGGGCATTAAAATCCCAGCTCCAGCTCCAggaaagtcttacccaactgtgagtgctgattaccag |
| | gacgccgttgagaaggcgaagaagaagctcagaggcttcatcgctgagaagagatgcgctcctctaatgctccgtttggcat |
| | tccactctgctggaacctttgacaagggcacgaagaccggtggacccttcggaaccatcaagcaccctgccgaactggctca |
| | cagcgctaacaacggtcttgacatcgctgttaggcttttggagccactcaaggcggagttccctattttgagctacgccgatt |
| | tctaccagttggctggcgttgttgccgttgaggtcacgggtggacctaaggttccattccaccctggaagagaggacaagcct |
| | gagccaccaccagagggtcgcttgcccgatcccactaagggttctgaccatttgagagatgtgtttggcaaagctatggggc |
| | ttactgaccaagatatcgttgctctatctgggggtcacactattggagctgcacacaaggagcgttctggatttgagggtccc |
| | tggacctctaatcctcttattttcgacaactcatacttcacggagttgttgagtggtgagaaggaaggtctccttcagctacc |
| | ttctgacaaggctcttttgtctgaccctgtattccgccctctcgttgacaaatatgcagcggacgaagatgccttctttgctg |
| | attacgctgaggctcaccaaaagctttccgagcttgggtttgctgatgccgattacaaggatgacgacgataag |
| TP2 | MITSALHRAADWAKSVFSSAALGDPRRTARLVNVAAQLAKYSGKSITISSEGSKAMQEGAYRFIRNPNVSAEAIRKAG |
| amino | AMQTVKLAQEFPELLAIEDTTSLSYRHQVAEELGKLGSIQDKSRGWWVHSVLLLEATTFRTVGLLHQEVWVMRPDDPA |
| acid | DADEKESGKWLAAAATSRLRMGSMMSNVIAVCDREADIHAYLQDKLAHNERFVVRSKHPRKDVESGLYLYDHLKNQ |
| (25) | PELGGYQISIPQKGVVDKRGKRKNRPARKASLSLRSGRITLKQGNITLNAVLAEEINPPKGETPLKWLLLTSEPVESLA |
| | QALRVIDIYTHRWRIEEFHKAWKTGAGAERQRMEEPDNLERMVSILSFVAVRLLQLRESFTPPQALRAQGLLKEAEHV |
| | ESQSAETVLTPDECQLLGYLDKGKRKRKEKAGSLQWAYMAIARLGGFMDSKRTGIASWGALWEGWEALQSKLDGFL |
| | AAKDLMAQGIKIPAPAPGKSYPTVSADYQDAVEKAKKKLRGFIAEKRCAPLMLRLAFHSAGTFDKGTKTGGPFGTIKH |
| | PAELAHSANNGLDIAVRLLEPLKAEFPILSYADFYQLAGWAVEVTGGPKVPFHPGREDKPEPPPEGRLPDPTKGSDH |
| | LRDVFGKAMGLTDQDIVALSGGHTIGAAHKERSGFEGPWTSNPLIFDNSYFTELLSGEKEGLLQLPSDKALLSDPVFR |
| | PLVDKYAADEDAFFADYAEAHQKLSELGFADADYKDDDDK |
| TP3 | ATGATTACCAGTGCACTGCATCGTGCGGCGGATTGGGCGAAAAGCGTGTTTTCTAGTGCTGCGCTGGGTGATCC |
| cDNA | GCGTCGTACCGCGCGTCTGGTGAATGTTGCGGCGCAACTGGCCAAATATAGCGGCAAAAGCATTACCATTAGCA |
| (26) | GCGAAGGCAGCAAAGCCATGCAGGAAGGCGCGTATCGTTTTATTCGTAATCCGAACGTGAGCGCGGAAGCGAT |
| | TCGTAAAGCGGGTGCCATGCAGACCGTGAAACTGGCCCAGGAATTTCCGGAACTGCTGGCAATTGAAGATACCA |
| | CCTCTCTGAGCTATCGTCATCAGGTGGCGGAAGAACTGGGCAAACTGGGTAGCATTCAGGATAAAAGCCGTGGT |
| | TGGTGGGTGCATAGCGTGCTGCTGCTGGAAGCGACCACCTTTCGTACCGTGGGCCTGCTGCATCAAGAATGGT |
| | GGATGCGTCCGGATGATCCGGCGGATGCGGATGAAAAAGAAAGCGGCAAATGGCTGGCCGCTGCTGCAACTTC |
| | GCGTCTGAGAATGGGCAGCATGATGAGCAACGTGATTGCGGTGTGCGATCGTGAAGCGGATATTCATGCGTATC |
| | TGCAAGATAAACTGGCCCATAACGAACGTTTTGTGGTGCGTAGCAAACATCCGCGTAAAGATGTGGAAAGCGGC |
| | CTGTATCTGTATGATCACCTGAAAAACCAGCCGGAACTGGGCGGCTATCAGATTAGCATTCCGCAGAAAGGCGT |
| | GGTGGATAAACGTGGCAAACGTAAAAACCGTCCGGCGCGTAAAGCGAGCCTGAGCCTGCGTAGCGGCCGTATT |
| | ACCCTGAAACAGGGCAACATTACCCTGAACGCGGTGCTGGCCGAAGAAATTAATCCGCCGAAAGGCGAAACCC |
| | CGCTGAAATGGCTGCTGCTGACCAGCGAGCCGGTGGAAAGTCTGGCCCAAGCGCTGCGTGTGATTGATATTTAT |
| | ACCCATCGTTGGCGCATTGAAGAATTTCACAAAGCGTGGAAAACGGGTGCGGGTGCGGAACGTCAGCGTATGG |
| | AAGAACCGGATAACCTGGAACGTATGGTGAGCATTCTGAGCTTTGTGGCGGTGCGTCTGCTGCAACTGCGTGAA |
| | TCTTTTACTCCGCCGCAAGCACTGCGTGCGCAGGGCCTGCTGAAAGAAGCGGAACACGTTGAAAGCCAGAGCG |
| | CGGAAACCGTGCTGACCCCGGATGAATGCCAACTGCTGGGCTATCTGGATAAAGGCAAACGCAAACGCAAAGA |
| | AAAAGCGGGCAGCCTGCAATGGGCGTATATGGCGATTGCGCGTCTGGGCGGCTTTATGGATAGCAAACGTACC |
| | GGCATTGCGAGCTGGGGTGCGCTGTGGGAAGGTTGGGAAGCGCTGCAAAGCAAACTGGATGGCTTTCTGGCC |
| | GCGAAAGACCTGATGGCGCAGGGCATTAAAATCGCTGAGGCTGCTGCTAAGGAGGCTGCTGCTAAGGCGggaaa |
| | gtcttacccaactgtgagtgctgattaccaggacgccgttgagaaggcgaagaagaagctcagaggcttcatcgctgagaaga |
| | gatgcgctcctctaatgctccgtttggcattccactctgctggaacctttgacaagggcacgaagaccggtggacccttcgga |
| | accatcaagcaccctgccgaactggctcacagcgctaacaacggtcttgacatcgctgttaggcttttggagccactcaagg |
| | cggagttccctattttgagctacgccgatttctaccagttggctggcgttgttgccgttgaggtcacgggtggacctaaggtt |
| | ccattccaccctggaagagaggacaagcctgagccaccaccagagggtcgcttgcccgatcccactaagggttctgaccattt |
| | gagagatgtgtttggcaaagctatggggcttactgaccaagatatcgttgctctatctgggggtcacactattggagctgc |
| | acacaaggagcgttctggatttgagggtccctggacctctaatcctcttattttcgacaactcatacttcacggagttgttga |
| | gtggtgagaaggaaggtctccttcagctaccttctgacaaggctcttttgtctgaccctgtattccgccctctcgttgacaa |
| | atatgcagcggacgaagatgccttctttgctgattacgctgaggctcaccaaaagctttccgagcttgggtttgctgatgccg |
| | attacaaggatgacgacgataag |
| TP3 | MITSALHRAADWAKSVFSSAALGDPRRTARLVNVAAQLAKYSGKSITISSEGSKAMQEGAYRFiRNPNVSAEAIRKAG |
| amino | AMQTVKLAQEFPELLAIEDTTSLSYRHQVAEELGKLGSIQDKSRGWWVHSVLLLEATTFRTVGLLHQEVWVMRPDDPA |
| acid | DADEKESGKWLAAAATSRLRMGSMMSNVIAVCDREADIHAYLQDKLAHNERFWRSKHPRKDVESGLYLYDHLKNQ |
| (27) | PELGGYQISIPQKGVVDKRGKRKNRPARKASLSLRSGRITLKQGNITLNAVLAEEINPPKGETPLKWLLLTSEPVESLA |
| | QALRVIDIYTHRWRIEEFHKAWKTGAGAERQRMEEPDNLERMVSILSFVAVRLLQLRESFTPPQALRAQGLLKEAEHV |
| | ESQSAETVLTPDECQLLGYLDKGKRKRKEKAGSLQWAYMAIARLGGFMDSKRTGIASWGALWEGWEALQSKLDGFL |
| | AAKDLMAQGIKIAEAAAKEAAAKAGKSYPTVSADYQDAVEKAKKKLRGFIAEKRCAPLMLRLAFHSAGTFDKGTKTGG |
| | PFGTIKHPAELAHSANNGLDIAVRLLEPLKAEFPILSYADFYQLAGVVAVEVTGGPKVPFHPGREDKPEPPPEGRLPDP |
| | TKGSDHLRDVFGKAMGLTDQDIVALSGGHTIGAAHKERSGFEGPWTSNPLIFDNSYFTELLSGEKEGLLQLPSDKALL |
| | SDPVFRPLVDKYAADEDAFFADYAEAHQKLSELGFADADYKDDDDK |
| TP4 | ATGATTACCAGTGCACTGCATCGTGCGGCGGATTGGGCGAAAAGCGTGTTTTCTAGTGCTGCGCTGGGTGATCC |
| CD NA | GCGTCGTACCGCGCGTCTGGTGAATGTTGCGGCGCAACTGGCCAAATATAGCGGCAAAAGCATTACCATTAGCA |
| (28) | GCGAAGGCAGCAAAGCCATGCAGGAAGGCGCGTATCGTTTTATTCGTAATCCGAACGTGAGCGCGGAAGCGAT |
| | TCGTAAAGCGGGTGCCATGCAGACCGTGAAACTGGCCCAGGAATTTCCGGAACTGCTGGCAATTGAAGATACCA |
| | CCTCTCTGAGCTATCGTCATCAGGTGGCGGAAGAACTGGGCAAACTGGGTAGCATTCAGGATAAAAGCCGTGGT |
| | TGGTGGGTGCATAGCGTGCTGCTGCTGGAAGCGACCACCTTTCGTACCGTGGGCCTGCTGCATCAAGAATGGT |
| | GGATGCGTCCGGATGATCCGGCGGATGCGGATGAAAAAGAAAGCGGCAAATGGCTGGCCGCTGCTGCAACTTC |
| | GCGTCTGAGAATGGGCAGCATGATGAGCAACGTGATTGCGGTGTGCGATCGTGAAGCGGATATTCATGCGTATC |
| | TGCAAGATAAACTGGCCCATAACGAACGTTTTGTGGTGCGTAGCAAACATCCGCGTAAAGATGTGGAAAGCGGC |
| | CTGTATCTGTATGATCACCTGAAAAACCAGCCGGAACTGGGCGGCTATCAGATTAGCATTCCGCAGAAAGGCGT |
| | GGTGGATAAACGTGGCAAACGTAAAAACCGTCCGGCGCGTAAAGCGAGCCTGAGCCTGCGTAGCGGCCGTATT |
| | ACCCTGAAACAGGGCAACATTACCCTGAACGCGGTGCTGGCCGAAGAAATTAATCCGCCGAAAGGCGAAACCC |
| | CGCTGAAATGGCTGCTGCTGACCAGCGAGCCGGTGGAAAGTCTGGCCCAAGCGCTGCGTGTGATTGATATTTAT |
| | ACCCATCGTTGGCGCATTGAAGAATTTCACAAAGCGTGGAAAACGGGTGCGGGTGCGGAACGTCAGCGTATGG |
| | AAGAACCGGATAACCTGGAACGTATGGTGAGCATTCTGAGCTTTGTGGCGGTGCGTCTGCTGCAACTGCGTGAA |
| | TCTTTTACTCCGCCGCAAGCACTGCGTGCGCAGGGCCTGCTGAAAGAAGCGGAACACGTTGAAAGCCAGAGCG |
| | CGGAAACCGTGCTGACCCCGGATGAATGCCAACTGCTGGGCTATCTGGATAAAGGCAAACGCAAACGCAAAGA |
| | AAAAGCGGGCAGCCTGCAATGGGCGTATATGGCGATTGCGCGTCTGGGCGGCTTTATGGATAGCAAACGTACC |
| | GGCATTGCGAGCTGGGGTGCGCTGTGGGAAGGTTGGGAAGCGCTGCAAAGCAAACTGGATGGCTTTCTGGCC |
| | GCGAAAGACCTGATGGCGCAGGGCATTAAAATCggcggaggtggttctggcggtggaggttcaggcggtggtggaagtggcg |
| | gaggtggttcaggaaagtcttacccaactgtgagtgctgattaccaggacgccgttgagaaggcgaagaagaagctcagagg |
| | cttcatcgctgagaagagatgcgctcctctaatgctccgtttggcattccactctgctggaacctttgacaagggcacgaag |
| | accggtggacccttcggaaccatcaagcaccctgccgaactggctcacagcgctaacaacggtcttgacatcgctgttaggc |
| | ttttggagccactcaaggcggagttccctattttgagctacgccgatttctaccagttggctggcgttgttgccgttgag |
| | gtcacgggtggacctaaggttccattccaccctggaagagaggacaagcctgagccaccaccagagggtcgcttgcccgat |
| | cccactaagggttctgaccatttgagagatgtgtttggcaaagctatggggcttactgaccaagatatcgttgctctatctg |
| | ggggtcacactattggagctgcacacaaggagcgttctggatttgagggtccctggacctctaatcctcttattttcgacaa |
| | ctcatacttcacggagttgttgagtggtgagaaggaaggtctccttcagctaccttctgacaaggctcttttgtctgaccct |
| | gtattccgccctctcgttgacaaatatgcagcggacgaagatgccttctttgctgattacgctgaggctcaccaaaagctt |
| | tccgagcttgggtttgctgatgccgattacaaggatgacgacgataag |
| TP4 | MITSALHRAADWAKSVFSSAALGDPRRTARLVNVAAQLAKYSGKSITISSEGSKAMQEGAYRFIRNPNVSAEAIRKAG |
| amino | AMQTVKLAQEFPELLAIEDTTSLSYRHQVAEELGKLGSIQDKSRGWWVHSVLLLEATTFRTVGLLHQEVWVMRPDDPA |
| acid | DADEKESGKWLAAAATSRLRMGSMMSNVIAVCDREADIHAYLQDKLAHNERFVVRSKHPRKDVESGLYLYDHLKNQ |
| (29) | PELGGYQISIPQKGVVDKRGKRKNRPARKASLSLRSGRITLKQGNITLNAVLAEEINPPKGETPLKWLLLTSEPVESLA |
| | QALRVIDIYTHRWRIEEFHKAWKTGAGAERQRMEEPDNLERMVSILSFVAVRLLQLRESFTPPQALRAQGLLKEAEHV |
| | ESQSAETVLTPDECQLLGYLDKGKRKRKEKAGSLQWAYMAIARLGGFMDSKRTGIASWGALWEGWEALQSKLDGFL |
| | AAKDLMAQGIKIGGGGSGGGGSGGGGSGGGGSGKSYPTVSADYQDAVEKAKKKLRGFIAEKRCAPLMLRLAFHSAG |
| | TFDKGTKTGGPFGTIKHPAELAHSANNGLDIAVRLLEPLKAEFPILSYADFYQLAGVVAVEVTGGPKVPFHPGREDKPE |
| | PPPEGRLPDPTKGSDHLRDVFGKAMGLTDQDIVALSGGHTIGAAHKERSGFEGPWTSNPLIFDNSYFTELLSGEKEGL |
| | LQLPSDKALLSDPVFRPLVDKYAADEDAFFADYAEAHQKLSELGFADADYKDDDDK |
| TP5 | ATGATTACCAGTGCACTGCATCGTGCGGCGGATTGGGCGAAAAGCGTGTTTTCTAGTGCTGCGCTGGGTGATCC |
| cDNA | GCGTCGTACCGCGCGTCTGGTGAATGTTGCGGCGCAACTGGCCAAATATAGCGGCAAAAGCATTACCATTAGCA |
| (30) | GCGAAGGCAGCAAAGCCATGCAGGAAGGCGCGTATCGTTTTATTCGTAATCCGAACGTGAGCGCGGAAGCGAT |
| | TCGTAAAGCGGGTGCCATGCAGACCGTGAAACTGGCCCAGGAATTTCCGGAACTGCTGGCAATTGAAGATACCA |
| | CCTCTCTGAGCTATCGTCATCAGGTGGCGGAAGAACTGGGCAAACTGGGTAGCATTCAGGATAAAAGCCGTGGT |
| | TGGTGGGTGCATAGCGTGCTGCTGCTGGAAGCGACCACCTTTCGTACCGTGGGCCTGCTGCATCAAGAATGGT |
| | GGATGCGTCCGGATGATCCGGCGGATGCGGATGAAAAAGAAAGCGGCAAATGGCTGGCCGCTGCTGCAACTTC |
| | GCGTCTGAGAATGGGCAGCATGATGAGCAACGTGATTGCGGTGTGCGATCGTGAAGCGGATATTCATGCGTATC |
| | TGCAAGATAAACTGGCCCATAACGAACGTTTTGTGGTGCGTAGCAAACATCCGCGTAAAGATGTGGAAAGCGGC |
| | CTGTATCTGTATGATCACCTGAAAAACCAGCCGGAACTGGGCGGCTATCAGATTAGCATTCCGCAGAAAGGCGT |
| | GGTGGATAAACGTGGCAAACGTAAAAACCGTCCGGCGCGTAAAGCGAGCCTGAGCCTGCGTAGCGGCCGTATT |
| | ACCCTGAAACAGGGCAACATTACCCTGAACGCGGTGCTGGCCGAAGAAATTAATCCGCCGAAAGGCGAAACCC |
| | CGCTGAAATGGCTGCTGCTGACCAGCGAGCCGGTGGAAAGTCTGGCCCAAGCGCTGCGTGTGATTGATATTTAT |
| | ACCCATCGTTGGCGCATTGAAGAATTTCACAAAGCGTGGAAAACGGGTGCGGGTGCGGAACGTCAGCGTATGG |
| | AAGAACCGGATAACCTGGAACGTATGGTGAGCATTCTGAGCTTTGTGGCGGTGCGTCTGCTGCAACTGCGTGAA |
| | TCTTTTACTCCGCCGCAAGCACTGCGTGCGCAGGGCCTGCTGAAAGAAGCGGAACACGTTGAAAGCCAGAGCG |
| | CGGAAACCGTGCTGACCCCGGATGAATGCCAACTGCTGGGCTATCTGGATAAAGGCAAACGCAAACGCAAAGA |
| | AAAAGCGGGCAGCCTGCAATGGGCGTATATGGCGATTGCGCGTCTGGGCGGCTTTATGGATAGCAAACGTACC |
| | GGCATTGCGAGCTGGGGTGCGCTGTGGGAAGGTTGGGAAGCGCTGCAAAGCAAACTGGATGGCTTTCTGGCC |
| | GCGAAAGACCTGATGGCGCAGGGCATTAAAATCGGATCCGGTGCAGGCGccggaaagtcttacccaactgtgagtgctgattacc |
| | aggacgccgttgagaaggcgaagaagaagctcagaggcttcatcgctgagaagagatgcgctcctctaatgctccgtttggcat |
| | tccactctgctggaacctttgacaagggcacgaagaccggtggacccttcggaaccatcaagcaccctgccgaactggctcac |
| | agcgctaacaacggtcttgacatcgctgttaggcttttggagccactcaaggcggagttccctattttgagctacgccgat |
| | ttctaccagttggctggcgttgttgccgttgaggtcacgggtggacctaaggttccattccaccctggaagagaggacaagc |
| | ctgagccaccaccagagggtcgcttgcccgatcccactaagggttctgaccatttgagagatgtgtttggcaaagctatgggg |
| | cttactgaccaagatatcgttgctctatctgggggtcacactattggagctgcacacaaggagcgttctggatttgagggtc |
| | cctggacctctaatcctcttattttcgacaactcatacttcacggagttgttgagtggtgagaaggaaggtctccttcag |
| | ctaccttctgacaaggctcttttgtctgaccctgtattccgccctctcgttgacaaatatgcagcggacgaagatgccttc |
| | tttgctgattacgctgaggctcaccaaaagctttccgagcttgggtttgctgatgccgattacaaggatgacgacgataag |
| TP5 | MITSALHRAADWAKSVFSSAALGDPRRTARLVNVAAQLAKYSGKSITISSEGSKAMQEGAYRFIRNPNVSAEAIRKAG |
| amino | AMQTVKLAQEFPELLAIEDTTSLSYRHQVAEELGKLGSIQDKSRGWWVHSVLLLEATTFRTVGLLHQEVWVMRPDDPA |
| acid | DADEKESGKWLAAAATSRLRMGSMMSNVIAVCDREADIHAYLQDKLAHNERFWRSKHPRKDVESGLYLYDHLKNQ |
| (31) | PELGGYQISIPQKGVVDKRGKRKNRPARKASLSLRSGRITLKQGNITLNAVLAEEINPPKGETPLKWLLLTSEPVESLA |
| | QALRVIDIYTHRWRIEEFHKAWKTGAGAERQRMEEPDNLERMVSILSFVAVRLLQLRESFTPPQALRAQGLLKEAEHV |
| | ESQSAETVLTPDECQLLGYLDKGKRKRKEKAGSLQWAYMAIARLGGFMDSKRTGIASWGALWEGWEALQSKLDGFL |
| | AAKDLMAQGIKIGSGAGAGKSYPTVSADYQDAVEKAKKKLRGFIAEKRCAPLMLRLAFHSAGTFDKGTKTGGPFGTIK |
| | HPAELAHSANNGLDIAVRLLEPLKAEFPILSYADFYQLAGVVAVEVTGGPKVPFHPGREDKPEPPPEGRLPDPTKGSD |
| | HLRDVFGKAMGLTDQDIVALSGGHTIGAAHKERSGFEGPWTSNPLIFDNSYFTELLSGEKEGLLQLPSDKALLSDPVF |
| | RPLVDKYAADEDAFFADYAEAHQKLSELGFADADYKDDDDK |

### Other Embodiments

Various modifications and variations of the described invention will be apparent to those skilled in the art without departing from the scope and spirit thereof. Although the invention has been described in connection with specific embodiments, it is to be understood that the invention as claimed should not be unduly limited to such specific embodiments. Indeed, various modifications of the described modes for carrying out the invention that are obvious to those skilled in the art are intended to be within the scope of the invention. Some embodiments are within the scope of the following numbered paragraphs.

1. A method for analyzing open chromatin, the method comprising:

(a) fragmenting and tagging accessible genomic DNA of the open chromatin, and

(b) labeling molecules proximal to the accessible genomic DNA.

2. The method of paragraph 1, wherein the fragmenting, tagging, and labeling is carried out by treating the open chromatin with a fusion protein comprising (a) a first enzyme that fragments and tags the accessible genomic DNA of the open chromatin, and (b) a second enzyme that labels molecules proximal to the accessible genomic DNA.

3. The method of paragraph 1 or 2, wherein the molecules proximal to the accessible genomic DNA are proteins, peptides, or RNA molecules.

4. The method of paragraph 2 or 3, further comprising the step of characterizing one or both of (a) genomic DNA fragments tagged by the first enzyme, and (b) proteins or peptides labeled with the second enzyme.

5. The method of any one of paragraphs 2 to 4, wherein the first enzyme is selected from the group consisting of a transposase, a retroviral integrase, a DNA-binding enzyme, or a variant thereof.

6. The method of paragraph 5, wherein the transposase is selected from the group consisting of a Tn transposase, a hAT transposase, a DD[E/D] transposase, and variants thereof.

7. The method of paragraph 6, wherein the Tn transposase is selected from the group consisting of Tn3, Tn5, Tn7, Tn10, Tn552, Tn903, Tn/O, TnA, and variants thereof.

8. The method of paragraph 7, wherein the Tn transposase is Tn5 or a variant thereof, such as Tn5-059.

9. The method of paragraph 5, wherein the DNA-binding enzyme is selected from the group consisting of a DNase, an MNase, a restriction enzyme, and variants thereof.

10. The method of any one of paragraphs 2 to 9, wherein the second enzyme is selected from the group consisting of a peroxidase, a biotin ligase, a catalase-peroxidase, and an oxidase.

11. The method of paragraph 10, wherein the peroxidase is selected from the group consisting of ascorbate peroxidase (APX), horseradish peroxidase (HRP), soybean ascorbate peroxidase, pea ascorbate peroxidase, Arabidopsis ascorbate peroxidase, maize ascorbate peroxidase, cytochrome c peroxidase, laccase, tyrosinase, and variants thereof.

12. The method of paragraph 11, wherein the second enzyme comprises an ascorbate peroxidase selected from APEX2, APEX, and variants thereof.

13. The method of any one of paragraphs 2 to 12, wherein the first enzyme comprises Tn5, or a variant thereof, and the second enzyme comprises APEX2, or a variant thereof.

14. The method of any one of paragraphs 2 to 13, wherein the fusion protein comprises a linker between the first and second enzymes.

15. The method of any one of paragraphs 2 to 14, wherein the fusion protein comprises a tag.

16. The method of any one of paragraphs 2 to 15, wherein the first enzyme tags genomic DNA fragments generated by the first enzyme with sequencing adaptors, and/or the second enzyme labels molecules proximal to the accessible genomic DNA with biotin.

17. The method of any one of paragraphs 2 to 16, wherein the method comprises the use of two fusion proteins, wherein the first fusion protein comprises the first enzyme fused to a portion of the second enzyme, and the second fusion protein comprises the first enzyme fused to a second portion of the second enzyme.

18. The method of paragraph 17, wherein the first and second fusion proteins are used together or are used sequentially.

19. The method of any one of paragraphs 4 to 18, wherein the characterization of the tagged genomic DNA fragments comprises sequencing.

20. The method of any one of paragraphs 4 to 19, wherein the characterization of the labeled proteins or peptides comprises mass spectrometry analysis.

21. The method of any one of paragraphs 4 to 20, further comprising cross-linking of RNA molecules proximal to accessible genomic DNA to proximal peptides and proteins, and analyzing the cross-linked RNA molecules by RNAseq.

22. The method of any one of paragraphs 1 to 21, wherein the open chromatin is obtained from cells of a subject or from cultured cells.

23. The method of paragraph 22, wherein the cells of a subject are comprised within a tissue biopsy or a blood sample.

24. The method of paragraph 23, wherein the tissue biopsy is a tumor biopsy.

25. The method of any one of paragraphs 4 to 24, comprising the step of characterizing (a) genomic DNA fragments tagged by the first enzyme, and (b) proteins or peptides labeled with the second enzyme.

26. The method of any one of paragraphs 1 to 25, further comprising the preparation of an epigenetic map of a region of the genome of a cell based on the characterization of tagged genomic DNA fragments, labeled RNA, labeled proteins, or labeled peptides.

27. A method for preparing an epigenetic profile associated with a disease or condition, the method comprising carrying out the method of any one of paragraphs 1 to 26 on a sample comprising cells of a subject having the disease or condition, or a model thereof.

28. A method for determining whether a subject has a disease or condition associated with an epigenetic profile, the method comprising carrying out a method of any one of paragraphs 1 to 27 on a sample from the subject.

29. A method for monitoring the progress of treatment a disease or condition associated with an epigenetic profile, the method comprising carrying out a method of any one of paragraphs 1 to 27 a sample from the subject (i) before and (ii) during or after treatment of the disease or condition.

30. A method for determining the effects of exposure of a subject to a biological or chemical stimulus, the method comprising carrying out a method of any one of paragraphs 1 to 27 on a sample from the subject after exposure to the biological or chemical stimulus.

31. A method for identifying the components of a cis-regulatory transcription factor network, the method comprising carrying out the method of any one of paragraphs 1 to 27 on a sample comprising cells of interest.

32. A method for identifying a target for drug development against a disease, the method comprising carrying out the method of any one of paragraphs 1 to 27 on a sample comprising cells characteristic of the disease and identifying one or more molecules, the presence or abundance of which is changed in the cells characteristic of the disease, relative to a control.

33. A fusion protein comprising (a) a first enzyme that fragments and tags accessible genomic

DNA of open chromatin, and (b) a second enzyme that labels molecules proximal to the accessible genomic DNA, or a portion thereof.

34. The fusion protein of paragraph 33, wherein the first enzyme comprises a transposase, a retroviral integrase, a DNA-binding enzyme, or a variant thereof.

35. The fusion protein of paragraph 34, wherein the transposase is selected from the group consisting of Tn transposases, hAT transposases, DD[E/D] transposases, and variants thereof.

36. The fusion protein of paragraph 35, wherein the Tn transposase is selected from the group consisting of Tn3, Tn5, Tn7, Tn10, Tn552, Tn903, Tn/O, and TnA, and variants thereof.

37. The fusion protein of paragraph 36, wherein the Tn transposase is Tn5 or a variant thereof, such as Tn5-059.

38. The fusion protein of paragraph 34, wherein the DNA-binding enzyme is selected from DNase, MNase, restriction enzymes, and variants thereof.

39. The fusion protein of paragraph 37, wherein the Tn transposase comprises the sequence of SEQ ID NO: 2, or a variant thereof.

40. The fusion protein of any one of paragraphs 33 to 39, wherein the second enzyme is selected from the group consisting of a peroxidase, a biotin ligase, a catalase-peroxidase, and an oxidase, or a portion thereof.

41. The fusion protein of paragraph 40, wherein the peroxidase is selected from the group consisting of ascorbate peroxidase (APX), horseradish peroxidase (HRP), soybean ascorbate peroxidase, pea ascorbate peroxidase, Arabidopsis ascorbate peroxidase, maize ascorbate peroxidase, cytochrome c peroxidase, laccase, tyrosinase, and variants thereof.

42. The fusion protein of paragraph 41, wherein the second enzyme comprises an ascorbate peroxidase selected from APEX2, APEX, and variants thereof.

43. The fusion protein of paragraph 42, wherein the APEX2 comprises the sequence of SEQ ID NO 4, or a variant thereof.

44. The fusion protein of any one of paragraphs 33 to 37 and 39 to 43, wherein the first enzyme comprises Tn5, or a variant thereof, and the second enzyme comprises APEX2, or a variant thereof.

45. The fusion protein of any one of paragraphs 33 to 44, wherein the first enzyme is N-terminal to the second enzyme.

46. The fusion protein of any one of paragraphs 33 to 44, wherein the second enzyme is N-terminal to the first enzyme.

47. The fusion protein of any one of paragraphs 33 to 46, comprising a linker between the first enzyme and the second enzyme.

48. The fusion protein of paragraph 47, wherein the linker comprises a sequence selected from SEQ ID NOs: 7, 9, 11, and 13.

49. The fusion protein of any one of paragraphs 33 to 48, further comprising a tag.

50. The fusion protein of paragraph 49, wherein the tag comprises a Flag tag.

51. The fusion protein of paragraph 50, wherein the Flag tag comprises the sequence of SEQ ID NO: 15 or 16.

52. A nucleic acid molecule encoding a fusion protein of any one of paragraphs 33 to 51.

53. The nucleic acid molecule of paragraph 52, comprising the sequence of SEQ ID NO: 1 or SEQ ID NO: 3.

54. A cell comprising a nucleic acid molecule of paragraph 52 or 53 or expressing a fusion protein of any one of paragraphs 33 to 51.

55. A vector comprising a nucleic acid molecule of paragraph 52 or 53.

56. A kit comprising (a) a fusion protein of any one of paragraphs 33 to 51, a nucleic acid molecule of paragraph 52 or 53, a cell of paragraph 54, or a vector of paragraph 55, and (b) one or more reagents for carrying out the method of any one of paragraphs 1 to 32.

57. A kit comprising (i) (a) a first fusion protein comprising a first enzyme that fragments and tags accessible genomic DNA of open chromatin, and (b) a first portion of a second enzyme, and (ii) a second fusion protein comprising said first enzyme and a second portion of said second enzyme, wherein said first and second portions of said second enzyme together label molecules proximal to the accessible genomic DNA.

58. A method for characterizing changes in open chromatin, the method comprising carrying out a method according to any one of paragraphs 1-26 with chromatin from or present in cells subject to different conditions or at different times, and classifying transcription factors identified as being associated with the open chromatin with respect to abundance or activity under the different conditions or at the different times.

59. The method of paragraph 58, wherein the abundance of identified transcription factors is characterized as being decreased, unchanged, or increased.

60. The method of paragraph 58 or 59, wherein the activity of identified transcription factors is characterized as being closed, unchanged, or open.

61. The method of any one of paragraphs 58 to 60, wherein both abundance and activity of identified transcription factors is classified.

62. The method of any one of paragraphs 58 to 61, wherein the different conditions are selected from exposure to drug treatment or a physiological change.

63. The method of any one of paragraphs 58 to 62, wherein the different times are different stages of development or different times before, during, or after therapeutic intervention.

64. The method of any one of paragraphs 58 to 63, further comprising determining relationships between transcription factors, determining their functions, identifying them as therapeutic targets, identifying them as transcriptional activators, or identifying them as transcriptional repressors.

65. The method of any one of paragraphs 58 to 64, further comprising the identification of transcription factor networks, and optionally associated cis-acting sequences.

66. The method of any one of paragraphs 58 to 65, further comprising identification of protein complex dynamics.

Other embodiments are within the scope of the following claims.

What is claimed is:

1. A method for analyzing open chromatin, the method comprising:
(a) fragmenting and tagging accessible genomic DNA of the open chromatin, and
(b) labeling molecules proximal to the accessible genomic DNA.

2. The method of claim 1, wherein the fragmenting, tagging, and labeling is carried out by treating the open chromatin with a fusion protein comprising (a) a first enzyme that fragments and tags the accessible genomic DNA of the open chromatin, and (b) a second enzyme that labels molecules proximal to the accessible genomic DNA.

3. The method of claim 1, wherein the molecules proximal to the accessible genomic DNA are proteins, peptides, or RNA molecules.

4. The method of claim 2, further comprising the step of characterizing one or both of (a) genomic DNA fragments tagged by the first enzyme, and (b) proteins or peptides labeled with the second enzyme.

5. The method of claim 2, wherein the first enzyme is selected from the group consisting of a transposase, a retroviral integrase, a DNA-binding enzyme, or a variant thereof.

6. The method of claim 5, wherein the transposase is selected from the group consisting of a Tn transposase, a hAT transposase, a DD[E/D] transposase, and variants thereof.

7. The method of claim 6, wherein the Tn transposase is selected from the group consisting of Tn3, Tn5, Tn7, Tn10, Tn552, Tn903, Tn/O, TnA, and variants thereof.

8. The method of claim 7, wherein the Tn transposase is Tn5 or a variant thereof, such as Tn5-059.

9. The method of claim 5, wherein the DNA-binding enzyme is selected from the group consisting of a DNase, an MNase, a restriction enzyme, and variants thereof.

10. The method of claim 2, wherein the second enzyme is selected from the group consisting of a peroxidase, a biotin ligase, a catalase-peroxidase, and an oxidase.

11. The method of claim 10, wherein the peroxidase is selected from the group consisting of ascorbate peroxidase (APX), horseradish peroxidase (HRP), soybean ascorbate peroxidase, pea ascorbate peroxidase, Arabidopsis ascorbate peroxidase, maize ascorbate peroxidase, cytochrome c peroxidase, laccase, tyrosinase, and variants thereof.

12. The method of claim 11, wherein the second enzyme comprises an ascorbate peroxidase selected from APEX2, APEX, and variants thereof.

13. The method of claim 2, wherein the first enzyme comprises Tn5, or a variant thereof, and the second enzyme comprises APEX2, or a variant thereof.

14. The method of claim 2, wherein the fusion protein comprises a linker between the first and second enzymes.

15. The method of claim 2, wherein the fusion protein comprises a tag.

16. The method of claim 2, wherein the first enzyme tags genomic DNA fragments generated by the first enzyme with sequencing adaptors, and/or the second enzyme labels molecules proximal to the accessible genomic DNA with biotin.

17. The method of claim 2, wherein the method comprises the use of two fusion proteins, wherein the first fusion protein comprises the first enzyme fused to a portion of the second enzyme, and the second fusion protein comprises the first enzyme fused to a second portion of the second enzyme.

18. The method of claim 17, wherein the first and second fusion proteins are used together or are used sequentially.

19. The method of claim 4, wherein the characterization of the tagged genomic DNA fragments comprises sequencing.

20. The method of claim 4, wherein the characterization of the labeled proteins or peptides comprises mass spectrometry analysis.

21. The method of claim 4, further comprising cross-linking of RNA molecules proximal to accessible genomic DNA to proximal peptides and proteins, and analyzing the cross-linked RNA molecules by RNAseq.

22. The method of claim 1, wherein the open chromatin is obtained from cells of a subject or from cultured cells.

23. The method of claim 22, wherein the cells of a subject are comprised within a tissue biopsy or a blood sample.

24. The method of claim 23, wherein the tissue biopsy is a tumor biopsy.

25. The method of claim 4, comprising the step of characterizing (a) genomic DNA fragments tagged by the first enzyme, and (b) proteins or peptides labeled with the second enzyme.

26. The method of claim 1, further comprising the preparation of an epigenetic map of a region of the genome of a cell based on the characterization of tagged genomic DNA fragments, labeled RNA, labeled proteins, or labeled peptides.

27. A method for preparing an epigenetic profile associated with a disease or condition, the method comprising carrying out the method of claim 1 on a sample comprising cells of a subject having the disease or condition, or a model thereof.

28. A method for determining whether a subject has a disease or condition associated with an epigenetic profile, the method comprising carrying out a method of claim 1 on a sample from the subject.

29. A method for monitoring the progress of treatment a disease or condition associated with an epigenetic profile, the method comprising carrying out a method of claim 1 a sample from the subject (i) before and (ii) during or after treatment of the disease or condition.

30. A method for determining the effects of exposure of a subject to a biological or chemical stimulus, the method comprising carrying out a method of claim 1 on a sample from the subject after exposure to the biological or chemical stimulus.

31. A method for identifying the components of a cis-regulatory transcription factor network, the method comprising carrying out the method of claim 1 on a sample comprising cells of interest.

32. A method for identifying a target for drug development against a disease, the method comprising carrying out the method of claim 1 on a sample comprising cells characteristic of the disease and identifying one or more molecules, the presence or abundance of which is changed in the cells characteristic of the disease, relative to a control.

33. A fusion protein comprising (a) a first enzyme that fragments and tags accessible genomic DNA of open chromatin, and (b) a second enzyme that labels molecules proximal to the accessible genomic DNA, or a portion thereof.

34. The fusion protein of claim 33, wherein the first enzyme comprises a transposase, a retroviral integrase, a DNA-binding enzyme, or a variant thereof.

35. The fusion protein of claim 34, wherein the transposase is selected from the group consisting of Tn transposases, hAT transposases, DD[E/D] transposases, and variants thereof.

36. The fusion protein of claim 35, wherein the Tn transposase is selected from the group consisting of Tn3, Tn5, Tn7, Tn10, Tn552, Tn903, Tn/O, and TnA, and variants thereof.

37. The fusion protein of claim 36, wherein the Tn transposase is Tn5 or a variant thereof, such as Tn5-059.

38. The fusion protein of claim 34, wherein the DNA-binding enzyme is selected from DNase, MNase, restriction enzymes, and variants thereof.

39. The fusion protein of claim 37, wherein the Tn transposase comprises the sequence of SEQ ID NO: 2, or a variant thereof.

40. The fusion protein of claim 33, wherein the second enzyme is selected from the group consisting of a peroxidase, a biotin ligase, a catalase-peroxidase, and an oxidase, or a portion thereof.

41. The fusion protein of claim 40, wherein the peroxidase is selected from the group consisting of ascorbate peroxidase (APX), horseradish peroxidase (HRP), soybean ascorbate peroxidase, pea ascorbate peroxidase, Arabidopsis ascorbate peroxidase, maize ascorbate peroxidase, cytochrome c peroxidase, laccase, tyrosinase, and variants thereof.

42. The fusion protein of claim 41, wherein the second enzyme comprises an ascorbate peroxidase selected from APEX2, APEX, and variants thereof.

43. The fusion protein of claim 42, wherein the APEX2 comprises the sequence of SEQ ID NO 4, or a variant thereof.

44. The fusion protein of claim 33, wherein the first enzyme comprises Tn5, or a variant thereof, and the second enzyme comprises APEX2, or a variant thereof.

45. The fusion protein of claim 33, wherein the first enzyme is N-terminal to the second enzyme.

46. The fusion protein of claim 33, wherein the second enzyme is N-terminal to the first enzyme.

47. The fusion protein of claim 33, comprising a linker between the first enzyme and the second enzyme.

48. The fusion protein of claim 47, wherein the linker comprises a sequence selected from SEQ ID NOs: 7, 9, 11, and 13.

49. The fusion protein of claim 33, further comprising a tag.

50. The fusion protein of claim 49, wherein the tag comprises a Flag tag.

51. The fusion protein of claim 50, wherein the Flag tag comprises the sequence of SEQ ID NO: 15 or 16.

52. A nucleic acid molecule encoding a fusion protein of claim 33.

53. The nucleic acid molecule of claim 52, comprising the sequence of SEQ ID NO: 1 or SEQ ID NO: 3.

54. A cell comprising a nucleic acid molecule of claim 52 or expressing a fusion protein encoded thereby.

55. A vector comprising a nucleic acid molecule of claim 52.

56. A kit comprising (a) a fusion protein of 33, a nucleic acid molecule encoding the same, a cell expressing the fusion protein, or a vector comprising said nucleic acid molecule, and (b) one or more reagents for carrying out a method of described herein.

57. A kit comprising (i) (a) a first fusion protein comprising a first enzyme that fragments and tags accessible genomic DNA of open chromatin, and (b) a first portion of a second enzyme, and (ii) a second fusion protein comprising said first enzyme and a second portion of said second enzyme, wherein said first and second portions of said second enzyme together label molecules proximal to the accessible genomic DNA.

58. A method for characterizing changes in open chromatin, the method comprising carrying out a method according to claim 1 with chromatin from or present in cells subject to different conditions or at different times, and classifying transcription factors identified as being associated with the open chromatin with respect to abundance or activity under the different conditions or at the different times.

59. The method of claim 58, wherein the abundance of identified transcription factors is characterized as being decreased, unchanged, or increased.

60. The method of claim 58, wherein the activity of identified transcription factors is characterized as being closed, unchanged, or open.

61. The method of claim 58, wherein both abundance and activity of identified transcription factors is classified.

62. The method of claim 58, wherein the different conditions are selected from exposure to drug treatment or a physiological change.

63. The method of claim 58, wherein the different times are different stages of development or different times before, during, or after therapeutic intervention.

64. The method of claim 58, further comprising determining relationships between transcription factors, determining their functions, identifying them as therapeutic targets, identifying them as transcriptional activators, or identifying them as transcriptional repressors.

65. The method of claim 58, further comprising the identification of transcription factor networks, and optionally associated cis-acting sequences.

66. The method of claim 58, further comprising identification of protein complex dynamics.


SEQUENCE LISTING (40 sequences)

SEQ ID NO: 1 - DNA, 1428 bp, Artificial Sequence
  Synthetic Construct
atgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatc

SEQ ID NO: 2 - protein, 476 aa, Artificial Sequence
  Synthetic Construct
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 3 - DNA, 747 bp, Artificial Sequence
  Synthetic Construct
ggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctattttgagctacgccgatttctaccagttggctggcgttgttgccgttgaggtcacgggtggacctaaggttccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctcgttgacaaatatgcagcggacgaagatgccttctttgctgattacgctgaggctcaccaaaagctttccgagcttgggtttgctgatgcc

SEQ ID NO: 4 - protein, 249 aa, Artificial Sequence
  Synthetic Construct
GKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADA

SEQ ID NO: 5 - protein, 249 aa, Artificial Sequence
  Synthetic Construct
GKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDATKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADA

SEQ ID NO: 6 - DNA, 15 bp, Artificial Sequence
  Synthetic Construct
ccagctccagctcca

SEQ ID NO: 7 - protein, 5 aa, Artificial Sequence
  Synthetic Construct
PAPAP

SEQ ID NO: 8 - DNA, 36 bp, Artificial Sequence
  Synthetic Construct
gctgaggctgctgctaaggaggctgctgctaaggcg

SEQ ID NO: 9 - protein, 12 aa, Artificial Sequence
  Synthetic Construct
AEAAAKEAAAKA

SEQ ID NO: 10 - DNA, 60 bp, Artificial Sequence
  Synthetic Construct
ggcggaggtggttctggcggtggaggttcaggcggtggtggaagtggcggaggtggttca

SEQ ID NO: 11 - protein, 20 aa, Artificial Sequence
  Synthetic Construct
GGGGSGGGGSGGGGSGGGGS

SEQ ID NO: 12 - DNA, 18 bp, Artificial Sequence
  Synthetic Construct
ggatccggtgcaggcgcc

SEQ ID NO: 13 - protein, 6 aa, Artificial Sequence
  Synthetic Construct
GSGAGA

SEQ ID NO: 14 - DNA, 24 bp, Artificial Sequence
  Synthetic Construct
gattacaaggatgacgacgataag

SEQ ID NO: 15 - protein, 8 aa, Artificial Sequence
  Synthetic Construct
DYKDDDDK

SEQ ID NO: 16 - protein, 22 aa, Artificial Sequence
  Synthetic Construct
DYKDHDGDYKDHDIDYKDDDDK

SEQ ID NO: 17 - protein, 9 aa, Artificial Sequence
  Synthetic Construct
YPYDVPDYA

SEQ ID NO: 18 - DNA, 1452 bp, Artificial Sequence
  Synthetic Construct
atgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatcgattacaaggatgacgacgataag

SEQ ID NO: 19 - protein, 484 aa, Artificial Sequence
  Synthetic Construct
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKIDYKDDDDK

SEQ ID NO: 20 - DNA, 774 bp, Artificial Sequence
  Synthetic Construct
atgggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctattttgagctacgccgatttctaccagttggctggcgttgttgccgttgaggtcacgggtggacctaaggttccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctcgttgacaaatatgcagcggacgaagatgccttctttgctgattacgctgaggctcaccaaaagctttccgagcttgggtttgctgatgccgattacaaggatgacgacgataag

SEQ ID NO: 21 - protein, 258 aa, Artificial Sequence
  Synthetic Construct
MGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADADYKDDDDK

SEQ ID NO: 22 - DNA, 2199 bp, Artificial Sequence
  Synthetic Construct
atgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatcggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctattttgagctacgccgatttctaccagttggctggcgttgttgccgttgaggtcacgggtggacctaaggttccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctcgttgacaaatatgcagcggacgaagatgccttctttgctgattacgctgaggctcaccaaaagctttccgagcttgggtttgctgatgccgattacaaggatgacgacgataag

SEQ ID NO: 23 - protein, 733 aa, Artificial Sequence
  Synthetic Construct
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKIGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADADYKDDDDK

SEQ ID NO: 24 - DNA, 2214 bp, Artificial Sequence
  Synthetic Construct
atgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatcccagctccagctccaggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctattttgagctacgccgatttctaccagttggctggcgttgttgccgttgaggtcacgggtggacctaaggttccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctcgttgacaaatatgcagcggacgaagatgccttctttgctgattacgctgaggctcaccaaaagctttccgagcttgggtttgctgatgccgattacaaggatgacgacgataag

SEQ ID NO: 25 - protein, 738 aa, Artificial Sequence
  Synthetic Construct
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKIPAPAPGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADADYKDDDDK

SEQ ID NO: 26 - DNA, 2235 bp, Artificial Sequence
  Synthetic Construct
atgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatcgctgaggctgctgctaaggaggctgctgctaaggcgggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctattttgagctacgccgatttctaccagttggctggcgttgttgccgttgaggtcacgggtggacctaaggttccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctcgttgacaaatatgcagcggacgaagatgccttctttgctgattacgctgaggctcaccaaaagctttccgagcttgggtttgctgatgccgattacaaggatgacgacgataag

SEQ ID NO: 27 - protein, 745 aa, Artificial Sequence
  Synthetic Construct
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKIAEAAAKEAAAKAGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADADYKDDDDK

SEQ ID NO: 28 - DNA, 2259 bp, Artificial Sequence
  Synthetic Construct
atgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatcggcggaggtggttctggcggtggaggttcaggcggtggtggaagtggcggaggtggttcaggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctattttgagctacgccgatttctaccagttggctggcgttgttgccgttgaggtcacgggtggacctaaggttccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctcgttgacaaatatgcagcggacgaagatgccttctttgctgattacgctgaggctcaccaaaagctttccgagcttgggtttgctgatgccgattacaaggatgacgacgataag

SEQ ID NO: 29 - protein, 753 aa, Artificial Sequence
  Synthetic Construct
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKIGGGGSGGGGSGGGGSGGGGSGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADADYKDDDDK

SEQ ID NO: 30 - DNA, 2217 bp, Artificial Sequence
  Synthetic Construct
atgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatcggatccggtgcaggcgccggaaagtcttacccaactgtgagtgctgattaccaggacgccgttgagaaggcgaagaagaagctcagaggcttcatcgctgagaagagatgcgctcctctaatgctccgtttggcattccactctgctggaacctttgacaagggcacgaagaccggtggacccttcggaaccatcaagcaccctgccgaactggctcacagcgctaacaacggtcttgacatcgctgttaggcttttggagccactcaaggcggagttccctattttgagctacgccgatttctaccagttggctggcgttgttgccgttgaggtcacgggtggacctaaggttccattccaccctggaagagaggacaagcctgagccaccaccagagggtcgcttgcccgatcccactaagggttctgaccatttgagagatgtgtttggcaaagctatggggcttactgaccaagatatcgttgctctatctgggggtcacactattggagctgcacacaaggagcgttctggatttgagggtccctggacctctaatcctcttattttcgacaactcatacttcacggagttgttgagtggtgagaaggaaggtctccttcagctaccttctgacaaggctcttttgtctgaccctgtattccgccctctcgttgacaaatatgcagcggacgaagatgccttctttgctgattacgctgaggctcaccaaaagctttccgagcttgggtttgctgatgccgattacaaggatgacgacgataag

SEQ ID NO: 31 - protein, 739 aa, Artificial Sequence
  Synthetic Construct
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKIGSGAGAGKSYPTVSADYQDAVEKAKKKLRGFIAEKRCAPLMLRLAFHSAGTFDKGTKTGGPFGTIKHPAELAHSANNGLDIAVRLLEPLKAEFPILSYADFYQLAGVVAVEVTGGPKVPFHPGREDKPEPPPEGRLPDPTKGSDHLRDVFGKAMGLTDQDIVALSGGHTIGAAHKERSGFEGPWTSNPLIFDNSYFTELLSGEKEGLLQLPSDKALLSDPVFRPLVDKYAADEDAFFADYAEAHQKLSELGFADADYKDDDDK

SEQ ID NO: 32 - protein, 5 aa, Artificial Sequence
  Synthetic Construct
EAAAK

SEQ ID NO: 33 - protein, 12 aa, Artificial Sequence
  Synthetic Construct
AEAAAKEAAAKA

SEQ ID NO: 34 - protein, 5 aa, Artificial Sequence
  Synthetic Construct
GGGGS

SEQ ID NO: 35 - DNA, 19 bp, Artificial Sequence
  Synthetic Construct
ctgtctcttatacacatct

SEQ ID NO: 36 - DNA, 33 bp, Artificial Sequence
  Synthetic Construct
tcgtcggcagcgtcagatgtgtataagagacag

SEQ ID NO: 37 - DNA, 34 bp, Artificial Sequence
  Synthetic Construct
gtctcgtgggctcggagatgtgtataagagacag

SEQ ID NO: 38 - DNA, 50 bp, Artificial Sequence
  Synthetic Construct
aatgatacggcgaccaccgagatctacactcgtcggcagcgtcagatgtg

SEQ ID NO: 39 - DNA, 53 bp, Artificial Sequence
  Synthetic Construct
caagcagaagacggcatacgagattcgccttagtctcgtgggctcggagatgt

SEQ ID NO: 40 - DNA, 34 bp, Artificial Sequence
  Synthetic Construct
gtgactggagttcagacgtgtgctcttccgatct